SEQUENCE LISTING (157 sequences)

SEQ ID NO: 1 - DNA, 1415 bp, Arabidopsis thaliana
catgaattcattttcccacgtccctccgggttttagatttcacccgacagatgaagaacttgtagactactacctgaggaaaaaagtcgcatcgaagagaatagaaattgatttcataaaggacattgatctttacaagattgagccatgggaccttcaagagttgtgcaaaattgggcatgaagagcagagtgattggtacttctttagccataaagacaagaagtatcccacagggactcgaaccaatagagcaacaaaagcagggttttggaaagccaccggaagagataaggctatctatttgaggcatagtctaattggcatgaggaaaacacttgtgttttacaagggaagagccccaaatggacaaaagtctgattggatcatgcacgaataccgcttagaaaccgatgaaaacggaactcctcaggaagaaggatgggttgtgtgtagggttttcaagaagagattggctgcagttagacgaatgggagattacgactcatccccttcacattggtacgatgatcaactttcttttatggcctccgagctcgagacaaacggtcaacgacggattctccccaatcatcatcagcagcagcagcacgagcaccaacaacatatgccatatggcctcaatgcatctgcttacgctctcaacaaccctaacttgcaatgcaagcaagagctagaactacactacaaccacctggtacaacgaaatcatcttcttgatgaatctcatttatcgttcctccaacttcctcaactagaaagccctaagattcaacaagataacagtaattgcaactctcttccttatggaacaagcaacatcgataataactcgagccataatgctaacttgcagcaatcaaatatcgcgcatgaggaacaattgaatcaaggaaatcagaacttcagctctctatacatgaacagcggcaacgagcaagtgatggaccaagtcacagactggagagttctcgataaatttgttgcttctcagctaagcaacgaggaggctgccacagcttctgcatctatacagaataatgccaaggacacaagcaatgctgagtaccaagttgatgaagaaaaagatccgaaaagggcttcagacatgggagaagaatatactgcttctacttcttcgagttgtcagattgatctatggaagtgagctgaaagagaagacatataaatgcatatatacatatatatatatacgtacacacgaacactaatcaagtgtagatgatgatgatggtacagatttatatttgctttgattgattcttactacattattgaacttatgtcatatgcatatatacattgcgtatctatgcatatttatacttgtactcaatatgattaaccatatataaactctaatctaaatgtaa

SEQ ID NO: 2 - protein, 395 aa, Arabidopsis thaliana
MNSFSHVPPGFRFHPTDEELVDYYLRKKVASKRIEIDFIKDIDLYKIEPWDLQELCKIGHEEQSDWYFFSHKDKKYPTGTRTNRATKAGFWKATGRDKAIYLRHSLIGMRKTLVFYKGRAPNGQKSDWIMHEYRLETDENGTPQEEGWVVCRVFKKRLAAVRRMGDYDSSPSHWYDDQLSFMASELETNGQRRILPNHHQQQQHEHQQHMPYGLNASAYALNNPNLQCKQELELHYNHLVQRNHLLDESHLSFLQLPQLESPKIQQDNSNCNSLPYGTSNIDNNSSHNANLQQSNIAHEEQLNQGNQNFSSLYMNSGNEQVMDQVTDWRVLDKFVASQLSNEEAATASASIQNNAKDTSNAEYQVDEEKDPKRASDMGEEYTASTSSSCQIDLWK

SEQ ID NO: 3 - DNA, 1077 bp, Arabidopsis thaliana
atggctgataataaggtcaatctttcgattaatggacaatcaaaagtgcctccaggtttcagattccatcccaccgaagaagaacttctccattactatctccgtaagaaagttaactctcaaaagatcgatcttgatgtcattcgtgaagttgatctaaacaagcttgagccttgggatattcaagaggaatgtagaatcggttcaacgccacaaaacgactggtacttcttcagccacaaggacaagaagtatccaaccgggaccaggacgaaccgggcaacagtcgctggattctggaaagctaccggacgtgacaaaatcatctgcagttgtgtccggagaattggactgaggaagacactcgtgttctacaaaggaagagctcctcacggtcagaaatccgactggatcatgcatgagtatcgcctcgacgatactccaatgtctaatggctatgctgatgttgttacagaagatccaatgagctataacgaagaaggttgggtggtatgtcgagtgttcaggaagaagaactatcaaaagattgacgattgtcctaaaatcactctatcttctttacctgatgacacggaggaagagaaggggcccacctttcacaacactcaaaacgttaccggtttagaccatgttcttctctacatggaccgtaccggttctaacatttgcatgcccgagagccaaacaacgactcaacatcaagatgatgtcttattcatgcaactcccaagtcttgagacacctaaatccgagagcccggtcgaccaaagtttcctgactccaagcaaactcgatttctctcccgttcaagagaagataaccgaaagaccggtttgcagcaactgggctagtcttgaccggctcgtagcttggcaattgaacaatggtcatcataatccgtgtcatcgtaagagttttgatgaagaagaagaaaatggtgatactatgatgcagcgatgggatcttcattggaataatgatgataatgttgatctttggagtagtttcactgagtcttcttcgtctttagacccacttcttcatttatctgtatga

SEQ ID NO: 4 - protein, 358 aa, Arabidopsis thaliana
MADNKVNLSINGQSKVPPGFRFHPTEEELLHYYLRKKVNSQKIDLDVIREVDLNKLEPWDIQEECRIGSTPQNDWYFFSHKDKKYPTGTRTNRATVAGFWKATGRDKIICSCVRRIGLRKTLVFYKGRAPHGQKSDWIMHEYRLDDTPMSNGYADVVTEDPMSYNEEGWVVCRVFRKKNYQKIDDCPKITLSSLPDDTEEEKGPTFHNTQNVTGLDHVLLYMDRTGSNICMPESQTTTQHQDDVLFMQLPSLETPKSESPVDQSFLTPSKLDFSPVQEKITERPVCSNWASLDRLVAWQLNNGHHNPCHRKSFDEEEENGDTMMQRWDLHWNNDDNVDLWSSFTESSSSLDPLLHLSV

SEQ ID NO: 5 - DNA, 924 bp, Arabidopsis thaliana
atgagttcgtctaacggaggtgtaccgcctggttttcggtttcatccaacggacgaagaacttcttcattactacttgaagaagaagatttcttatgaaaagtttgagatggaagtcatcaaagaggttgatttgaacaagattgagccgtgggatttacaagatagatgcaaaataggatcaacgccacaaaatgagtggtatttcttcagccacaaggataggaaatatccgacagggtcaaggacaaaccgtgcaacccactccggtttttggaaggcgacaggacgtgacaaatgcataagaaattcttacaagaagatcggcatgaggaagactcttgtattctacaaaggaagagctcctcatggccaaaagacagattggatcatgcacgagtaccgtatcgaagacactgaagatgacccttgtgaagatggatgggttgtttgtagagtgttcaagaagaaaaatctgttcaaagtagggaatgatgttggctcaaacatcagcaataataggcttgaagctcgtagtttcatccgaagagaaagcccttaccaaggaatctcaatgtttgagcttaacaagcctgaagagattagtgttcatcaatatcctcaaccaccaatgtttcaacctcatcacaagcctctttcaataggctatgactattcattggctcttctaccaagagaaagcgagtaccaacaagcgtgtcagccatcaggggttgaggttggcacgtgcaaggcagtaagtgaatggggaatagtgaattgtaacatggtgagtcatgaggactcgtctagagcgatgaggtttgaagatgacggtaacaacacttcttccacggtccagccaccttctaatctgctttcattgcgcggtgaaaatggatttcttgggttgttttaaacagat

SEQ ID NO: 6 - protein, 305 aa, Arabidopsis thaliana
MSSSNGGVPPGFRFHPTDEELLHYYLKKKISYEKFEMEVIKEVDLNKIEPWDLQDRCKIGSTPQNEWYFFSHKDRKYPTGSRTNRATHSGFWKATGRDKCIRNSYKKIGMRKTLVFYKGRAPHGQKTDWIMHEYRIEDTEDDPCEDGWVVCRVFKKKNLFKVGNDVGSNISNNRLEARSFIRRESPYQGISMFELNKPEEISVHQYPQPPMFQPHHKPLSIGYDYSLALLPRESEYQQACQPSGVEVGTCKAVSEWGIVNCNMVSHEDSSRAMRFEDDGNNTSSTVQPPSNLLSLRGENGFLGLF

SEQ ID NO: 7 - DNA, 1185 bp, Arabidopsis thaliana
atgaattcgttttcacaagtacctcctggcttcagatttcatcctactgatgaagaacttgtagactactacttgaggaaaaaagttgcatcaaagagaatagaaatcgatatcatcaaggatgttgatctttacaagattgagccatgtgatcttcaagagttatgcaagataggaaacgaagagcagagcgaatggtacttctttagtcataaagacaagaagtatcccacgggaactcgaaccaatagagccacgaaagcaggattttggaaagccactggaagagacaaggctatatatataagacatagtcttatcggtatgaggaaaacacttgtgttttacaaaggaagagccccaaatggtcagaaatccgattggatcatgcacgaatatcgcttagaaacaagtgaaaatggaacccctcaggaagaaggatgggtagtatgtagggtattcaagaagaaattggcagcgacagtgaggaaaatgggagattaccattcatcaccatcgcagcattggtacgatgatcagctctcttttatggcctccgagatcatttctagttctccacgacagtttcttcccaatcatcattataaccgccaccatcaccagcagacattgccttgtggcctcaatgcattcaacaacaacaatcctaacttgcaatgcaagcaagagctcgagttacattacaatcaaatggtacaacatcaacaacaaaaccatcatcttcgtgaatctatgtttctccagcttcctcagctcgaaagccctaccagtaattgcaattctgacaacaacaataacacaagaaatattagtaacttgcagaaatcatcaaatatatctcatgaggaacaattgcaacaagggaatcaaagtttcagctctctgtattacgatcaaggagtagagcaaatgactactgactggagagttctcgataaatttgttgcttcacagcttagcaatgatgaagaggctgcagccgtggtttcttcttcttctcatcaaaacaacgtcaagattgacacgagaaacacgggttatcatgtgatagatgagggaataaatttgccggagaatgattctgaaagggttgttgaaatgggagaagagtattcaaatgctcatgctgcttctacttcttcaagttgtcagattgatctctag

SEQ ID NO: 8 - protein, 394 aa, Arabidopsis thaliana
MNSFSQVPPGFRFHPTDEELVDYYLRKKVASKRIEIDIIKDVDLYKIEPCDLQELCKIGNEEQSEWYFFSHKDKKYPTGTRTNRATKAGFWKATGRDKAIYIRHSLIGMRKTLVFYKGRAPNGQKSDWIMHEYRLETSENGTPQEEGWVVCRVFKKKLAATVRKMGDYHSSPSQHWYDDQLSFMASEIISSSPRQFLPNHHYNRHHHQQTLPCGLNAFNNNNPNLQCKQELELHYNQMVQHQQQNHHLRESMFLQLPQLESPTSNCNSDNNNNTRNISNLQKSSNISHEEQLQQGNQSFSSLYYDQGVEQMTTDWRVLDKFVASQLSNDEEAAAVVSSSSHQNNVKIDTRNTGYHVIDEGINLPENDSERVVEMGEEYSNAHAASTSSSCQIDL

SEQ ID NO: 9 - DNA, 1168 bp, Arabidopsis thaliana
atcttgtttcttactcatttctctaacaattttccaaattaaatacgtttataggatcatcgtggatggataatataatgcaatcgtcaatgccaccgggattccgatttcatccgacagaggaagagcttgtgggttattacctagataggaagatcaattcaatgaagagtgctttagatgtcattgtagagattgatctctacaaaatggagccatgggatatacaagcgaggtgtaaactagggtatgaagagcaaaacgagtggtacttctttagtcataaggacaggaagtaccctaccgggactaggaccaaccgagccactgcggctgggttctggaaagccacgggtagagacaaggcggtactatcaaaaaacagtgtcatcggaatgcggaagacacttgtctactacaagggtcgagctcctaatggaagaaagtccgattggatcatgcacgaataccgtctccaaaactccgagcttgccccggttcaggaggaaggctgggtggtgtgtcgagcatttaggaagccaattccaaaccagaggccattagggtacgagccatggcagaaccagctctaccacgtcgaaagtagtaacaactactcatcttcagtgacaatgaacacgagtcatcatatcggtgcatcttcatcaagtcataaccttaatcaaatgctcatgagcaataaccactacaatcctaataatacatcctcatcgatgcatcaatatggcaacattgagctcccgcagttggacagcccgagcttgtcgcctagtttagggacgaataaagatcagaacgagagtttcgagcaagaagaagagaagagctttaactgtgtggattggagaacactagataccttgcttgagacacaagtcatacatccgcataaccctaatattcttatgttcgaaacgcagtcgtataatccggcgccaagcttcccttccatgcatcaaagctataatgaggtcgaagctaatattcatcattctcttggatgcttccctgactcgtaattaaaaaaaaacacacttctatatattgatttgtattctatgatttaattgttccatctggaaaataacatttataactgtctatttgtaaaagaagtttgtgtttattacgtagaaatttgttgttg

SEQ ID NO: 10 - protein, 324 aa, Arabidopsis thaliana
MDNIMQSSMPPGFRFHPTEEELVGYYLDRKINSMKSALDVIVEIDLYKMEPWDIQARCKLGYEEQNEWYFFSHKDRKYPTGTRTNRATAAGFWKATGRDKAVLSKNSVIGMRKTLVYYKGRAPNGRKSDWIMHEYRLQNSELAPVQEEGWVVCRAFRKPIPNQRPLGYEPWQNQLYHVESSNNYSSSVTMNTSHHIGASSSSHNLNQMLMSNNHYNPNNTSSSMHQYGNIELPQLDSPSLSPSLGTNKDQNESFEQEEEKSFNCVDWRTLDTLLETQVIHPHNPNILMFETQSYNPAPSFPSMHQSYNEVEANIHHSLGCFPDS

SEQ ID NO: 11 - DNA, 1534 bp, Arabidopsis thaliana
cactccctcacacttcctcatatatatcatgtgtacttttcgtcttaaacatctctctattttcatttcttttctttgattacttaagaacatgactaagtctctatcattaaacctctcttgtttaagaagcagacaaagatcgttcagtttgcagcaccttcatgtttctcttggacattcaggttttacttatgtgggtatatgtaatagaagattcatcttttgttttaaaattaaagggcaaaggtcgaaaagaaagcgtcaaagtttgcttaaagaagagtaaggataatggagatagggtcgtcatccacggtggctggcggaggacagctttcggtgcctccgggattccggtttcatccaacggaggaggagcttctttactattacctcaagaagaaggtttcttatgaaccaattgatttagatgtaattagagaagttgatctcaacaagctggaaccttgggaacttaaagagaaatgcagaatcgggtcgggtcctcaaaacgagtggtatttctttagccacaaggacaagaaatatccgactgggacccgaacgaaccgggcgacagcagccgggttctggaaagctactggtagagacaaatccatacatctcaacagctccaagaagattggacttcgtaagactcttgtcttctacactggtcgtgctcctcatggccaaaaaaccgaatggatcatgcacgaatatcgcttggacgatagcgaaaacgaaattcaggaagatgggtgggttgtatgcagagtgttcaagaagaagaatcatttcagaggatttcaccaagaacaagagcaagatcatcatcatcatcaccaatacataagcaccaacaatgatcatgatcaccatcatcacattgactccaattcaaacaatcattctcctttgatcctacatcctctggatcatcatcaccaccatcatcacattggacgacaaatccacatgccattacatgagtttgcaaacactctgagccatggctcaatgcatctacctcagctctttagccctgactcagctgcagcggcggctgccgcagcagcatcagcccaaccatttgtgtcgccaatcaacactacggacattgaatgttcacagaatctactgaggttgacttctaacaacaactatggtggagactggtcgtttctggacaagcttctcactacaggcaacatgaatcagcaacagcagcagcaagtgcagaaccatcaagcaaaatgttttggtgacttgagtaacaacgataataacgatcaggctgatcatttgggtaacaacaatggtggttcttcttcatctccagttaatcaaaggtttccgtttcactacttgggaaacgacgctaatcttctcaagttcccaaagtagatcaatcggatttgttgaatctcatgtttttcgattttattctctctcttttttagctgtgcgaacttgacacagcaaccaactcacagttcgaggaatctgtttgtttttttgttttttataa

SEQ ID NO: 12 - protein, 371 aa, Arabidopsis thaliana
MEIGSSSTVAGGGQLSVPPGFRFHPTEEELLYYYLKKKVSYEPIDLDVIREVDLNKLEPWELKEKCRIGSGPQNEWYFFSHKDKKYPTGTRTNRATAAGFWKATGRDKSIHLNSSKKIGLRKTLVFYTGRAPHGQKTEWIMHEYRLDDSENEIQEDGWVVCRVFKKKNHFRGFHQEQEQDHHHHHQYISTNNDHDHHHHIDSNSNNHSPLILHPLDHHHHHHHIGRQIHMPLHEFANTLSHGSMHLPQLFSPDSAAAAAAAAASAQPFVSPINTTDIECSQNLLRLTSNNNYGGDWSFLDKLLTTGNMNQQQQQQVQNHQAKCFGDLSNNDNNDQADHLGNNNGGSSSSPVNQRFPFHYLGNDANLLKFPK

SEQ ID NO: 13 - DNA, 1098 bp, Arabidopsis thaliana
atggagccaatggaatcttgtagcgttcctccaggatttaggttccatccgacggacgaagagcttgtcgggtactatctaaggaagaaaatcgcatcgcaaaagattgatctcgacgtcatcagagacatcgatctctacagaatagaaccatgggatctacaagaacaatgtcgaatcggttatgaggaacaaaatgaatggtatttttttagtcacaaggacaagaaatatccaacggggacaagaactaatagagcgaccatggctggattttggaaagccacgggaagagacaaagctgtttacgacaaaacaaaactaattggtatgaggaaaacacttgtgttctacaaaggacgtgcacctaatggcaagaaatccgattggatcatgcatgagtaccggctcgagtcagatgagaatgcaccgccccaggaagaaggatgggtggtttgtagagcattcaaaaaaagagctacagggcaagccaagaacacggaaacttggagctcaagttacttttacgatgaagttgcaccgaatggagttaactcggttatggaccccattgattacatatctaagcagcaacataacatttttgggaaaggtttgatgtgtaagcaagaactagaaggaatggttgatggtataaactatatacaatcgaatcaattcattcagctcccacaactccaaagcccttctctcccgctgatgaaaagaccttcaagctcgatgtccataacatcaatggataacaattacaactataaactcccattagcggatgaagaaagcttcgagtcattcataagaggagaggatagaaggaagaagaaaaagcaagtaatgatgacgggaaattggagagagttagacaagtttgttgcttcacaacttatgagccaagaagacaatggaacttcaagtttcgcaggtcatcatatagttaatgaagataaaaacaacaatgatgtggagatggattcgtcaatgtttttgagcgaaagagaagaagaaaacaggttcgtcagtgaattcttgagtacaaactcggattatgatattgggatttgcgtatttgataattga

SEQ ID NO: 14 - protein, 365 aa, Arabidopsis thaliana
MEPMESCSVPPGFRFHPTDEELVGYYLRKKIASQKIDLDVIRDIDLYRIEPWDLQEQCRIGYEEQNEWYFFSHKDKKYPTGTRTNRATMAGFWKATGRDKAVYDKTKLIGMRKTLVFYKGRAPNGKKSDWIMHEYRLESDENAPPQEEGWVVCRAFKKRATGQAKNTETWSSSYFYDEVAPNGVNSVMDPIDYISKQQHNIFGKGLMCKQELEGMVDGINYIQSNQFIQLPQLQSPSLPLMKRPSSSMSITSMDNNYNYKLPLADEESFESFIRGEDRRKKKKQVMMTGNWRELDKFVASQLMSQEDNGTSSFAGHHIVNEDKNNNDVEMDSSMFLSEREEENRFVSEFLSTNSDYDIGICVFDN

SEQ ID NO: 15 - DNA, 1382 bp, Arabidopsis thaliana
atctccaactttcagtatgatatatagttacaattaaataaacctcacatgctctattcttgcttgatttttgagttaatcttgaatctctttgatgatgtcaaaatctatgagcatatcagtgaacggacaatctcaagtgcctcctgggtttaggtttcatccgaccgaggaagagctgttgcagtattatctccggaagaaagttaatagcatcgagatcgatcttgatgtcattcgcgacgttgatctcaacaagctcgagccttgggacattcaagagatgtgtaaaataggaacaacgccacaaaacgactggtatttctttagccacaaggacaaaaaatatccgacgggaacgagaactaacagagccactgcggctggattttggaaagcaactggccgcgacaagatcatatatagcaatggccgtagaattgggatgagaaagactcttgttttctacaaaggccgagctcctcacggccaaaaatctgattggatcatgcatgaatatagactcgatgacaacattatttcccccgaggatgtcaccgttcatgaggtcgtgagtattataggggaagcatcacaagacgaaggatgggtggtgtgtcgtattttcaagaagaagaatcttcacaaaaccctaaacagtcccgtcggaggagcttccctgagcggcggcggagatacgccgaagacgacatcatctcagatcttcaacgaggatactctcgaccaatttcttgaacttatggggagatcttgtaaagaagagctaaatcttgaccctttcatgaaactcccaaacctcgaaagccctaacagtcaggcaatcaacaactgccacgtaagctctcccgacactaatcataatatccacgtcagcaacgtggtcgacactagctttgttactagctgggcggctttagaccgcctcgtggcctcgcagcttaacggacccacatcatattcaattacagccgtcaatgagagccacgtgggccatgatcatctcgctttgccttccgtccgatctccgtaccccagcctaaaccggtccgcttcgtaccacgccggtttaacacaggaatatacaccggagatggagctatggaatacgacgacgtcgtctctatcgtcatcgcctggcccattttgtcacgtgtcgaatggtagtggataacggaaatggagggaaagagagagaggcacacacacataagacatttcacatttgtgtaaaaagatataatcataccatatagttgttgaaacatataaaagaaagtcagatttgtgataattacagtggttatgtgaaatattgtaaaatagattaatataagaatttaaaagaggagaataaagatctc

SEQ ID NO: 16 - protein, 365 aa, Arabidopsis thaliana
MMSKSMSISVNGQSQVPPGFRFHPTEEELLQYYLRKKVNSIEIDLDVIRDVDLNKLEPWDIQEMCKIGTTPQNDWYFFSHKDKKYPTGTRTNRATAAGFWKATGRDKIIYSNGRRIGMRKTLVFYKGRAPHGQKSDWIMHEYRLDDNIISPEDVTVHEVVSIIGEASQDEGWVVCRIFKKKNLHKTLNSPVGGASLSGGGDTPKTTSSQIFNEDTLDQFLELMGRSCKEELNLDPFMKLPNLESPNSQAINNCHVSSPDTNHNIHVSNVVDTSFVTSWAALDRLVASQLNGPTSYSITAVNESHVGHDHLALPSVRSPYPSLNRSASYHAGLTQEYTPEMELWNTTTSSLSSSPGPFCHVSNGSG

SEQ ID NO: 17 - DNA, 1227 bp, Arabidopsis thaliana
cgttctattttcttctcctttttctatctttagcaatgaacatatcagtaaacggacagtcacaagtacctcctggctttaggtttcacccaaccgaggaagagctcttgaagtattacctccgcaagaaaatctctaacatcaagatcgatctcgatgttattcctgacattgatctcaacaagctcgagccttgggatattcaagagatgtgtaagattggaacgacgccgcaaaacgattggtacttttatagccataaggacaagaagtatcccaccgggactagaaccaacagagccaccacggtcggattttggaaagcgacgggacgtgacaagaccatatataccaatggtgatagaatcgggatgcgaaagacgcttgtcttctacaaaggtcgagcccctcatggtcagaaatccgattggatcatgcacgaatatagactcgacgagagtgtattaatctcctcgtgtggcgatcatgacgtcaacgtagaaacgtgtgatgtcataggaagtgacgaaggatgggtggtgtgtcgtgttttcaagaaaaataacctttgcaaaaacatgattagtagtagcccggcgagttcggtgaaaacgccgtcgttcaatgaggagactatcgagcaacttctcgaagttatggggcaatcttgtaaaggagagatagttttagaccctttcttaaaactccctaacctcgaatgccataacaacaccaccatcacgagttatcagtggttaatcgacgaccaagtcaacaactgccacgtcagcaaagttatggatcccagcttcatcactagctgggccgctttggatcggctcgttgcctcacagttaaatgggcccaactcgtattcaataccagccgttaatgagacttcacaatcaccgtatcatggactgaaccggtccggttgtaataccggtttaacaccagattactatataccggagattgatttatggaacgaggcagatttcgcgagaacgacatgccacttgttgaacggtagtggataacggaaatggaagagaggaagtgactaagtgagtgacataccaaaacattgtgtagaaagatataataatcatcataccatatatatagttgttgaaacttttacggtataaatggtcagattgtgataattacagtacgtagttatgtgatatcattacagaaaataaaagttctctatatctgtgt

SEQ ID NO: 18 - protein, 334 aa, Arabidopsis thaliana
MNISVNGQSQVPPGFRFHPTEEELLKYYLRKKISNIKIDLDVIPDIDLNKLEPWDIQEMCKIGTTPQNDWYFYSHKDKKYPTGTRTNRATTVGFWKATGRDKTIYTNGDRIGMRKTLVFYKGRAPHGQKSDWIMHEYRLDESVLISSCGDHDVNVETCDVIGSDEGWVVCRVFKKNNLCKNMISSSPASSVKTPSFNEETIEQLLEVMGQSCKGEIVLDPFLKLPNLECHNNTTITSYQWLIDDQVNNCHVSKVMDPSFITSWAALDRLVASQLNGPNSYSIPAVNETSQSPYHGLNRSGCNTGLTPDYYIPEIDLWNEADFARTTCHLLNGSG

SEQ ID NO: 19 - DNA, 1057 bp, Arabidopsis thaliana
atgggttcttcgtcgaacggaggagtgccacctggtttccggtttcatccgacggacgaagagcttctccattactacttgaagaagaaaatctcttaccaaaagtttgagatggaagtcatcagagaggttgacttaaacaagcttgagccttgggatttgcaagagagatgcaagataggatcaacaccacaaaacgaatggtacttcttcagccacaaggacaggaaatatccgacggggtcaaggaccaaccgtgctactcatgcagggttctggaaggcgacgggacgtgacaagtgcataaggaactcttacaaaaagataggaatgaggaagacacttgtgttctacaaaggtagagctcctcatggccaaaagactgattggatcatgcatgagtaccgtcttgaagacgctgatgatcctcaagccaaccctagtgaagatggatgggtggtatgtagagtgtttatgaagaaaaatttgttcaaggtagtaaatgaaggtagctcaagcattaactcattggaccaacacaaccatgacgcatctaacaacaaccatgcacttcaagctcgtagctttatgcaccgagacagtccataccagctagtacgtaaccacggagccatgacattcgaacttaacaagcctgaccttgctcttcatcaatacccaccaatcttccacaagccaccttcacttggatttgactactcttcaggacttgcaagggacagtgagagtgcggctagtgaagggttacaataccagcaagcgtgtgagccgggtttagacgttggtacatgtgagacagtggctagtcataatcatcaacaaggtctaggtgaatgggcaatgatggatagacttgtgacttgtcacatgggaaatgaagattcctctagagggattacgtatgaggatggtaacaacaattcgtcctctgtggttcagccagttcccgcgacgaaccagctaacattgcgtagtgagatggatttctggggttattctaaatagatgtttatatgtctttcttatgagtctacga

SEQ ID NO: 20 - protein, 341 aa, Arabidopsis thaliana
MGSSSNGGVPPGFRFHPTDEELLHYYLKKKISYQKFEMEVIREVDLNKLEPWDLQERCKIGSTPQNEWYFFSHKDRKYPTGSRTNRATHAGFWKATGRDKCIRNSYKKIGMRKTLVFYKGRAPHGQKTDWIMHEYRLEDADDPQANPSEDGWVVCRVFMKKNLFKVVNEGSSSINSLDQHNHDASNNNHALQARSFMHRDSPYQLVRNHGAMTFELNKPDLALHQYPPIFHKPPSLGFDYSSGLARDSESAASEGLQYQQACEPGLDVGTCETVASHNHQQGLGEWAMMDRLVTCHMGNEDSSRGITYEDGNNNSSSVVQPVPATNQLTLRSEMDFWGYSK

SEQ ID NO: 21 - DNA, 1465 bp, Arabidopsis thaliana
ctttctcttcacattcaccgaaacttataaccaagatcaatatcagttctctctctcgatgaacatcatcttctacacagccatctttctttgacttcttcttcttcttcttaatataacggctcgtttcttttgtttccaaacgagtaaatagtgtcctatacacatatctataattccacaggttgaagaaaagaaataatggaatcggtggatcaatcatgtagtgttcctccgggattcagattccatccaacagatgaagagctcgttggttactatttaaggaaaaaagttgcatcgcaaaagatcgatcttgatgtcataagagatattgatctctacagaatcgaaccatgggatttacaagagagctgccgaatcggatatgaggaaagaaatgaatggtatttcttcagccacaaagataagaaatatccaacaggaacaagaacaaacagagcgaccatggctgggttttggaaagccacgggccgagacaaggctgtttacgacaagtcaaaactgattggtatgagaaaaacacttgtgttctacaaaggaagagcccctaatggccaaaaaaccgattggatcatgcatgaataccggctagagtcagatgagaatgcacctcctcaggaagaagggtgggtggtttgtagagcgttcaagaaaaagccaatgaccgggcaagccaagaacacagaaacttggagctcaagttacttttacgacgaattaccgagtggagtacgctcagttacggagcctcttaactacgtatctaagcagaaacaaaacgtttttgcacaagatttaatgttcaagcaagaactagaaggatcagatatcggtttaaacttcatccactgcgatcaattcattcaacttccgcagcttgaaagcccttcactccctcttaccaaaagaccagtgagcttgacgtcaattacatcattggagaagaataaaaatatctacaaaagacatttaatagaagaggatgtgagcttcaatgcgctaataagtagtggaaataaagataagaagaagaagaaaacatcagtgatgacgacggattggagagcactcgataaatttgttgcttctcaacttatgagccaagaagatggagtttcgggttttggaggtcatcatgaagaagataacaataaaatcggtcattacaataacgaagagagcaataacaagggatcagtagagactgcttcttccacgttattgagtgatagagaagaagagaacagattcatcagtggattattgtgttccaacttggactatgacttatatagggatttacatgtttgataaaagataacactataatatggtgcgtaacgtttgctatataggtacgtagaatttattgatacagtataacatatataaagatgtgtgaaagatttgttttcttctactatatatactttttcgtgaa

SEQ ID NO: 22 - protein, 377 aa, Arabidopsis thaliana
MESVDQSCSVPPGFRFHPTDEELVGYYLRKKVASQKIDLDVIRDIDLYRIEPWDLQESCRIGYEERNEWYFFSHKDKKYPTGTRTNRATMAGFWKATGRDKAVYDKSKLIGMRKTLVFYKGRAPNGQKTDWIMHEYRLESDENAPPQEEGWVVCRAFKKKPMTGQAKNTETWSSSYFYDELPSGVRSVTEPLNYVSKQKQNVFAQDLMFKQELEGSDIGLNFIHCDQFIQLPQLESPSLPLTKRPVSLTSITSLEKNKNIYKRHLIEEDVSFNALISSGNKDKKKKKTSVMTTDWRALDKFVASQLMSQEDGVSGFGGHHEEDNNKIGHYNNEESNNKGSVETASSTLLSDREEENRFISGLLCSNLDYDLYRDLHV

SEQ ID NO: 23 - DNA, 1047 bp, Arabidopsis thaliana
atggaaagtctcgcacacattcctcccggttatcgattccatccgaccgatgaagaactcgttgactattatctcaagaacaaagttgcattcccgggaatgcaagttgatgttatcaaagatgttgatctctacaaaatcgagccatgggacatccaagagttatgtggaagagggacaggagaagagagggaatggtatttctttagccacaaggacaagaaatatccaactgggacacgaaccaatagagcaacgggctccggattttggaaagcaacgggtcgagacaaggccatttactcaaagcaagagcttgttgggatgaggaagactcttgtcttttacaaaggtagggccccaaatggtcagaaatctgattggataatgcacgaataccgtcttgagaccgatgaaaatggaccgcctcatgaggaaggatgggtggtttgtcgcgctttcaagaagaagctaaccacgatgaactacaacaatccaagaacaatgatgggatcatcatcaggccaagaatctaactggttcacgcagcaaatggatgtggggaatggtaattactatcatcttcctgatctagagagtccgagaatgtttcaaggctcatcatcatcatcactatcatcattacatcagaatgatcaagacccttatggtgtcgtactcagcactattaacgcaaccccaactacaataatgcaacgagatgatggtcatgtgattaccaatgatgatgatcatatgatcatgatgaacacaagtactggtgatcatcatcaatcaggattactagtcaatgatgatcataatgatcaagtaatggattggcaaacgcttgacaagtttgttgcttctcagctaatcatgagccaagaagaggaagaagttaacaaagatccatcagataattcttcgaatgaaacatttcatcatctctctgaagagcaagctgcaacaatggtttcgatgaatgcttcttcctcttcttctccatgttccttctactcttgggctcaaaatacacacacgtaa

SEQ ID NO: 24 - protein, 348 aa, Arabidopsis thaliana
MESLAHIPPGYRFHPTDEELVDYYLKNKVAFPGMQVDVIKDVDLYKIEPWDIQELCGRGTGEEREWYFFSHKDKKYPTGTRTNRATGSGFWKATGRDKAIYSKQELVGMRKTLVFYKGRAPNGQKSDWIMHEYRLETDENGPPHEEGWVVCRAFKKKLTTMNYNNPRTMMGSSSGQESNWFTQQMDVGNGNYYHLPDLESPRMFQGSSSSSLSSLHQNDQDPYGVVLSTINATPTTIMQRDDGHVITNDDDHMIMMNTSTGDHHQSGLLVNDDHNDQVMDWQTLDKFVASQLIMSQEEEEVNKDPSDNSSNETFHHLSEEQAATMVSMNASSSSSPCSFYSWAQNTHT

SEQ ID NO: 25 - DNA, 1178 bp, Arabidopsis thaliana
tcggttgatcgggtttcttctatgatcgatctttctaagatcaagaagaaaccgcagcgaaataaatcctagaaaataactatcaatatgatgaaggttgatcaagattattcgtgtagtataccgcctggatttaggtttcatccgacagatgaagaacttgtcggatattatctcaagaagaaaatcgcctcccagaggattgatctcgacgttatcagagaaattgatctttacaagatcgaaccatgggatctacaagagagatgtaggatagggtacgaggagcaaacggagtggtatttcttcagccatagagacaagaagtatccgactgggactaggacaaaccgagccaccgtggccggtttctggaaagcaacgggccgggacaaggcggtttacctcaactccaaacttatcggtatgagaaaaacgcttgtcttttaccgaggtcgagcgcctaatggccaaaagtccgattggatcattcacgaatactacagcctcgagtcacaccagaactctcctccacaggaagaaggatgggtagtgtgtagagcatttaagaaacgaacgaccatcccaacaaaaaggaggcaactttgggatccgaactgcttattctacgacgacgccactctcttggaacctctcgacaagcgagccagacataatcctgattttaccgccacaccgttcaagcaagaactactctccgaggccagtcacgtccaggatggagatttcggatctatgtaccttcaatgcatcgatgatgatcaattctcccagcttcctcagctcgagagcccctctcttccgtcggaaataactccccatagtactactttttctgagaacagtagccggaaagatgacatgagctccgagaagaggatcactgactggagatatctagataagttcgtggcgtctcaatttttgatgagtggagaagactaaaaaaggctttcctatgcatgcatgcactagaaacgtcgtcgcattttggatttacatatatggtcgtaataattcaccgaaggaattgatttcgatggataaatagaaggtgagaatcgaataagaagatatatatagggctttacacttactcagtttgtttcgtataatgtattagtgactcgaaaatatacgttcaaattacggtgatt

SEQ ID NO: 26 - protein, 292 aa, Arabidopsis thaliana
MMKVDQDYSCSIPPGFRFHPTDEELVGYYLKKKIASQRIDLDVIREIDLYKIEPWDLQERCRIGYEEQTEWYFFSHRDKKYPTGTRTNRATVAGFWKATGRDKAVYLNSKLIGMRKTLVFYRGRAPNGQKSDWIIHEYYSLESHQNSPPQEEGWVVCRAFKKRTTIPTKRRQLWDPNCLFYDDATLLEPLDKRARHNPDFTATPFKQELLSEASHVQDGDFGSMYLQCIDDDQFSQLPQLESPSLPSEITPHSTTFSENSSRKDDMSSEKRITDWRYLDKFVASQFLMSGED

SEQ ID NO: 27 - DNA, 1322 bp, Hordeum vulgare
gaattcggcacgaggcctcgtgccgaattcggcacgaggatcacaaccatccagccagccagccagccgcctttctcttctctttcaacgccgggcgcccgcggagggccggtggagcgagcgagcgagcgaggagggggaggatgatgacggcaatggtgaaggccgaggcgatgacggcggaggcggaggggagcagcgggcggagggacgcggaggcggagctcaacctgccgccgggcttccgcttccaccccacggacgacgagctcgtcgtgcactacctctgcaggaaggtggcgggacagccgcagcccgtgccaatcatcgccgaggtcgatctctacaagttcaacccctgggatctgcccgagagggcgctgttcgggagcagggagtggtacttcttcacgccgcgcgaccgcaagtaccccaacggctcgcgccccaaccgctccgccggcaccggctactggaaggccacgggcgccgacaagcccgtggcgcccagggagagcggcggcaggaccgtcggcatcaagaaggcgctcgtcttctactccggcagggcgcccaggggcgtcaagaccgactggatcatgcacgagtaccgcatcgcccaagccgaccgcacacccggcaagaagggatccctcaagctggacgaatgggtgctgtgccggctgtataacaagaagaacaactgggacaaggtcaaggtggagcaggacatggctgtggtgcagggacagaatggggaggtcatggacgcgctggccaccgacgccatgtccgacagcttccagacgcacgactcctcggagatcgacaacgcctccggcctgcagcagcagcaccacggcttcatggacatggcgcagcggcaggccagggaaggcatggtgacggtcaaggaggacagcgactggttcaccggcctgagtatggacgacctgcagacttgttacatgaacatggggcagatggtgaatccagcggcgatgcctgtgcacgacggcagcggctatctgcagtcgatgaactcgcctcagatgatgagacccatgtggcaaacaatcttgccaccattctgagatggaagaagaaagatatggtgtaaattatgtttgaaatagaagtgattagacaaatacttactttgaactagaaagagatagaaatttcccaggcatgattctgaagatgtggtggtaggcttgtagcagtatttattctttggtttcgatctataaatttggtattctaaacaaacatgtaaattttattcccagataatctcaagtctaaacaccaaattttag

SEQ ID NO: 28 - protein, 316 aa, Hordeum vulgare
MMTAMVKAEAMTAEAEGSSGRRDAEAELNLPPGFRFHPTDDELVVHYLCRKVAGQPQPVPIIAEVDLYKFNPWDLPERALFGSREWYFFTPRDRKYPNGSRPNRSAGTGYWKATGADKPVAPRESGGRTVGIKKALVFYSGRAPRGVKTDWIMHEYRIAQADRTPGKKGSLKLDEWVLCRLYNKKNNWDKVKVEQDMAVVQGQNGEVMDALATDAMSDSFQTHDSSEIDNASGLQQQHHGFMDMAQRQAREGMVTVKEDSDWFTGLSMDDLQTCYMNMGQMVNPAAMPVHDGSGYLQSMNSPQMMRPMWQTILPPF

SEQ ID NO: 29 - DNA, 1225 bp, Hordeum vulgare
accatcaccacacaacccaactcctctcactccccaacaatctcaaacaacagctcgaggaatcaacagcagcgactactctgttcgttgaaccaacaatcaacagacggacatggggatgccggcggcgaggaggagggagagggacgcggaggcggagctgaacctgcccccgggattccgcttccaccccaccgacgacgagctggtggagcactacctgtgccgcaaggcggcggggcagcgcctgcccgtgcccatcatcgccgaggtcgacctctaccgcttcgacccgtgggcgctccccgaccgcgccctcttcggcacccgcgagtggtacttcttcacgccccgcgaccgcaagtaccccaacggatcccgccccaaccgcgccgccggcaacggctactggaaggccaccggcgccgacaagcccgtcgcgccccgcgggggtcgcaccatggggatcaagaaggccctcgtgttctacgccggcaaggcgcccaagggggtcaagacggactggatcatgcatgagtaccgcctcgccgacgccggacgcgccgccgccagcaagaagggctccctcaggctggacgactgggtgctctgccggctgtacaacaagaagaacgagtgggagaagatgcagctgcagcagcagcagggggaaggggagaccatgatggagcccaaggcggaggagaacacggcgtccgacatggtcgtcacctcgcactcccactcccagtcccagtcgcactcgcactcgtggggcgaggcgcgcacgcccgagtcggagatcgtcgacaacgacccgtcgctgttccaccaggcgcaggcgacggcgttccagacgcagagccccgcggccgccgcggcgcaccaggagatgatggccaccctgatggtgcccaagaaggagggcggccgatgagggccagaagcaactgttcggggacctgaggtaccaccacgtccaagacatgtgcgacggccttgaaattgatgccccccggggacgaactgatttaatcctctttgtttccctccccagggtccgggggggcccagccgggtccgaggggatgccacgccggtgttgggcaccagaaagggggccctcgcaccgtcgatccagtcgaggagacgggcaggtaaattttttgcgtcggcgaaggaccgatatttgggcgctttggagtgcctgttgggacgcggaggg

SEQ ID NO: 30 - protein, 274 aa, Hordeum vulgare
MGMPAARRRERDAEAELNLPPGFRFHPTDDELVEHYLCRKAAGQRLPVPIIAEVDLYRFDPWALPDRALFGTREWYFFTPRDRKYPNGSRPNRAAGNGYWKATGADKPVAPRGGRTMGIKKALVFYAGKAPKGVKTDWIMHEYRLADAGRAAASKKGSLRLDDWVLCRLYNKKNEWEKMQLQQQQGEGETMMEPKAEENTASDMVVTSHSHSQSQSHSHSWGEARTPESEIVDNDPSLFHQAQATAFQTQSPAAAAAHQEMMATLMVPKKEGGR

SEQ ID NO: 31 - DNA, 993 bp, Hordeum vulgare
  misc_feature (928)..(928): n is a, c, g, or t
ttcggcacgagggaggatcggtagcagtgcagcgatcgcgaagatgagctctattggcatgatggaggcgaagatgccgccggggttccggttccacccacgggacgaggagctggtcctcgactacctcctccacaagctcaccggccggcgcgcatacggtggcgtcgacatcgtggacgtcgacctcaacaagtgcgagccatgggaccttccagaggcggcgtgcgtaggcggcagggagtggtacttcttcagcctgcgcgaccgcaagtacgccaccgggcagcgcaccaaccgcgccacgcgctccggttactggaaggccaccggcaaggaccgcgccatcctcgcccacggcgccggcgaggcgttggtggggatgcgcaagacgctcgtcttctaccaggggagggcccccaaggggacgaggacggagtgggtcatgcacgagttccgcctggaggaggaacgacgccaccaccagccgcagcagcagcacaagggccgcgccgccgcccccgaggcgaggtgccagctcaaggaagactgggtgctatgcagggtgttctacaagagcagaacaaccagcccaaggccaccatctgaagaagcctgcacattcttcaccgagctggaccttccgactatgccgcccctcgcgcccctcatcggcgcctacatcgccttcgacagcggcaccacgatgaacaccaccgagcaagtgtcctgcttctccggcctgccggcactaccgctcaggggatcggtgagcttcggggacctgctgggctgggacaaccctgagaagaaggccatcaggacagcactgagcaacatgtcaagtaacaacaattccaagttggagttgcctccaaactggagccaggagaacggcttgccacagatgtggacacccctctgaatnctttgatatgagattactgtgctgctagttgaagtgtggtggtgactagtaagtaattagatgac

SEQ ID NO: 32 - protein, 293 aa, Hordeum vulgare
MSSIGMMEAKMPPGFRFHPRDEELVLDYLLHKLTGRRAYGGVDIVDVDLNKCEPWDLPEAACVGGREWYFFSLRDRKYATGQRTNRATRSGYWKATGKDRAILAHGAGEALVGMRKTLVFYQGRAPKGTRTEWVMHEFRLEEERRHHQPQQQHKGRAAAPEARCQLKEDWVLCRVFYKSRTTSPRPPSEEACTFFTELDLPTMPPLAPLIGAYIAFDSGTTMNTTEQVSCFSGLPALPLRGSVSFGDLLGWDNPEKKAIRTALSNMSSNNNSKLELPPNWSQENGLPQMWTPL

SEQ ID NO: 33 - DNA, 1087 bp, Hordeum vulgare
cggcacgaggcataagtgatctcgagtgtgtacaccaccacatagccaattgcatcccatagagccaccgccatggcaggccaccttcaagttcagcagcaacaacaacaacagttggagcttccaccggggttcaggttccaccctactgatgaggagatcatcacctcctaccttgcccctaaggttctcaacccagcgttcgaagccacggcgatcggggaggtggacatgaacaagaacgagccatgggagcttcccaagaaggccaagatgggtcagaatgagtggtacttctactgccaaaaggaccgcaagtaccccaccgggatacgaaccaaccgggccaccaaggttggctactggaaggccacgggaaaggacaaggaaatcttcaacccacattgcacgtccatgctcatcggcatgaagaaaacattggtgttctacaagggcagagctcccagcggggagaagaccaactgggtcatgcacgagtacaggctcgagatcagcaagcagtcaacatccggcctgcccacctccatcgcaaatgccgctgccattaacatgtcttccaaggagtatgtggtttgcaggatcttccagaagaacactggaagcgggccgtcatccatggtgtcatatgaggacgttgggaccggtccgagcaacaacgaccagagcaaccgcggtgcaacagatggcatgttgaagtacttataatcagatgatccaagacaacatatatatatatatatatatatatatatatatatatatatatatatatatatgtgtgtgtgtgtgtgtgtgtgtgtgtgtgtgtgtgtatgcactaattaatcatccatcacaagatcgctcgatatataccaacatctatatgggtttgcacctcattttatttttcagcgtgaatgcaagagagtttgcatgctttgcctcgtttggcttgcatctcttctgctagtattgtgtgtatgtgtattggcttgtacatgcaccacacaccggtgtatgcctagctacatttgcattgtaaactacctaagagtgtataccatgcatgtttgaaacctatatg

SEQ ID NO: 34 - protein, 217 aa, Hordeum vulgare
MAGHLQVQQQQQQQLELPPGFRFHPTDEEIITSYLAPKVLNPAFEATAIGEVDMNKNEPWELPKKAKMGQNEWYFYCQKDRKYPTGIRTNRATKVGYWKATGKDKEIFNPHCTSMLIGMKKTLVFYKGRAPSGEKTNWVMHEYRLEISKQSTSGLPTSIANAAAINMSSKEYVVCRIFQKNTGSGPSSMVSYEDVGTGPSNNDQSNRGATDGMLKYL

SEQ ID NO: 35 - DNA, 3065 bp, Hordeum vulgare
  misc_feature (2694)..(2694): n is a, c, g, or t
cctccccgcgtcctccccctaaaaatccccaaatccgaccaaaccctaggcaaaatccactcccctccgcttccttcgctgctaagtatatcccctgggctccgaatctcctaccgggttgggtcttgtagtcgttggatgagagcttgggacagtggaggaggatgacggtgatggagctcaaggcgctaccgctcggtttccggttccaccccaccgacgaggagctcgtcaggcactacctcaaggggaagatcacggggcagatcaagaacgaggtcgaggtgatcccggagatcgacgtctgcaagtgcgagccgtgggacctcccagataaagctttgatccgctcggaggatccagagtggttcttctttgcacccaaggaccgcaaatatcccaatggaagcaggtcaaacagggcgacggaggccgggtactggaaggctaccgggaaggacagggtcatcaagtccaagggcgagaagaaaaagcagcatatgattggcatgaagaagacccttgtgttccaccgaggacgtgctccaaaaggggagcgcactggttggattatgcacgagtaccgcaccaccgagccagagtttgagtctggcgagcagggtggctatgttctgtaccgcctatttcaaaagcaagtggagaaaactgagcgctccattccggaggaaatggatagaagtggctactctcccactccatctcgttctactcctgacaatatggaagcaaacgaggaagctatcacactcataaataaggaatctcctgaatctggtctgcatggatgtccaattgagttgccatgtacaattgaaactctgggcgccccggttacaaggtggcttgcagaccgaaatggcaatcccgggacagatgaagcaaacgatgtaggcatgccttttcatcgtggtgtcgatgaaagtcccaaggccgatctgtcagttggctctctggctcacctaattgattcacagacgaaaaatctcggttctcatgagtttgccaccgtttctgcccccatgttaccggatgaccatttggacaatcttctacttcatttaactcataattcggtggaagatttcttgaatgaaacaattgctgatccagacgagctttcgtcaactgcatgtaaagttcagtacgattcagacaatgggattatgcaggctcagggtgatttgctatatgatgatccaaactggtttggtaattttctgtcagatgacacaaatccacaaccaagtggattatatgagaatgcaccattgcttccttatggcactaatcctgatgtactttccatggactccggcgacgagtccttgcaagatttgttcaatagcatggacgattcaagttggcagaatgatgtatggagcaacggatttggatttaatcccatgcatcagcagttacagtctactgtgcatccgaactatatattttctcaacagggcattgcacgcaggaggcttcggctactagattcattgtctgatgtcaatgttgagagtagagagagcatgaccagagatgaacatgaagatgaagaatcagatattgtaacttcgaaatataggagtgaatctgttgaatcatctgcagatgtagatgagtctgagtcaaccggggttactattatgagccggcgccgtgctctaactaaaagtgtgccttccgaccgcgatgatgctgaatcaacggggattactattatgggccggcgctctgctccaagttcaaatgcgccttccgatgaagctgatgctgaggcaacagggattaatattgtgagccgacgcactgctccaagttcaagcacagataactcatttaccaccgaacaggggactgcagtgcgaaggttgcggctacaatcgaaccttgacgcaggatcatgttccagtgttgatggtttgtcaagttgcattatagaacatggaagtgaaaacgaagggctggaagctgagattgacgagcatgtggatacaaacttccttgatgatgttggttttgctggcaacagtcatgctgatgagcatatgtacatgcctgatcatgaagctaattctcttacgaggctgcggaagacagcagagaaaagcgacaaggagaacaagcaggagtgtggtccccagtcacatgtgagagcaccaaggaagaggggaggcttcgcagcacgcatgatcttgccagttctgtcggtggctcttctcgttcttgttagcgttggggtctacggatgggcataatagctctcttaaaatgggatttaatgtgtcacattttccccacatcggctgtgagcattgtacatactgtgtcttactgcccgcgtttagacggttgtaagacagagaagtgtttgttcccctttcggcggggaagtgtaagtgaggtagcaaatgagaagcgggatgaagcaaggagcaagtgttgctgtagatgcctgtgacttggtacatgctagttttcgtttttctgtcaccgcaggtgttggcaaccaggttgtagtcatccattatgcgtgttgttgtgatgttgtaaggtctgaacctaattagatctgttgcagatcctttttcccattttgtggaactttgcagtcgcttcatttttagtgatgcagttgcttcatttttagtgattcatctcccaattttaagcggngtgaaaannaaaatanaanannnnnnannnnnnnnntnntccttgggggggggccccggaccccaatcccccttatagggccctnttccacaaagtgtgtattttgatagagtaatacggttgtcaagcgaggatgattgaatgggcaggagagaagagggtgtagatggaaagaagaggcgagagggagagcgaagaaaggtagtggaccgagggggtggagtaagagaagtgtaggaagaagggggtgagagtaggatagagaggagggtagggagcagttaggaagagagaggaggagagaaggagataggcaaggagagagagcagagagctggggtagagtgcgagtagatagagtcggaggagtg

SEQ ID NO: 36 - protein, 703 aa, Hordeum vulgare
MTVMELKALPLGFRFHPTDEELVRHYLKGKITGQIKNEVEVIPEIDVCKCEPWDLPDKALIRSEDPEWFFFAPKDRKYPNGSRSNRATEAGYWKATGKDRVIKSKGEKKKQHMIGMKKTLVFHRGRAPKGERTGWIMHEYRTTEPEFESGEQGGYVLYRLFQKQVEKTERSIPEEMDRSGYSPTPSRSTPDNMEANEEAITLINKESPESGLHGCPIELPCTIETLGAPVTRWLADRNGNPGTDEANDVGMPFHRGVDESPKADLSVGSLAHLIDSQTKNLGSHEFATVSAPMLPDDHLDNLLLHLTHNSVEDFLNETIADPDELSSTACKVQYDSDNGIMQAQGDLLYDDPNWFGNFLSDDTNPQPSGLYENAPLLPYGTNPDVLSMDSGDESLQDLFNSMDDSSWQNDVWSNGFGFNPMHQQLQSTVHPNYIFSQQGIARRRLRLLDSLSDVNVESRESMTRDEHEDEESDIVTSKYRSESVESSADVDESESTGVTIMSRRRALTKSVPSDRDDAESTGITIMGRRSAPSSNAPSDEADAEATGINIVSRRTAPSSSTDNSFTTEQGTAVRRLRLQSNLDAGSCSSVDGLSSCIIEHGSENEGLEAEIDEHVDTNFLDDVGFAGNSHADEHMYMPDHEANSLTRLRKTAEKSDKENKQECGPQSHVRAPRKRGGFAARMILPVLSVALLVLVSVGVYGWA

SEQ ID NO: 37 - DNA, 2303 bp, Hordeum vulgare
tttctcctggaaccctaagccccctcaggtacatgacccacccttcctcgtcctcctcctccgccccggaggccgccccggatgacccgacctccctcgccccgggcttccgcttccacccaacagacgaggagctcgtctcctactacctcaagcgcaaggtgctcggccgcccgctcaaggtcgacgccatcgccgaagtagacctctacaagctcgagccatgggacctccccgcgaggtcgcgcctccgcagccgcgactcccagtggtacttcttcagccgcctcgaccgcaagcatgccaaccgcgcgcgcaccaaccgcgccactgcaggaggctactggaagaccaccggcaaggacagggaggtccgccatggcgctagggtcgtcgggatgaagaagacgctcgtcttccacgctggccgcgcgcctaagggcgagcgcaccaactgggtcatgcacgagtatcgccttgagggcgatggcgcagccgggatcccacaggattcatttgtagtgtgccgaatcttccagaaggctgggcctggtcctcaaaacggggcacagtatggagcacccttcgtcgaggaggagtgggaggctgatgaggatgatgattttggtcctctgcctgtggagaggaacctcgttgttgagcatgaggctcctggtgctatggagaaaggatatcttcagatgaatgatctcattcaggacttggcgggtcaaaatgagaacggcagtgttgctttgccagtttctgacacttcaaataccagcagccattctgaagatgtagaaggaaattcaggggacattttgaatgacccaagtcttggttctaattttcttcagtatattcatcctggagaacaaaatagcccaatgcttaacggaaatatgttatctaatgccaatgttggagattttcttaatagttctagcccgaatgatgaattcctagagctgaaggatttggagttacctataggcaatgattccactatctggccttctgatggttgggcctggaaaactcctttctcctcggatgctgtaaatggggccaacaatgaggttcctccgatcattggtgatcagcctttccagccagatgagctggcccagttgttgcagacactacaagatgactcctccccgttgggctcaactagcactgatcttccatactcttctattacaaattctgtcaagccagaagatgattctctgatgttttttgatgcgccctttgacacgttgttcagtgatggatttagacagacgaatggatttcttggctccccagcaaccattctatctggtatcgagacactggatgatggtatcccatactatgatgcaatggacgataacttatttaatgatatgatgtgctctgtacagcagtcggctgggagcagctcccatgtctttaatggcccagttcttactcaagaggtcaacaatcccaactatacatatagtccaactcaaaaggttgtagaacctaattttgtagttggtgccccgtcgtccgctaggttacctgaagctggtagtcagttaaattgtgttgttttaccagattctcgggctaagaatgggtccatgggaaagcgttttgtaaagatgctggattcaatctcggctccccctgcttttgcagcagagttcccaggcaaatccttgtctggtgtgcaccctaacaccatcagtgtgtctactgaagtaatcagcataggaagcttaactgttgcttctcgacaaggtaagtggtcgttccagaaggatgaagacatggagctgcttttctcttccacaggtttccagcctgataatcgcatacactatggtggctgcaacacggtgactgcggtgctgcgtggcggcttctgtctcttttttctgtcagcaataatgctcctggtgagctacgaggtgggcctgtgcatctacggcaagtaacctgcccgcccgcccggtatcggtataaaaagattattatgacgttaggtaattgtatgtttattgaggcttgaactggggatctccttgcatgacatgacctccttgtgcgtttgttaaattcgacagcattagcggtaccgttttgcagccccgagagttagtgctgtatctatgaactcatccagtgctattagtattttagaactgcttaataatcagtgcatggtgttgctgcaattattcatgttcgttaggcagttttagcgtggaatgttgtaaatacaaaatagtattaagttggcggttaatcccagactcaggatgctt

SEQ ID NO: 38 - protein, 646 aa, Hordeum vulgare
MTHPSSSSSSAPEAAPDDPTSLAPGFRFHPTDEELVSYYLKRKVLGRPLKVDAIAEVDLYKLEPWDLPARSRLRSRDSQWYFFSRLDRKHANRARTNRATAGGYWKTTGKDREVRHGARVVGMKKTLVFHAGRAPKGERTNWVMHEYRLEGDGAAGIPQDSFVVCRIFQKAGPGPQNGAQYGAPFVEEEWEADEDDDFGPLPVERNLVVEHEAPGAMEKGYLQMNDLIQDLAGQNENGSVALPVSDTSNTSSHSEDVEGNSGDILNDPSLGSNFLQYIHPGEQNSPMLNGNMLSNANVGDFLNSSSPNDEFLELKDLELPIGNDSTIWPSDGWAWKTPFSSDAVNGANNEVPPIIGDQPFQPDELAQLLQTLQDDSSPLGSTSTDLPYSSITNSVKPEDDSLMFFDAPFDTLFSDGFRQTNGFLGSPATILSGIETLDDGIPYYDAMDDNLFNDMMCSVQQSAGSSSHVFNGPVLTQEVNNPNYTYSPTQKVVEPNFVVGAPSSARLPEAGSQLNCVVLPDSRAKNGSMGKRFVKMLDSISAPPAFAAEFPGKSLSGVHPNTISVSTEVISIGSLTVASRQGKWSFQKDEDMELLFSSTGFQPDNRIHYGGCNTVTAVLRGGFCLFFLSAIMLLVSYEVGLCIYGK

SEQ ID NO: 39 - DNA, 1080 bp, Oryza sativa
atgcatcctaatggcgcgccgctggccgtgccaccgggtttccggttccatccgacggacgaggaattgctctactactacctgaggaagaaggttgcttatgaggccatagatctcgacgtcatcagggagattgacctcaacaagcttgagccttgggatctcaaagatcgatgcaggattgggacgggggcgcaggaggagtggtacttcttcagccacaaggacaagaagtacccgacggggacgaggaccaaccgcgccacggtggccggcttctggaaggccaccggccgtgacaaggccatcttcctcggcagcggcggcggcacgaggattggcctgaggaagacgctggtgttctacaccggaagggccccgcacggcaagaagaccgactggatcatgcacgagtaccgcctcgacgacgacaatgtcgacgtcccggaggaaggatgggtggtgtgtagggtattcaagaagaagagcatccatcagaggggcttcgaccagccggacatggcggcggcggccgacgaggacgagctccggtaccagctgctccacggcgcgggcatgtcgtcgtcgccggtcgaccagaagcacgtgctcctccaagagcagctcgtcgcgcacggcgcccatggcggcggcttcgtcgtcccggcgttcgaagcctccatgcacctcccgcagctcgccagcgccgacgcggcgccgtgcggcggcggcggcggcggccatgtcgcgttcgcctccatgaacccgctcgacgccgccggctgcggctcgcagaacatgatgacgatgaagatggcagcgacatccggtggcgagatgctgctgatgagcggcggcggcgtcgacggcggccgcttcggcgccgccgccgactggtcgatccttgacaagctcctggcgtcgcaccagaacctcgaccagctcttccatggcaaggtcgccggagcgcatcagcagcagcagcagatggccatggacgccgcctcgtcgttgcagaggttgccgttccaccattacctcgggttggaagctgcagatcttctcaagttttctatgtag

SEQ ID NO: 40 - protein, 359 aa, Oryza sativa
MHPNGAPLAVPPGFRFHPTDEELLYYYLRKKVAYEAIDLDVIREIDLNKLEPWDLKDRCRIGTGAQEEWYFFSHKDKKYPTGTRTNRATVAGFWKATGRDKAIFLGSGGGTRIGLRKTLVFYTGRAPHGKKTDWIMHEYRLDDDNVDVPEEGWVVCRVFKKKSIHQRGFDQPDMAAAADEDELRYQLLHGAGMSSSPVDQKHVLLQEQLVAHGAHGGGFVVPAFEASMHLPQLASADAAPCGGGGGGHVAFASMNPLDAAGCGSQNMMTMKMAATSGGEMLLMSGGGVDGGRFGAAADWSILDKLLASHQNLDQLFHGKVAGAHQQQQQMAMDAASSLQRLPFHHYLGLEAADLLKFSM

SEQ ID NO: 41 - DNA, 1953 bp, Oryza sativa
atggcgcccgtgagtttgcctccaggtttcaggttccaccccaccgatgaagaactaatcatctactaccttaagcggaagatcaacggaagacagatagaactcgaaatcattccagaggttgatctttacaagtgtgagccttgggatctgcctgaaaaatcctttcttccgagtaaagatctcgaatggtacttcttcagccctagagaccgcaagtacccaaatggatcaaggacgaaccgtgcaacaaaagccggatactggaaggcaactgggaaagatcgaaaagtgaactcacagaggcgtgcagttggtatgaagaagactcttgtgtactaccgtggtcgagctccacatggttctcgcaccgattgggtcatgcacgagtaccgcctcgatgagagggaatgcgagactgacactggcttacaggatgcatatgctttatgccgggtgttcaagaagacagcgcccgggccaaagatcatagagcattatggtgtggtgcaccaccatgtcgagcaacctcaatggatgacgagcagtatcgatcgctccccaacgttggacgtgtcgtgtgatggaagaggtgatgactttgagagcagcagcttctctttcccaacagagacgccaatggactccatgcacggtgggtttggaatgcagatgagtgcacctcacgaggatggcaaatggatgcagtttctgagtgaagatgccttcaacgccaccaatccattcttgacgaacccagtttctgccaacttctcatgccttccatccaaggtggatgttgcactggaatgtgcgaggctgcagcacaggctcaccttgcctcccttggaggtggaggatttcccacaggacgtcagccttgacacgaagattggcatacttcgcagtaaccccaacgaagttgacattctccaagaattcctgtcagttgcgactgcctctcaggagctaattaatggctccaccagtagctaccctgaaatgtggttaggagctagcacaagcagtgctagctacgtcaatgaactgtcctctctagttgagatgggcggcgttggcacttctaaccaccacgaatccgcaaggctacaggtcgagatcgctgacatggaggtgttcaaggacgagaagaagcgggtggagaaccttagaggagtcaagttggtgaacaatgaccttggggagattgttgtagaaggagatgaaagcaatccaacagaagacatcatcgcgcagtaccctataaaagtcactgcagataattcaggagaagccggtcatcgcatgaccgatcccactgacgtaggcggcatcgacactgccccgatcttctcgcaatctcaacctgacgactttgctgctggtttcgacgacgtcaaccctaatgcatctttcgatctgtacgagaaggttgacgtcaaccacaggctattcgtttcaagggtcgctgcggcgaagacattcttccaccgcattgagccatcgaagaaggtcagcttccactcgaacccggcagcaaccgccgtcagtaaggcgaccgagaagttccatttccccgttacgaccaaagttagtggtagggtttccatttttagcaagttcaaggcactcataagggacaagttcttgatgatgaggccatcacattcataccaaaggttgggcagcaaagaaaccacagtgaatgagctgctgcagattgtgtcactccttttagcaccgaagcaaatcaatggctgccctactgagcaagagctggtcaagaagaaggcgaaagaagtgatgaagccgggatggggtcgtgaagggagcaacaaattgtggcttccgctctccaaagggaagggcatttccagcatgtttttgagtgggaaatggacgtttctaacctccgcgttggccatcagcactccagctgagtgcgatcactaa

SEQ ID NO: 42 - protein, 650 aa, Oryza sativa
MAPVSLPPGFRFHPTDEELIIYYLKRKINGRQIELEIIPEVDLYKCEPWDLPEKSFLPSKDLEWYFFSPRDRKYPNGSRTNRATKAGYWKATGKDRKVNSQRRAVGMKKTLVYYRGRAPHGSRTDWVMHEYRLDERECETDTGLQDAYALCRVFKKTAPGPKIIEHYGVVHHHVEQPQWMTSSIDRSPTLDVSCDGRGDDFESSSFSFPTETPMDSMHGGFGMQMSAPHEDGKWMQFLSEDAFNATNPFLTNPVSANFSCLPSKVDVALECARLQHRLTLPPLEVEDFPQDVSLDTKIGILRSNPNEVDILQEFLSVATASQELINGSTSSYPEMWLGASTSSASYVNELSSLVEMGGVGTSNHHESARLQVEIADMEVFKDEKKRVENLRGVKLVNNDLGEIVVEGDESNPTEDIIAQYPIKVTADNSGEAGHRMTDPTDVGGIDTAPIFSQSQPDDFAAGFDDVNPNASFDLYEKVDVNHRLFVSRVAAAKTFFHRIEPSKKVSFHSNPAATAVSKATEKFHFPVTTKVSGRVSIFSKFKALIRDKFLMMRPSHSYQRLGSKETTVNELLQIVSLLLAPKQINGCPTEQELVKKKAKEVMKPGWGREGSNKLWLPLSKGKGISSMFLSGKWTFLTSALAISTPAECDH

SEQ ID NO: 43 - DNA, 1101 bp, Oryza sativa
atggaatcatgcgtccctcctgggttcaggttccaccccaccgacgaggagctcgtcggctactacctccgcaagaaggtcgcctcccagaagatcgacctcgacgtcatccgcgacatcgatctctaccgcatcgaaccctgggatctccaagaacattgtgggatcgggtacgatgagcaaagcgagtggtacttcttcagctacaaggacaggaagtacccgacggggacgaggacgaacagggcgacaatggcggggttctggaaggcgacggggagggacaaggcggtgcacgacaagagcaggctcatcggcatgaggaagacactcgtcttctataagggcagggcgcctaatggccagaagaccgactggatcatgcacgagtaccgcctcgagaccgacgagaacgcgccgccacaggaagaaggatgggtggtgtgccgagcattcaagaagaggacggcgtatccggcgaggagcatggtggagacgtgggactactccttgcacgagcgcaacatcatgagcgccgcggcggcggcggcgttcgccgatccgagcgcggcgtacgcgcagatgaggcggcagcacaggagcgggcggttcaagcaggaggcggagctggacggcgccgccacagccctcctccactactccagccacctcgccgagctgccgcagctcgagagcccctccgcggctgcggcgccgctccagcccaacccgagccagctggccaccgccggcgaggacgacgactgcaagggcgataatggcggtaggagggccaagaaagcccgcgccgccggcgacaaggtggcgacgaccacggactggagagcgctcgacaagttcgtcgcgtcgcagcttagccccggggagtgtggcagcatggaggcaacggcggaagccgcggcggcggcagtcgccggtgtgagctcgccgctggaccacggcgatgacgacatggcagcattgctgtttctcaacagcgacgagagagacgaggtcgacaggtggacggggttgctcggctccggcgccggcgcgagcggcgtcgacggcgacctcggaatctgtgtgtttgacaaatga

SEQ ID NO: 44 - protein, 366 aa, Oryza sativa
MESCVPPGFRFHPTDEELVGYYLRKKVASQKIDLDVIRDIDLYRIEPWDLQEHCGIGYDEQSEWYFFSYKDRKYPTGTRTNRATMAGFWKATGRDKAVHDKSRLIGMRKTLVFYKGRAPNGQKTDWIMHEYRLETDENAPPQEEGWVVCRAFKKRTAYPARSMVETWDYSLHERNIMSAAAAAAFADPSAAYAQMRRQHRSGRFKQEAELDGAATALLHYSSHLAELPQLESPSAAAAPLQPNPSQLATAGEDDDCKGDNGGRRAKKARAAGDKVATTTDWRALDKFVASQLSPGECGSMEATAEAAAAAVAGVSSPLDHGDDDMAALLFLNSDERDEVDRWTGLLGSGAGASGVDGDLGICVFDK

SEQ ID NO: 45 - DNA, 972 bp, Oryza sativa
atggaggaggggttgccgccggggttccggttccacccgacggacgaggagctggtgacgtactacctcgcccgcaaggtctccgacttcggcttcgccacccgcgccatcgccgacgtcgacctcaacaagtgcgagccatgggacctccccagcaaggcaagcatgggggagaaggagtggtacttcttcagcatgcgcgaccgcaagtacccgacggggatccgcaccaaccgcgccacggactccggctactggaagaccaccggcaaggacaaggagatcttccacggcggcgcgctcgccggcatgaagaagacgctggtgttctaccgcgggagggcgcccaagggcgccaagaccagctgggtgatgcacgagtacaggctccagtccaagttcccctacaagcccgccaaggacgagtgggtcgtctgccgcgtcttcaagaagctccagtgccacctcgccaagccccgcccgccgcacgacgacgtcgacggagacggcgcctcgccgccggagatggtggacgcctcgtcgctcggcgagcttggcgagctcgacgtctcctccatcctcctcggcggcttcgcgccgccgtcgggcgagctctgccatggcggcggcggcggagacggtttcggcgcgcacaggcttcatgtcggcgcgtacatgagctggctgcaggcggcggcggcggccaaccagggcatgttccagtggccggcggccacgcaggcggggctggtcggaggcaccgtgttcgcggcggcgcacaaggcggcggggacgatgccgttcggcggcgggtgcagccagcagcaagcgcgggacgtgggcgtttctctggcgaacgtaggaggaggcgatgcgttgttcggcggggcgccgctggcgaaggtggacatggaatgcggcgagcaggcgccgcagctggacatggacgactccacatggagagccttctga

SEQ ID NO: 46 - protein, 323 aa, Oryza sativa
MEEGLPPGFRFHPTDEELVTYYLARKVSDFGFATRAIADVDLNKCEPWDLPSKASMGEKEWYFFSMRDRKYPTGIRTNRATDSGYWKTTGKDKEIFHGGALAGMKKTLVFYRGRAPKGAKTSWVMHEYRLQSKFPYKPAKDEWVVCRVFKKLQCHLAKPRPPHDDVDGDGASPPEMVDASSLGELGELDVSSILLGGFAPPSGELCHGGGGGDGFGAHRLHVGAYMSWLQAAAAANQGMFQWPAATQAGLVGGTVFAAAHKAAGTMPFGGGCSQQQARDVGVSLANVGGGDALFGGAPLAKVDMECGEQAPQLDMDDSTWRAF

SEQ ID NO: 47 - DNA, 660 bp, Oryza sativa
tcatgcgatcgatatagctgctccatccgtttgaaggctggctgctgctggccgagggaggcgctgcatgctgctgcaagctacgaagcagtagcaattgcaggcgcgcgcgcacaagttcatattaacttgctaccagttcgggagctatactttgatccgcggaagaagatggacgcattctcccatgtcccgccaggttttcgtttccaccctactgacgaggaactcgtggattactaccttaggaagaaggtagcactgaagaagatagacttggacgtcataaaagatattgatctgtacaaaattgagccttgggacctacaagaacaatgcaagattggaaacgaggagcagaacgagtggtacttcttcagccacaaggacaagaagtacccgacgggcacacgcaccaacagggcgaccactgccggcttttggaaggccaccgggagggacaagccgatctatgtcaagaactgccttgttgggatgaggaagacgttggttttctacaggggccgggctcccaacggacagaagtcggactggatcatgcacgagtatcgcttggagaccaacgaatacggagctccccaagtaagtttgtgtactagtgcatgcatgctaccgaggattcagacttcagacacctaa

SEQ ID NO: 48 - protein, 162 aa, Oryza sativa
MDAFSHVPPGFRFHPTDEELVDYYLRKKVALKKIDLDVIKDIDLYKIEPWDLQEQCKIGNEEQNEWYFFSHKDKKYPTGTRTNRATTAGFWKATGRDKPIYVKNCLVGMRKTLVFYRGRAPNGQKSDWIMHEYRLETNEYGAPQVSLCTSACMLPRIQTSDT

SEQ ID NO: 49 - DNA, 1095 bp, Oryza sativa
atggatggatctagtatgagcagcagcagcacgcagcagcaggcgcaggttcctcctggatttcgtttccacccgacggacgaagagctggtggattactaccttcggaagaaggtggccgcaagaaggattgatctcaatgtcatcaaggacgtcgatctctacaagattgagccatgggatctgcaagagcgttgccggatcaatggcgggtcggcggcggaggagcagaacgaatggtacttcttcagccacaaggacaagaagtacccgacggggacgaggaccaaccgtgcgacggcggccgggttctggaaggcgacggggcgagacaagcccatctacgccaccaagcagcacagcctcctcgtgggcatgaggaagacgctcgtctactaccgcggccgcgcccccaacggccacaagtccgactggatcatgcacgagtaccgcctcgagaccaccgagacggccccgccgcaggaagaagggtgggtggtatgccgggtgttcaagaagagattacccacaacaagaagagattcagaccatgacgcaccttgcggcagctggtacgttgatgaagatgcaccgggcgccttcatgtctccgatgatgatcaccagatcatcaatattgcgcccacaccaacaccacgccggcatcacgctgcaagagcaacacctccacaccacttacaagcacagagacctcactactaagattcagcagctccaagtccctgcggcaggccatcatctcctcaacaccatgccccatgacctggagagttctacttcctccttccattctcttttggtctcacctgatcaccaccaaatcaacatgcaccatgctcaggctgatcccttctttgacgacatgcatgcagtcgatcaagccactaccactgactggagagttcttgacaagtttgttgcctctcagcttagcaatgatgctacaaacaagcctgcggatcattatactgatgaaggagacattcttcaggtcagtgacaagcagcaagaggtggcagccgccgattatgcgtccacatcaacgtcaagcagccaaattgatccatggaagtga

SEQ ID NO: 50 - protein, 364 aa, Oryza sativa
MDGSSMSSSSTQQQAQVPPGFRFHPTDEELVDYYLRKKVAARRIDLNVIKDVDLYKIEPWDLQERCRINGGSAAEEQNEWYFFSHKDKKYPTGTRTNRATAAGFWKATGRDKPIYATKQHSLLVGMRKTLVYYRGRAPNGHKSDWIMHEYRLETTETAPPQEEGWVVCRVFKKRLPTTRRDSDHDAPCGSWYVDEDAPGAFMSPMMITRSSILRPHQHHAGITLQEQHLHTTYKHRDLTTKIQQLQVPAAGHHLLNTMPHDLESSTSSFHSLLVSPDHHQINMHHAQADPFFDDMHAVDQATTTDWRVLDKFVASQLSNDATNKPADHYTDEGDILQVSDKQQEVAAADYASTSTSSSQIDPWK

SEQ ID NO: 51 - DNA, 675 bp, Oryza sativa
agtagttgttgtattgatcgatcgatcgatctagagacgacgatgagcatatcggtgaacgggcagtcggtggtgccaccggggttccggttccacccaacggaggaggagctcctgacatactacctgaagaagaaggtggcctcggagcgcatcgacctcgacgtcatccgcgacgtcgacctcaacaagctcgagccatgggacatccaagagaggtgcaggattgggtcagggccgcaaaacgactggtacttcttcagccacaaggacaagaagtacccgacggggacgaggacgaaccgggcaacggcggccggcttctggaaggccaccggccgcgacaaggccatctactccagctccaaccgcatcggcatgcgcaaaaccctcgtcttctacaagggccgtgccccccacggccagaaatcagactggatcatgcacgagtaccgcctcgacgacccctcctccgcctccgcctccgtctccgtcaacctcccctcctactactcttcctcctcatcgtcatcttccccaatgcacggtgtcgccggagaccaaggagctcaggaggaaggctgggtgatctgcagggtgttcaagaagaagaacctcgtgcaccacggcggccatggagggcagcccgagcaactgctcgacggtgaccgttag

SEQ ID NO: 52 - protein, 210 aa, Oryza sativa
MSISVNGQSVVPPGFRFHPTEEELLTYYLKKKVASERIDLDVIRDVDLNKLEPWDIQERCRIGSGPQNDWYFFSHKDKKYPTGTRTNRATAAGFWKATGRDKAIYSSSNRIGMRKTLVFYKGRAPHGQKSDWIMHEYRLDDPSSASASVSVNLPSYYSSSSSSSSPMHGVAGDQGAQEEGWVICRVFKKKNLVHHGGHGGQPEQLLDGDR

SEQ ID NO: 53 - DNA, 831 bp, Oryza sativa
atgcatccgagcggcggcgcgctgtcggtgccaccggggtttcggttccacccgacggacgaggagctcctctactactacctgaggaagaaggtggcctacgaggccatagatctcgacgtcataagggagatcgacctcaacaagctcgagccctgggatctcaaagatcggtgcaggatagggacggggcctcagaacgagtggtacttcttcagccacaaggacaagaagtacccgacggggacgaggacgaaccgggcgacgacggcggggttctggaaggcgacggggagggacaaggccatcttcctcgccaacgcctgccgcatcggcatgaggaagaccctcgtcttctacgtcggcagggcgccccacggcaagaagaccgactggatcatgcacgagtaccgcctcgaccaagacaacgtcgatgtccaggaagatggttgggtggtgtgtagggtattcatgaagaagagctaccagcgaggcctcaacccggcggacatggcggcggtcgacgacgacgacctcctccaccaccaccaccaccccttcccaccggcccagctccacggcggcgccgccgaccacaagcacgacggcgccggcggccaccaccaccaccacctcatgcagccgcaccaccactacgacgacttcccttccttcgacccctccatgcagctcccccagctcatgagcgccgaccagccgccgccgccaccgccgtccctcctccccggcggcaccgcctcgtcgttgcagaggcttccactgcagtatctggggtgcgaggcagccgacctgctcaggttctccaagtag

SEQ ID NO: 54 - protein, 276 aa, Oryza sativa
MHPSGGALSVPPGFRFHPTDEELLYYYLRKKVAYEAIDLDVIREIDLNKLEPWDLKDRCRIGTGPQNEWYFFSHKDKKYPTGTRTNRATTAGFWKATGRDKAIFLANACRIGMRKTLVFYVGRAPHGKKTDWIMHEYRLDQDNVDVQEDGWVVCRVFMKKSYQRGLNPADMAAVDDDDLLHHHHHPFPPAQLHGGAADHKHDGAGGHHHHHLMQPHHHYDDFPSFDPSMQLPQLMSADQPPPPPPSLLPGGTASSLQRLPLQYLGCEAADLLRFSK

SEQ ID NO: 55 - DNA, 975 bp, Oryza sativa
atggatcggcatgaggaggaggcaggagagtctccatgtgtgccgccggggttcaggtttcacccgacggaggaggagctggtgggctactacctcgccaggaaggtggcctcccagaagatcgatctcgacatcatccaggagctcgatctctacaggatcgagccatgggatctgcaagagcgttgcaagtacggtgggcatggcggcgatgagcagacggagtggtacttcttcagctacaaggaccgcaagtaccccagcgggacgaggaccaaccgcgccacggcggcgggcttctggaaggccaccggccgcgacaagccggtgctctcgtcgccgtcgacgagggtgatcgggatgaggaagacgctggtgttctacaagggtcgcgcccccaacggccggaagaccgactggatcatccacgagtaccgcctccaatcaaacgaacacgcccctactcaggaggaaggttgggtggtttgccgcgcgtttcagaagccgatgcccaaccagcagcagcacaggctgtcctacggctgcatccccggcagctacggcgccggagcctacgccgccgtccccgacaactacagcttgctgctgcaccacgacaaccctagcttcgccgggcggccattgatgagcgccgctgcctcagctctcttcgccaacaacaacaataacagcgtcgtcgaccacagcaacattctgagttcagagtccaagcttcacttctccgacatgatgccgcctctggagagccccaccatcgtcgacggcgagggctacgtctcgcaggctagcagctgcgtcgacgtcgatcagcaagccggcatcgtcgactggaacctgctcaccagcttgctgccgccgccggcgcatcagctcttccaccacctgccttccgcttctagctccaagaacagcaacaacatttcttcgtcaggcttcatcgatgaccgagactga

SEQ ID NO: 56 - protein, 324 aa, Oryza sativa
MDRHEEEAGESPCVPPGFRFHPTEEELVGYYLARKVASQKIDLDIIQELDLYRIEPWDLQERCKYGGHGGDEQTEWYFFSYKDRKYPSGTRTNRATAAGFWKATGRDKPVLSSPSTRVIGMRKTLVFYKGRAPNGRKTDWIIHEYRLQSNEHAPTQEEGWVVCRAFQKPMPNQQQHRLSYGCIPGSYGAGAYAAVPDNYSLLLHHDNPSFAGRPLMSAAASALFANNNNNSVVDHSNILSSESKLHFSDMMPPLESPTIVDGEGYVSQASSCVDVDQQAGIVDWNLLTSLLPPPAHQLFHHLPSASSSKNSNNISSSGFIDDRD

SEQ ID NO: 57 - DNA, 795 bp, Oryza sativa
gtagcaagaaggagaagaagaagaagatcatcaatgagcatctcggtgaacgggcagtcgtgcgtgccgccggggttccggttccacccgacggaggaggagctgctcaactactacctccgcaagaaggtcgcctccgagcagatcgacctcgacgtcatccgcgacgtcgacctcaacaagctcgagccatgggacatccaagagaggtgtaagattgggtcagggccgcagaacgactggtacttcttcagccacaaggacaagaagtacccgacggggacgaggacgaacagggcgacggcggccgggttctggaaggccaccggccgcgacaaggccatctacaacgccgtccaccgcattggcatgcgcaagacgctcgtcttctacaagggccgcgccccccacggccagaagtccgactggatcatgcacgagtaccgcctcgacgaccccgccaccgacaccgccgccgccacacccacggttacttctgctgctgctgcggcggcggcgatggcggcggcggcggacggcgggcaggaggatggctgggtggtgtgcagggtgttcaagaagaagcaccaccacaaggaggccggcggcggcggcggcaagcacggcggcgacggcagcgccggcgccaaggcggcgcacgcttactcctccagcgacgacgcgctcgaccagatcctgcagtacatgggcaggtcgtgcaagcaggagcacgagctccccccgccgccgaccacctgctgctccaccaccaccaccgcacggactgggccatga

SEQ ID NO: 58 - protein, 253 aa, Oryza sativa
MSISVNGQSCVPPGFRFHPTEEELLNYYLRKKVASEQIDLDVIRDVDLNKLEPWDIQERCKIGSGPQNDWYFFSHKDKKYPTGTRTNRATAAGFWKATGRDKAIYNAVHRIGMRKTLVFYKGRAPHGQKSDWIMHEYRLDDPATDTAAATPTVTSAAAAAAAMAAAADGGQEDGWVVCRVFKKKHHHKEAGGGGGKHGGDGSAGAKAAHAYSSSDDALDQILQYMGRSCKQEHELPPPPTTCCSTTTTARTGP

SEQ ID NO: 59 - DNA, 1026 bp, Oryza sativa
atggtgatcatggagtcatgtgtgcctcctgggtttaggttccaccccaccgacgaggagctcgtcggctactacctccggaagaaggtggcctcgcagaagatcgacctcgacgtcatccgcgacgtcgacctctaccgcatcgagccatgggatctccaagagcattgcaggatagggtacgaggagcagagcgagtggtacttcttcagctacaaggacaggaagtacccgacggggacgaggacgaacagggcgacgatgacggggttctggaaggcgacggggagggacaaggcggtgcgtgagaggagcaggctcatcgggatgaggaagacgctcgtcttctacaagggcagggcacccaacggccacaagaccgactggatcgtccacgagtaccgcctcgagtccgacgagaacgccccgcctcaggaagaaggctgggtggtgtgccgcgcgttcaagaagcgaaccatgcagccgccgcggagctccatcggagcatgggaggcgagctactcctaccacgaccccgccgtcttcgtcggcggcggggaacacttcaagcaagaggccgcggccgagctggacggcgtcgccgccgccgccggagctaacgccttcctgcggtactccacccgcctggccgagctcccgcagctggagagcccgccgctgccaagccaggggagccaggcggcctcggccgtcgtcgacggcgaggaagataacgccgattctagcaggcgccctggcggcggcggcggcgccgccgccgcggtgaccacggactggagggcgttcgacaagttcgtcgcgtcccagctcagccccgaggagcagcacacctgccgggccaccgacgacgacgacatggcagcgctgctgctcctcgacggcggcgggcaggaggacgacgccgggaggtggctgggctccgccgggttgctgagcgccgtggcggccgacgcgacgacggactgcggcctcggcaccagctgcgtgcctggcgacatcaactga

SEQ ID NO: 60 - protein, 341 aa, Oryza sativa
MVIMESCVPPGFRFHPTDEELVGYYLRKKVASQKIDLDVIRDVDLYRIEPWDLQEHCRIGYEEQSEWYFFSYKDRKYPTGTRTNRATMTGFWKATGRDKAVRERSRLIGMRKTLVFYKGRAPNGHKTDWIVHEYRLESDENAPPQEEGWVVCRAFKKRTMQPPRSSIGAWEASYSYHDPAVFVGGGEHFKQEAAAELDGVAAAAGANAFLRYSTRLAELPQLESPPLPSQGSQAASAVVDGEEDNADSSRRPGGGGGAAAAVTTDWRAFDKFVASQLSPEEQHTCRATDDDDMAALLLLDGGGQEDDAGRWLGSAGLLSAVAADATTDCGLGTSCVPGDIN

SEQ ID NO: 61 - DNA, 1217 bp, Populus trichocarpa
gctcatcatccaaactgttgtgctagttgcatggtgatggctacatcttctggtggtgtgcctccagggttccggttccacccgactgatgaagaactgctccattactacttgaaaaagaaggtttcgtttcagaagtttgatatggaggtcattagagaggtggacttgaacaagatggagccttgggagttgcaagagagatgtaaaattgggtcagcgcctcaaaacgagtggtatttattctgtcacaaggacaggaaatacccgactgggtcacgaaccaatagagcaacgaatgcagggttttggaaggcaacagggagggataagtgcatcaggaacagctacaagaagattggtatgaggaaaacacttgttttctatggaggaagagctcctcatggccagaagactgactggatcatgcatgagtataggcttgaagatggcgatgatgctcaaggaaaccttggtgaagatggctgggtggtatgcagggtgttcaaaaagaagaatctattcaaggtcagtggtgaaggaggaactacaagcatgaactcatctgaccaacaactccactcgtcaagcaccaatcaatctcgaactttcatgcacagagacagccaatactccctccgtcaaaaccataaccatggaaatattcaacaaccctttgtactaagcaaggctgagctagagcttcactatcctcacatggcagcacctcatcaatactctctcttccaatcccaaaccctaatgccacctaataagtctttaggttatgaccactactcgggtctccctaaggaaccaccggtcatggttaagcagctcatgtcaggcgctagggattgcgagagtggcagtgaaagtctgaggtaccacgcttgtgagccaggactagaggtgtgcacctgtgaagcacctcagcaaatggttgcaggaagagaagatcaccagggcttgaatgaatgggctatgcttgataggattgtcacttctcatttaggaaatgaagattctgcaaaaggggtgaggtttgatgatgcaagtaacgcaccatctgcgcacgcaattaatcagctcccgttacgtggggagatgatggatttttggggctatggaaaatagtgttttgtttttagttccttccatatctctttttgaatgactactatgaacatggactgaaaccggttcggaaaaatggc

SEQ ID NO: 62 - protein, 368 aa, Populus trichocarpa
MVMATSSGGVPPGFRFHPTDEELLHYYLKKKVSFQKFDMEVIREVDLNKMEPWELQERCKIGSAPQNEWYLFCHKDRKYPTGSRTNRATNAGFWKATGRDKCIRNSYKKIGMRKTLVFYGGRAPHGQKTDWIMHEYRLEDGDDAQGNLGEDGWVVCRVFKKKNLFKVSGEGGTTSMNSSDQQLHSSSTNQSRTFMHRDSQYSLRQNHNHGNIQQPFVLSKAELELHYPHMAAPHQYSLFQSQTLMPPNKSLGYDHYSGLPKEPPVMVKQLMSGARDCESGSESLRYHACEPGLEVCTCEAPQQMVAGREDHQGLNEWAMLDRIVTSHLGNEDSAKGVRFDDASNAPSAHAINQLPLRGEMMDFWGYGK

SEQ ID NO: 63 - DNA, 1181 bp, Populus trichocarpa
tgcagtttaaatcttcagttcttggaggagatgaataccttctcgcatgtccccccgggctttagattccatccaacagatgaagaacttgttgattactaccttaggaaaaaagtcgcttcaaaaagaattgacctcgatgtcattaaggatgttgatctctataaaattgaaccatgggatcttcaagaattgtgcaaactcgggactgaagatcaaaatgaatggtacttcttcagccataaagataagaagtatcctactggaactcgcactaatagagcgacaaaagctgggttttggaaagctactggaagagacaaggctatctactctaggcatagcttggttggcatgagaaaaaccctagtgttttacaaaggacgagctccaaatggacaaaagtcagattggatcatgcatgaatatcgactagaaacaaatgaaaatggaatcccccaggcaaaaggatgggttgtctgtagggtgttcaagaagcgaatgccgacaatgagaaaagttggtgactatgactcaccatgttggtatgatgaccaggtttcattcatgccagaaattgattctccaaggcgaatatctcaaccttacgcaccataccatcatcactatcattgcaagcaagaacttgagttgcaatacaatatgcctcatgaccctttcctccagcttcctcaattagaaagcccaaaagttccacaatcagcagcaactgcgagttgtaattcagttattgcatatggttttgacaggaccaatgggaacaccttgcagtcatctacccttacacaagaagaaaaaatgcagcaatgtcatcaacaaaatttaaactcacttcacaataacaataatagtgagcaagctgtagaccaagtgactgattggcgagtccttgacaaatttgttgcttcccagttaagccatgaggatgcctccaagggaaccaataacttctccagtacagccacctttaatgaagccgagcagatgaacatgcttgccaacgaatccaaaaggtcagaaattgcacagcaatatgcctcgacgtctacgtcaagttgccaaattgatctatggaagtgaacgcatataataatgattaattactagaagatgcatactaattataggaactgcacttattatggaaaaagggcatgttc

SEQ ID NO: 64 - protein, 356 aa, Populus trichocarpa
MNTFSHVPPGFRFHPTDEELVDYYLRKKVASKRIDLDVIKDVDLYKIEPWDLQELCKLGTEDQNEWYFFSHKDKKYPTGTRTNRATKAGFWKATGRDKAIYSRHSLVGMRKTLVFYKGRAPNGQKSDWIMHEYRLETNENGIPQAKGWVVCRVFKKRMPTMRKVGDYDSPCWYDDQVSFMPEIDSPRRISQPYAPYHHHYHCKQELELQYNMPHDPFLQLPQLESPKVPQSAATASCNSVIAYGFDRTNGNTLQSSTLTQEEKMQQCHQQNLNSLHNNNNSEQAVDQVTDWRVLDKFVASQLSHEDASKGTNNFSSTATFNEAEQMNMLANESKRSEIAQQYASTSTSSCQIDLWK

SEQ ID NO: 65 - DNA, 1070 bp, Populus trichocarpa
tcaggagcttgaagaatattatcagtggagatggaatcctgtgtcccaccaggctttaggtttcatccaacagaagaagaacttgtaggatactatctcaagaggaagattcactcactgaaaatcgatctagatgttatcgttgacattgatctatataacatggagccatgggacatccaagccaagtgcaagctagggtatgatgaacagaatgagtggtatttcttcagtcacaaggataggaagtatccgaccggaacacggactaacagagccactgctgccggattttggaaggcaacaggaagggacaaggccgtgctcaccaagaacagacttattgggatgaggaagaccttggtgttctacaagggacgtgcaccaaatggaaggaaaactgactggatcatgcatgaatatcgtctccaaacatctgagcatggaccccaacaggcaaaagggtgggtggtttgccgggcattcaagaaaccaatccctaatcaaagacaaggttttgaagcttggaatcatgcttattatgtcaataatatcaaccatgctagacctccttcattctcagatacagtaactactacacatacggttcatccgaatcaaagtgcaagttttcatcagccttttagctcgagttcggacctcatttcacaccaagaatttttggacaataataatcggcttgttgaactcccacaactagatagccctagtacactttcaacaagttttgctcccaaagaaggtaacttccaccagaccaatgaagattacgatgatgaaaggagcaacaatagcagccaatatatcgactggaaaagtttggacaccttacttgcatcacaagtgaatgattccagtacttcatcatttccccttcaaaattttccttctataacgcaaaactatgacctagcagctgggcagcaagaccatgttagccatttccttgactgctttcctgacatataacaaaagctcttgcacttataatgttgcgatttatttatgtaagatgatgtattcatggtaataatagtgctatagtagga

SEQ ID NO: 66 - protein, 319 aa, Populus trichocarpa
MESCVPPGFRFHPTEEELVGYYLKRKIHSLKIDLDVIVDIDLYNMEPWDIQAKCKLGYDEQNEWYFFSHKDRKYPTGTRTNRATAAGFWKATGRDKAVLTKNRLIGMRKTLVFYKGRAPNGRKTDWIMHEYRLQTSEHGPQQAKGWVVCRAFKKPIPNQRQGFEAWNHAYYVNNINHARPPSFSDTVTTTHTVHPNQSASFHQPFSSSSDLISHQEFLDNNNRLVELPQLDSPSTLSTSFAPKEGNFHQTNEDYDDERSNNSSQYIDWKSLDTLLASQVNDSSTSSFPLQNFPSITQNYDLAAGQQDHVSHFLDCFPDI

SEQ ID NO: 67 - DNA, 1328 bp, Populus trichocarpa
cataagtaaatttttgtgaaacaattaattatggggcgttttgttttaaaaacacgcgcttattttcctgtcatagataatatatatatgagcattattattgacggtaatggtggtggtgaattttatgcagcatcatcaacagttgttatgatggagtcaatggagtcttgtgtcccacctggattcaggttccatccaaccgatgaagagcttgttggctattatctaaggaagaaagttgcatcacaaaagattgatcttgatgttattagagatattgatctttacagaattgaaccatgggatctacaagagagatgctggatcggatatgaagagcagaatgagtggtacttctttagccacaaggataagaagtatccaacagggacaaggactaatagagcaaccatggctggcttttggaaggctaccgggagagacaaggcagtgtatgacaaaacaaaactcattggcatgaggaaaacccttgtcttctacaaaggaagagccccaaatgggcagaaaactcactggatcatgcatgaataccggcttgaatcagatgagaatggtcctccacaggcaagcatatcaacttatgtcactataacaccccccccccccaacacaaatggatatgtcgccctaaaggaagaaggatgggtagtttgtcgtgcatttaagaagcgaactactggccaaaccaagagcattgaagggtgggactcaagctacttctatgaggaatcaagtggggttagctcagtggtggatcctattgattatatagcaaggcaacctcaaggctttttggctcataatttcttgtgtaagcaggagatagaaggagataagttaagtttcatgcactcagaaaattttgtacagcttcctcagttaaagagcccatctgagccattaataaagaggccaacctcgtcaatgtctttgatatcagagaataataacagtaacaatgaagaggtagagcaaaatggattgtccaacaacaacacccagaaagtaactgactggagagcccttgacaagtttgttgcttctcagttgagtcaagaagaaagatatgatggtgatggtgtttcaagctttgttgaggcagaaaataactcagatttgccatttctgttgttgcaaagtggtagagatgacgggaacaagtttaatggattcttaggttcaagttctgactgtgatattggaatatgcatatttgaaaaatgaagagaggggtttgaagaggttttttctctctggtagtacgtatgtattattaatgttttatgaaccctgatgatatttgt

SEQ ID NO: 68 - protein, 405 aa, Populus trichocarpa
MGRFVLKTRAYFPVIDNIYMSIIIDGNGGGEFYAASSTVVMMESMESCVPPGFRFHPTDEELVGYYLRKKVASQKIDLDVIRDIDLYRIEPWDLQERCWIGYEEQNEWYFFSHKDKKYPTGTRTNRATMAGFWKATGRDKAVYDKTKLIGMRKTLVFYKGRAPNGQKTHWIMHEYRLESDENGPPQASISTYVTITPPPPNTNGYVALKEEGWVVCRAFKKRTTGQTKSIEGWDSSYFYEESSGVSSVVDPIDYIARQPQGFLAHNFLCKQEIEGDKLSFMHSENFVQLPQLKSPSEPLIKRPTSSMSLISENNNSNNEEVEQNGLSNNNTQKVTDWRALDKFVASQLSQEERYDGDGVSSFVEAENNSDLPFLLLQSGRDDGNKFNGFLGSSSDCDIGICIFEK

SEQ ID NO: 69 - DNA, 1367 bp, Populus trichocarpa scaff
atcatatattttcaagcctcactacctataatgcctgaggatatgatgaatctatcaataaatggtcagtctcaggtccctccaggctttagatttcacccaacagaagaagagcttcttcactactacctcaggaagaaagttgctaatgagaagatagaccttgatgtaattcgcgaggttgatcttaataagcttgagccatgggacatccaagagaagtgcaaaataggatctacaccacagaatgattggtatttctttagtcacaaagacaagaaatatcccacagggactagaacaaatcgagctacggctgctgggttttggaaagccactggccgtgataagatcatctatagtgggtttaaaagaattggattgagaaagactcttgtgttttacagaggaagagctccacatggacagaaatccgattggatcatgcatgaatataggcttgatgacagcaccaacgacactaatgtctcaaatcctataggagaggcaatccctgaagaagggtgggtggtttgccgggtatttagaaagaagaactatcaaaaaacccttgagagtcccaaaagctcatcatgctcattggattcaaaggctcatcagattcttggttcaggaaatgatggagttcttgatcaaatacttctctatatgggaaggacttgcaagatggagaatgaaacatttagcaacatgaatatctccaacaacaacagtagtttaaggtttctctcagacaatagcatcagtgatgggctccatgaaagattcatgcaccttcctcggttagacagcccaccactcccttctattccactaagcagtccatcttttgatcaagatcgaagtttcaaatcttgttatcaccaatcgtacgatgagatgctgacagagaatgaaccttcctcttcaaaccaaattggcaatggcactttcgacatgatctcatcatccgtaattcatggctccaaatccgggcaacttaacgattgggtaactcttgatcgtctagtggcatcacaacttaatggacatgaagcagagacatccaagcatttatcttgctttactaccggcccaaatgcgagttttggtctttctcctgatgatgacatgcaattatcacacttgcaaaattctcatagatcatcatcaaacattcaagcaaatacttctcatgtgtataccaacgagaatgacctatggggcttcactaaatcttcgtctccatcatcatcatcggacccattatgccacttatcggtataacaaaagtgctcaatattgtataccctatagagtaatatagaaaccctaagagtctaggtacgtatacgttatatagtagt

SEQ ID NO: 70 - protein, 418 aa, Populus trichocarpa
MPEDMMNLSINGQSQVPPGFRFHPTEEELLHYYLRKKVANEKIDLDVIREVDLNKLEPWDIQEKCKIGSTPQNDWYFFSHKDKKYPTGTRTNRATAAGFWKATGRDKIIYSGFKRIGLRKTLVFYRGRAPHGQKSDWIMHEYRLDDSTNDTNVSNPIGEAIPEEGWVVCRVFRKKNYQKTLESPKSSSCSLDSKAHQILGSGNDGVLDQILLYMGRTCKMENETFSNMNISNNNSSLRFLSDNSISDGLHERFMHLPRLDSPPLPSIPLSSPSFDQDRSFKSCYHQSYDEMLTENEPSSSNQIGNGTFDMISSSVIHGSKSGQLNDWVTLDRLVASQLNGHEAETSKHLSCFTTGPNASFGLSPDDDMQLSHLQNSHRSSSNIQANTSHVYTNENDLWGFTKSSSPSSSSDPLCHLSV

SEQ ID NO: 71 - DNA, 1244 bp, Populus trichocarpa
ttcctcactccctcattaatgacagaaaacatgagtatatctgtgaatgggcaatctcaggtccctcctggctttcgatttcaccccacagaagaagagctcttggattactacttgaggaagaaggtctcgtatgagaagattgacttagaagtgatccgagatgttgatcttaataagctcgaaccatgggatatacaggagagatgcaaaataggaaccgccccacaaaacgattggtacttctttagccacaaggacaagaaatatccagccggcactcgcaccaatcgagcaactgctgcgggattttggaaggctactggccgtgacaaggtgatatacggcactggcaagcgggttggaatgaggaagactctcgtgttctacaaaggccgagccccacatggacaaaaatccgattggattatgcatgaatataggctggacgataatcccagtgaaaccaatgtctccaatgttatgggagaggcagcacaggaagatggctgggtggtttgccgtatcttcaagaagaaaaatctcaacaaaaccctagacagagctatgagttcatcacccatcactgcagataccaggaaccagacgttaagttcttgcaatgagggctctctagatcaaatgcttcattacatgggaagtacttgcaaagaagaaaatgaagcagataatagtgctagatatctttggcctattgacacagcaatcaaccatgtccaccatgataatagattcatggaacttccaagcttagagagtccaaactctaccagtagcctaaacttttaccaatccatgatcacagaaaatgaaggttcaataactaaccaggtgagttaccccttagactctggccttaacaactgggttgcccttgatagtctagttgcttctcaaatcaatggccaggctgaaacctctaggcaatcagcgtgcttccccaatgaccccaccatgacttattgtacccctactgatcttcatcatgatctccaattcccaaccctacgatcatcattttcgtttccatcaaacagatcttaccatgaaactcaagattataacagtgaaatcgacctctggaatttttccagtagatcatcgtccgacccacaatggcacttgccaaacactagtgtatagcagaattaccctacaaataaacattctatagttccaaaagacagaagagtattatgatgacggcagtagttaattatata

SEQ ID NO: 72 - protein, 381 aa, Populus trichocarpa
MTENMSISVNGQSQVPPGFRFHPTEEELLDYYLRKKVSYEKIDLEVIRDVDLNKLEPWDIQERCKIGTAPQNDWYFFSHKDKKYPAGTRTNRATAAGFWKATGRDKVIYGTGKRVGMRKTLVFYKGRAPHGQKSDWIMHEYRLDDNPSETNVSNVMGEAAQEDGWVVCRIFKKKNLNKTLDRAMSSSPITADTRNQTLSSCNEGSLDQMLHYMGSTCKEENEADNSARYLWPIDTAINHVHHDNRFMELPSLESPNSTSSLNFYQSMITENEGSITNQVSYPLDSGLNNWVALDSLVASQINGQAETSRQSACFPNDPTMTYCTPTDLHHDLQFPTLRSSFSFPSNRSYHETQDYNSEIDLWNFSSRSSSDPQWHLPNTSV

SEQ ID NO: 73 - DNA, 1070 bp, Populus trichocarpa
ggacaaaaatcgatactagatttagggagaatgatgacaggaaacggacaactatcagttcctccaggtttcagattccatccaacagatgaagagcttctgtactattatctgaggaagaaggtctcttatgaagccattgacctagatgttatcagggaggtggacctcaataaacttgagccatgggatctcagagataaatgcaagattggttcgggtcctcaaaatgagtggtattttttcagtcacaaggacaagaaatatccaaccggaacccgaacaaatcgggcaaccacagcaggtttttggaaagcaacaggaagggataaggccatccatattagcaactctcagaggatcggtatgagaaaaaccctagtcttctacactggacgtgctcctcatggtcaaaagacagattggatcatgcatgagtaccgcttggacgatgacaactctgaggttcaggaagatggttgggttgtctgcagggttttcaagaagaagaatcaaaccagaggtttcctaccagaagtttctcaagaagtacacttctctcacatgaaggccagcgcttcttcttttccactagatccaaaacaaaaccacttgcaatcactatatcaagactatacccttgatggatccatgcatctaccacagttatttagtccagactcagctgttgccccatcgtttgtgtcacccctctctttgaacaacatgaacgacattgagtgctcacagaacttgttgaggttaacaccgagtggttgtggatttgtgcaccctgcagggaggttcaatggtgattggtcatttctggacaagcttcttgcttctcatcaaaacctggatcatcagcagcactatcaaaacaaaaggaattcatcatcccaaattgttgatcctgtgggtacttcaactcagaaattcccatttcaacaccttggcttcgagactgacattctgagattttccaagtagagttgcaactcccatgtcattaaccgtgcttcattttatcttcttaattaaactggagggagcacgcatttttattcata

SEQ ID NO: 74 - protein, 319 aa, Populus trichocarpa
MMTGNGQLSVPPGFRFHPTDEELLYYYLRKKVSYEAIDLDVIREVDLNKLEPWDLRDKCKIGSGPQNEWYFFSHKDKKYPTGTRTNRATTAGFWKATGRDKAIHISNSQRIGMRKTLVFYTGRAPHGQKTDWIMHEYRLDDDNSEVQEDGWVVCRVFKKKNQTRGFLPEVSQEVHFSHMKASASSFPLDPKQNHLQSLYQDYTLDGSMHLPQLFSPDSAVAPSFVSPLSLNNMNDIECSQNLLRLTPSGCGFVHPAGRFNGDWSFLDKLLASHQNLDHQQHYQNKRNSSSQIVDPVGTSTQKFPFQHLGFETDILRFSK

SEQ ID NO: 75 - DNA, 1067 bp, Populus trichocarpa
aacaacagatactagatttaagggagaatgatggcaggaaacggacaactatcagttcctccaggtttcagattccatccaacagatgaagagcttctgtactattatctgaggaagaaggtctcgtatgaagccattgacctggatgttatcagggaggtggacctcaataaacttgagccatgggatctcaaagataaatgcaggattggttcgggtcctcaaaatgagtggtatttcttcagccacaaggacaagaaatatccaaccgggactcgaacaaaccgggcaaccaccgcaggcttttggaaagcaacgggaagggataaggccatccatcttagcaactctcagaggattggtatgagaaaaaccctagtcttctacactggacgtgctcctcatggtcaaaagactgattggatcatgcatgagtaccgcttggatgatgacaactctgaggttcaggaagatggctgggttgtctgtagggttttcaagaagaagaatcaaactagaggtttcctaccagaagtttctcaagaagaacacttctctcaaatgaaggctagcgcttcttctcttccactggagccaaaacaaaaccatatgcaatcaatttatgatgactatacctttgatggctccatgcatctcccacagttatttagtccggaatcagctgttgccccatcatttgtatcacctctctctttgaacaacatggacgacattgagtgctcacagaacttgttgaggttaacatctactggttgtggacttgtgcagcctgcagggaggttcaatggtgattggtcatttctggataagcttcttgcttctcaacaaaacctggatcatcatcagcactaccaaaacaaaggcaattcttcatcccaaattgttgaccatgtgggtgcttcaactcagaaattcccatttcaataccttggctttgagactgacattctgagattttcgaagtagagttgcgacgttccaagtcattaaccgtgctgcatcttatctccttaaactggagggagcatgtatttttattcataaca

SEQ ID NO: 76 - protein, 319 aa, Populus trichocarpa
MMAGNGQLSVPPGFRFHPTDEELLYYYLRKKVSYEAIDLDVIREVDLNKLEPWDLKDKCRIGSGPQNEWYFFSHKDKKYPTGTRTNRATTAGFWKATGRDKAIHLSNSQRIGMRKTLVFYTGRAPHGQKTDWIMHEYRLDDDNSEVQEDGWVVCRVFKKKNQTRGFLPEVSQEEHFSQMKASASSLPLEPKQNHMQSIYDDYTFDGSMHLPQLFSPESAVAPSFVSPLSLNNMDDIECSQNLLRLTSTGCGLVQPAGRFNGDWSFLDKLLASQQNLDHHQHYQNKGNSSSQIVDHVGASTQKFPFQYLGFETDILRFSK

SEQ ID NO: 77 - DNA, 1379 bp, Populus trichocarpa
gctttatacaataaatttgtcctagcaaaaatgcctgaagatatggtgaatctatcgatcaatggtcagtctcaggttcctccaggctttagatttcacccaacagaagaagagcttcttcactactacctgaggaagaaagttgcttatgagaggatagaccttgatgtaattcgtgaggttgatcttaataagcttgagccatgggatatccaagagaagtgcaaaataggatctacacctcagaatgactggtatttcttcagtcacaaagacaagaaatatcccacagggacaagaacaaatcgtgctacggctgccggattctggaaagctactggccgtgataagatcatctatagcgggtttaaaagaattggattgagaaagactcttgtattttacaaaggaagagctccacatggtcagaaatccgattggatcatgcatgagtataggcttgatgacagcacccatgaaactaatgtttcaaatcctataggagaggcaatcccagaagaaggttgggtggtttgccgggtatttagaaagaagaactatcaaaaaacccttgagagtcccaaaggctcatcaagctcattggattcaaaggctcaccagattcttggttcagggaatgaaggagttcttgatcagatacttatgtacatgggaaggacttgcaagatggagaatgaaacatttagcaacatgaatatctccaacaacagcaacagcagtagtttaaggtttctatcgaacaactgcatcagtgaaggactccatgaaagattcatgcaccttcctcggctagaaagcccaacactcccttctattccaatcagaagtccatctttcgatcaagatcgaagtttcatttcttgttatcatcaatcatacgatgagatgctgacagagaatgaaccttcctctccaaaccaagttggcaatggcatcttcgacatgacctcatcctcaatgactgatcatgactccaaatccgggcaacttaatgattgggtaactcttgatcgtcttgtggcatcacagcttaatgggcatgaagtagagacatccaagcaattatcttgctttagtactgacccaaatgcgagttttggtctttctcctgatgatggcatgcaattatcacacaaacagcattctcatggatcatcatcatcaaacattcaagcaaactcttcccatgtttacagcaacgagaatgacctgtggagccttactaaatcatcatctccatcttcatcatccgacccattatgccacttgtcggtataacaaaagtgctcgatattttataccctattgaataatatagaaaccctgaaaatctaggtatgtatacgtacgttatagtc

SEQ ID NO: 78 - protein, 422 aa, Populus trichocarpa
MPEDMVNLSINGQSQVPPGFRFHPTEEELLHYYLRKKVAYERIDLDVIREVDLNKLEPWDIQEKCKIGSTPQNDWYFFSHKDKKYPTGTRTNRATAAGFWKATGRDKIIYSGFKRIGLRKTLVFYKGRAPHGQKSDWIMHEYRLDDSTHETNVSNPIGEAIPEEGWVVCRVFRKKNYQKTLESPKGSSSSLDSKAHQILGSGNEGVLDQILMYMGRTCKMENETFSNMNISNNSNSSSLRFLSNNCISEGLHERFMHLPRLESPTLPSIPIRSPSFDQDRSFISCYHQSYDEMLTENEPSSPNQVGNGIFDMTSSSMTDHDSKSGQLNDWVTLDRLVASQLNGHEVETSKQLSCFSTDPNASFGLSPDDGMQLSHKQHSHGSSSSNIQANSSHVYSNENDLWSLTKSSSPSSSSDPLCHLSV

SEQ ID NO: 79 - DNA, 1199 bp, Populus trichocarpa
caggactcaagacgtacgcagcaggagaatatgaatacttttacacatgttcctcctggtttccggttccatcctaccgacgaagaacttgtcgattactaccttaggaaaaaagtgaattcaagaaggattgatgtagatgtcattaaagatgtcgacctttacaaaattgagccatgggatcttcaagaactatgccgaataggaaccgaggaacaaaatgaatggtacttttttagccacaaagataagaagtatccaactggaactcgcacaaatagagccactgctgctgggttttggaaagcaacaggtagagacaaggcaatttattcgaagcaagacttgatcggaatgaggaagaccttagtcttttataaaggtcgagctccgaatgggcagaaatcggactggattatgcatgaataccgactcgaaacagatgaaaatgggactccacaggcaaaaggttgggtcgtgtgtagggtgttcaagaaaagattaccaacgatgcgaaaagtgagtgagcatgaatcagtctgttggtatgatgatcaagttgcattcatgcatgacttggattcaccaaagcaaaattctcagcctgatttgggttatcaattcccatacccctgcaagaaagagatagatctgcagtaccaaatccctcatgatcacttcctccaactcccacttctacaaagccctaaactgctgcaaccagctccaactataagctgcaattccatcaatgctgcatatggcctggacataaaccaggcaagcactatacaatcctcaacactcacacaagaagatcacattcaacaagcacatgagcaaagctttccctctatctatggtagcaacaacattaatgagcaagcagttgatcaagtgacagactggagagtacttgacaaatttgttgcttctcaattgagccaagaagatgtggtcaaggaaaacaaccacccaaatgccagcaacaacatcttcaacacatcatcaatccatgcaccaaacatatttgctcgacacttgagtaagcaagatacatccgtgccggaaaatgcctcgacgtcaacctccagttgtcaaattgatctatggaaatgatgatatccagttaaaaactacgctgcatggtatatactaatcataggatgtttcaatttgaatgcaaagggaaattatga

SEQ ID NO: 80 - protein, 362 aa, Populus trichocarpa
MNTFTHVPPGFRFHPTDEELVDYYLRKKVNSRRIDVDVIKDVDLYKIEPWDLQELCRIGTEEQNEWYFFSHKDKKYPTGTRTNRATAAGFWKATGRDKAIYSKQDLIGMRKTLVFYKGRAPNGQKSDWIMHEYRLETDENGTPQAKGWVVCRVFKKRLPTMRKVSEHESVCWYDDQVAFMHDLDSPKQNSQPDLGYQFPYPCKKEIDLQYQIPHDHFLQLPLLQSPKLLQPAPTISCNSINAAYGLDINQASTIQSSTLTQEDHIQQAHEQSFPSIYGSNNINEQAVDQVTDWRVLDKFVASQLSQEDVVKENNHPNASNNIFNTSSIHAPNIFARHLSKQDTSVPENASTSTSSCQIDLWK

SEQ ID NO: 81 - DNA, 1190 bp, Populus trichocarpa
gttcatcatccaaactggtatactagttgcatggctacatctacaggcggtgtgcctccagggttccgattccacccgacggaagaagaactgcttcattactacttgaaaaagaaggtttcttttcagaagtttgacatggaggtcattagagaggtggacttgaataagatggagccttgggagttacaagagacatgtaaaatcgggtcaacgccgcaaaacgagtggtattttttcagtcacagagacaggaaatacccaacagggtcgagaacgaatagggcaacaaacgcagggttttggaaggcaaccggaagggataagtgcatcaggaacaccgacaagaagattggtatgaggaaaacacttgttttctatcgaggaagagctcctcatggccaaaagactgactggatcatgcatgagtatcggcttgaagatggtgacgatgctcaaggaaacgctggtgaggatggttgggtggtctgcagagtgttcaagaagaagaatctattcaaggtcagcaatgaaggaggaaccgcaagcatggactcatctgaccaacaactccacacatcaagcaatcaatctcgaacttccatgtacagagacagccaatacttaattcgtcaacaccataaccatggtagcactcaacagacttttgaaccaaacaagcctgagctagccctttaccatcatcacatggaaacaccccatcaatattctctcttcccatcccaaaccctaatgccaactcataagcctttaggttatgactactcggctctcccttcggagtcacctgtcatggtcaagcaactcatgtcgaaagctagggattgtgagagtggaagtgaaagcctgaggtaccaagcttgtgagccagggttagaggtgggcacatgtgaagcacctcaacaaatggttgcaggaagagatgagcagggcatgaatgatcaatgggctgtgctcgtcacttctcatctaggaaatgaagattcttccaaaggggtgacgtttaatgccgcaaatgcaccatccgcgcacccaattaatcaatcctcacttcgtggggagatggatttttggggctatggaaaatagtggtactgtgttcttagttcctttcgtatttctttttggaagactacgtactacgaacatggattggaaccggtcatgaa

SEQ ID NO: 82 - protein, 359 aa, Populus trichocarpa
MATSTGGVPPGFRFHPTEEELLHYYLKKKVSFQKFDMEVIREVDLNKMEPWELQETCKIGSTPQNEWYFFSHRDRKYPTGSRTNRATNAGFWKATGRDKCIRNTDKKIGMRKTLVFYRGRAPHGQKTDWIMHEYRLEDGDDAQGNAGEDGWVVCRVFKKKNLFKVSNEGGTASMDSSDQQLHTSSNQSRTSMYRDSQYLIRQHHNHGSTQQTFEPNKPELALYHHHMETPHQYSLFPSQTLMPTHKPLGYDYSALPSESPVMVKQLMSKARDCESGSESLRYQACEPGLEVGTCEAPQQMVAGRDEQGMNDQWAVLVTSHLGNEDSSKGVTFNAANAPSAHPINQSSLRGEMDFWGYGK

SEQ ID NO: 83 - DNA, 1247 bp, Populus trichocarpa
atcctctctcgctcgcgaatgacagaaaacatgagtatatctgttaatggtcaatcccaggtccctcctggatttcgatttcaccccacagaagaagagcttttgcattactacttgaggaagaaggtctcgtatgagaagattgacttagacgtgatccgggatgttgatcttaataagcttgaaccgtgggatatacaagagagatgcaaaataggaaccaccccacaaaatgattggtacttctttagccacaaggacaagaaatatccaactggtacgcgcaccaatcgggcaactgctgctgggttttggaaggctactggccgtgacaaggtgatatacagcactggcaagcggattggaatgagaaagactcttgtcttctacaaaggccgagccccacatggacaaaaatccgattggattatgcatgaatataggctggacgatagcaccagtgataccaatgtctccaatgttatggaagaggaggcggcacaggaagagggatgggtggtttgccgtatcttcaagaagaaaaacctcaacaaaaccctggacaaaccttttagttcatcacccatcagtgcagatacaaggaaccagatgttaagttcttgcgatgaaggcactatagatcagacatttcattatatgggaaggacttgcaaagaagaaaatgtagcagacaatagtgctactgctagatatctcaggcctgttgacacagcaatcaactatgtccaccatgatggattcatgaagcttccaagcctagagagtccaaactctatcagtagccaaaattgctaccaacccatgatcacagacaatgaaggttcaataactaaccagatgagttaccccttggaccctggccttgacaactgggcaacccttgatcgtctatttgcttatcagcttaatggccagactgaaacctctaggcaattaccctgcattgacccaaccattacttattgcacccctagtactgatcttcatcatgatctccgattgccaaccttacgatcatcatttccgttaccatcaaacagatcttatcatggaactcaagattataacaacgagatagacctctggaattttaccactagatcttcccccgacacactatgccagttgtcaaacacaggtgcataacagaattaccctacaaataatcattctatagtccttaaagtcagaatagttttatgatgacagcagttttatatgtgtgg

SEQ ID NO: 84 - protein, 382 aa, Populus trichocarpa
MTENMSISVNGQSQVPPGFRFHPTEEELLHYYLRKKVSYEKIDLDVIRDVDLNKLEPWDIQERCKIGTTPQNDWYFFSHKDKKYPTGTRTNRATAAGFWKATGRDKVIYSTGKRIGMRKTLVFYKGRAPHGQKSDWIMHEYRLDDSTSDTNVSNVMEEEAAQEEGWVVCRIFKKKNLNKTLDKPFSSSPISADTRNQMLSSCDEGTIDQTFHYMGRTCKEENVADNSATARYLRPVDTAINYVHHDGFMKLPSLESPNSISSQNCYQPMITDNEGSITNQMSYPLDPGLDNWATLDRLFAYQLNGQTETSRQLPCIDPTITYCTPSTDLHHDLRLPTLRSSFPLPSNRSYHGTQDYNNEIDLWNFTTRSSPDTLCQLSNTGA

SEQ ID NO: 85 - DNA, 638 bp, Populus trichocarpa
gcccgatcaggagattgaagaatattgtcaatggagatggaatcctgtgtcccaccaggctttagatttcacccaacagaagaagaacttgtaggttactacctcaagaggaagattgactcacagaaaatcgatctagatgttatcgctaacattgatctctacaaaatggagccgtgggacatccaagccagatgcaatctagggtatgctgaacagaatgagtggtatttcttcagtcacaaggacaggaagtatccgaccggaacacggactaatagagccactgctgctggattttggaaggcaaccggaagggacaaggccgtgctctccaagaacaggcttatagggatgaggaagaccttagtgttctacaagggacgtgcacccaatggaaggaaaactgactggatcatgcatgaatatcgtctccaaacatctgaacatggaccccctcaggcaagctcattcttcattaattttaataataacaaaataaaaaataaaaaaatcatttgtctttctcaacattgcacgtataaaagcattccttaaacgctccttcttggagtgcagatatagctgtataaccaagcaagcattcacaccttgaaataattatagctagcaataaa

SEQ ID NO: 86 - protein, 175 aa, Populus trichocarpa
MEMESCVPPGFRFHPTEEELVGYYLKRKIDSQKIDLDVIANIDLYKMEPWDIQARCNLGYAEQNEWYFFSHKDRKYPTGTRTNRATAAGFWKATGRDKAVLSKNRLIGMRKTLVFYKGRAPNGRKTDWIMHEYRLQTSEHGPPQASSFFINFNNNKIKNKKIICLSQHCTYKSIP

SEQ ID NO: 87 - DNA, 1202 bp, Populus trichocarpa
gcaggactcaaggcctagcaggatcagaaaatgaattcttttacacacgttcctcctggttttcgattccatcctaccgatgaagaacttgtccattactaccttagaaaaaaagtaaattcaagaaggattgacctagatgtcattaaggatgtcgagctttacaagattgagccatgggatcttcaagaactgtgccgaataggaaccgaggagcaaaatgaatggtacttttttagccacaaagataagaagtatcctactgggactcgcacaaatagagccactgttgcaggattttggaaagcaacaggtagagacaaggcaatttattcgaagcacgacttgatcggaatgaggaagaccttagtcttttataaaggtcgagctccgaatggacagaaatcggactggattatgcatgagtacagacttgaaacagatgaaaatgggactccacaggcaaaaggttgggttgtgtgcagggtgttcaagaagagaataccaacgatgccaaaagtgagtgagcatgaatcagtttgctggtatgatgatcaagttgcattcatgcatgacttggattcaccaaagcaaaacttccagcctgacttaggttatcaattcccttactcctgcaagaaagagctggattttcagtaccaaatccctcatgatcacttcctccagctcccacttctacaaagtcctaaacttctgcaatcggctcctacattaagttgcaattccatcaatgctgcatatggcctagacataaaccagacaagcacttttcaatcctcaacactcacacaagaagatcacgttcaacaaacacatgagcaaagatttcccaccatctatggtaacaacaacagcaatcatgagcaagcagttgatcaagtcacagactggagagtgcttgacaaatttgttgcttctcaattgagccacgaagatgtgatcaaggaaactaaccacccaaatgctagcaacaacattttcaacacatcatcagtccacgcagcaaacatacttgttagacacttgaataagcaagaagcatccgtgcaggaaaacgcctcgacgtcaacctccagttgtcaaattgatctatggaagtgatgatatccagccaaaatctatgattcatggcataaactgatcataggatgtttcagtttgaaagccaagggaaattatta

SEQ ID NO: 88 - protein, 363 aa, Populus trichocarpa
MNSFTHVPPGFRFHPTDEELVHYYLRKKVNSRRIDLDVIKDVELYKIEPWDLQELCRIGTEEQNEWYFFSHKDKKYPTGTRTNRATVAGFWKATGRDKAIYSKHDLIGMRKTLVFYKGRAPNGQKSDWIMHEYRLETDENGTPQAKGWVVCRVFKKRIPTMPKVSEHESVCWYDDQVAFMHDLDSPKQNFQPDLGYQFPYSCKKELDFQYQIPHDHFLQLPLLQSPKLLQSAPTLSCNSINAAYGLDINQTSTFQSSTLTQEDHVQQTHEQRFPTIYGNNNSNHEQAVDQVTDWRVLDKFVASQLSHEDVIKETNHPNASNNIFNTSSVHAANILVRHLNKQEASVQENASTSTSSCQIDLWK

SEQ ID NO: 89 - DNA, 1434 bp, Solanum lycopersicum
ctaaattccttcttgtttatcattttctctcttcccaaaaaaaaaatcccaaaatttaatcataatacaattcgaatttatcaacctcgtactacgtacatatttttgttggtacgtaaaatactgaattcaggtcaactcaaacatcgtaaattgtgatttctttatggaaagtacggattcatcaaccgggacacgtcatcagcctcaactcccaccggggtttcgattccacccgacggacgaagaactcatcgtccactacctcaaaaaacgagtcgccggcgctccgattccggtggatattattggtgaaattgatctttataagtttgatccatgggaactccctgctaaggcaatattcggagagcaagaatggttcttttttagtccaagagatagaaaatatcctaacggggcgaggccaaatcgggctgcaacatcgggttattggaaggctaccggaaccgacaagccggtttttacttccggtggaacacaaaaggttggggtaaaaaaggcgctcgttttttacggcggtaaaccaccaaaaggggtaaaaactaattggatcatgcatgaatacagagttgtagaaaataaaacaaataacaagccacttggttgtgataatattgttgccaacaaaaaaggatctttgaggctagatgattgggttttatgtcgaatttacaagaagaataacacacaaaggtccatagatgatttgcatgatatgttgggatcgataccacaaaatgtaccaaattcaatattacaaggaataaagccttcaaactatggtacaatattgctcgaaaatgaatcgaatatgtacgatggaattatgaataacacgaacgatattatcaacaataataatagatccattccacaaatatcgtcaaagagaacgatgcatggaggtttgtattggaataacgacgaagcaacaacaacaacaacaactattgataggaaccattctccaaatacaaaaaggttccttgttgagaacaacgaggacgatggacttaacatgaataatatttcgcgaattacaaatcatgaacaaagtagctccattgccaatttcctgagccagtttcctcaaaatccttcgattcaacaacaacaacaacaacaagaagaagtattgggatctcttaatgatggggtcgtctttcgacaaccttataatcaagttactggcatgaattggtactcttaaagatataaaaaggcaaaaaatagttagccctgtaaaatcaatcgatcaatcaatcatagatatattatatatggatttcgttatattttacttttagttagaattaatatatagaatatcttctatctcacattaacaaataagaacatttataacaaatagtgaaactaaactatctaccattagatagacattttgtt

SEQ ID NO: 90 - protein, 355 aa, Solanum lycopersicum
MESTDSSTGTRHQPQLPPGFRFHPTDEELIVHYLKKRVAGAPIPVDIIGEIDLYKFDPWELPAKAIFGEQEWFFFSPRDRKYPNGARPNRAATSGYWKATGTDKPVFTSGGTQKVGVKKALVFYGGKPPKGVKTNWIMHEYRVVENKTNNKPLGCDNIVANKKGSLRLDDWVLCRIYKKNNTQRSIDDLHDMLGSIPQNVPNSILQGIKPSNYGTILLENESNMYDGIMNNTNDIINNNNRSIPQISSKRTMHGGLYWNNDEATTTTTTIDRNHSPNTKRFLVENNEDDGLNMNNISRITNHEQSSSIANFLSQFPQNPSIQQQQQQQEEVLGSLNDGVVFRQPYNQVTGMNWYS

SEQ ID NO: 91 - DNA, 1663 bp, Solanum lycopersicum
  misc_feature (2)..(2): n is a, c, g, or t
cnttncggccgggganantaaggcatgttattataatccacaaaatcactttgtccctctataaatggctaatctttcttggattttcgatttccgtgtcaagcaagccttcgccattttcactttacttcacttgtaatcttcacaattctcatatctatgataccctttatctttatgttccatgtatttttcttaattgggtattctcaaaaaagttaaatttacattcttggctttttgggtggttgagaaatggaacaagaaggatcgaaaggggctgtaacggtggcaccaacgtctttagcaccgggattcaggtttcatcctaccgatgaggaattagttaggtactatctacgccgaaaggtctgtggaaagccattcagatttcaagctgttactgaaattgatgtgtacaaatctgaaccttgtgaccttgcaggatattcatcggtgaagagcagagatatggagtggtacttcttcagccctgtggataagaagtattgtaatggatctcggcttaaccgagctactggtcaaggatattggaaagctactgggaaagatcgtcatatcagccataagtctcaggccattgggatgaagaagactctcgttttccatagtggtcgagctcccgatggcaagagaactaattgggtaatgcatgagtacaggcttgcggacaaagagttggagaaggctggaatcgtgcaggattcctttgttctatgtagaatcttccaaaaaagcggtttgggaccaccaactggtgacagatatgcaccatttatcgaggaggaatgggatgacgattcagccgtgcttcctggaggagagacagaggatgacgcagtgaatggtgttgatgcacgagttgagagcaacgatcttgaccaggatgctccgcccaagattgcttgcgataatgagaacgtgattgaacttcgaagtcttccatttgcttgcaagagggagagatcagaagaacctgaacttctctctttaagccaaaacaagaaaccgaagcatgatgttccaagctctagccgtgcaaatggttctgaagactcaactattactagtaaagatccggagaatatgatgacgacaaaagattaccctctagctctcttaggattcccgttgttagagtccttcgagcccaaggatccccaaccttctaatcccctcacatttgattcctccaatcttgaaaaatccgtccctccaggctacctgaagttcatcagcaatctagagacggagatcctaaatgtctccatggagagggagacgctaaagattgaagtaatgagagctcaagccatgatcaacgttcttcaatcgcggattgatctcttgaccaaagagaacgaagacatgagaaaacttgtccgaggtggttagacaataacagcatttcgattcggtttaggtttgtactaccttcaggaagagcaagatatagtaaaaagatgtttgaagacatttctgtagttcttgttgtaacctggatcatatgtacctttttaggtatctgcagaatttttttctattgcattagtttaaactttgaagtcttagactgttatcttaatgatattgttgtttaataacaatagtcgggtcatgttgtgcaaaaaaaaaaaaaaaa

SEQ ID NO: 92 - protein, 386 aa, Solanum lycopersicum
MEQEGSKGAVTVAPTSLAPGFRFHPTDEELVRYYLRRKVCGKPFRFQAVTEIDVYKSEPCDLAGYSSVKSRDMEWYFFSPVDKKYCNGSRLNRATGQGYWKATGKDRHISHKSQAIGMKKTLVFHSGRAPDGKRTNWVMHEYRLADKELEKAGIVQDSFVLCRIFQKSGLGPPTGDRYAPFIEEEWDDDSAVLPGGETEDDAVNGVDARVESNDLDQDAPPKIACDNENVIELRSLPFACKRERSEEPELLSLSQNKKPKHDVPSSSRANGSEDSTITSKDPENMMTTKDYPLALLGFPLLESFEPKDPQPSNPLTFDSSNLEKSVPPGYLKFISNLETEILNVSMERETLKIEVMRAQAMINVLQSRIDLLTKENEDMRKLVRGG

SEQ ID NO: 93 - DNA, 2140 bp, Solanum lycopersicum
ggtcccaattcgattcctaatccatttttttctctcaagatcatattttttggatacccttttgcttattgaatcattttgtggttgaatttttttttattagtgaagagatgaagatgtttgagttatctgattcggattcgtttgctgtttttgcatcatcaaagaatgtttttccaccgggatttaggtttcatccgactgatgaagaacttgttctgtactatttgaagaagaagatctgcgggaagaggattctgcttgatgccattgctgagactgatgtttacaaatgggaaccggaggacttgcctgatctatctaagttaaaaactggggaccgccagtggttcttctttagccccagagatcgaaagtatcctaatggagcacgggcaaacaggggaacaaagcgtgggcactggaaagttacaggaaaggatcgaatcattatgtgcaactctcgtgctgttggacttaagaagaccctagtcttttataaaggccgagcaccggtaggtgaacggacagactgggtgatgcatgagtatactatggatgaagaagagctgaaaaaatgtcaaaacgcccaggactactatgcgctttacaaggtatttaagaagagtggctccgggcctaagaatggtgagcagtatggtgcaccttttagagaagaggagtgggctgatgatgaatgtcctagtgccaaaggctttgtccatcaggataattccactaaccacgttattgaagctccttctgtggatggtggacttgaggagttgttgaaccccgtgcttatggagccactttctgtagattatgattatgcagtggagcagcttgtacaggaggaagatactcgaagtaccttgctatatcattccgcaaaggaagtcaattttccccaccgcagtgcggtgattgccccagcaaaggaaagctttgacttgacacagtcaggcacatcacagcgacagttgcatgaggcactagaagtcacatctgctcctgttatttatgaacagcagccacatgtggtggaagaggattttctggagatggatgaccttcttggcccagagccaagcactcagaactttgataacttggggccgtgtgttcaaaactttgatgaggcaggccaaagtggtcaaaactttgatatgcctgctggaaacttcgaagatttgccattcgattattttgatgggttggcagagtttgacctgtaccatgatgcatccttgcttgatgatgtaagaacaactgaagtggggcaaaatactgaaccacacatgaacaacttagtgaatggttctgttaaccctgcttctacaacgtatataaacactttccagcatgaaatgatgaacaaccagccaatgtatttgaataattccgaacagattagcaatcaactgtgggtacacgatcaaaggttcaatatctcttaccccagcgaggcaaatcagttggttgctcctccagctacttcagatgatggtataagaataaccatgtcatcttcagcatggcgagaactctattgcccaagtggaaatgggggagtttgagcctgcaagagctctgctgctgctaatatattggtgttgagattatttttttcccctaatttttgcaagttttggcaatgttttaggtgttgtatatgatagtattttggcaaatcatcctatggacgcaaatcaaaatcaaccacccaatcaagatgaaggtacaccatcatggttaacctctcagttgtgggccttcgtggattctatacctactgctcctgctatagctgctgaaagtcctgtggttaatagtgcctttaagcgcatgtctagctttagtaagatgagaataaacgccaggaacatgaatgttgcggcagcaggtaacacagcaacttcaagaagttcgcgctactctaagaatgggcttttttatttttcttttgttggaatactgtgtgcaatcttatgtgtattactaggaacatttgtcgacatcttgaggagacccatatcctcttaaatatgcaaagaaaagttgatgttctatcatttggcatcttgccagaatcaatgagaagcagagcatacattttatgagcaaaaaaaaaaaaaaa

SEQ ID NO: 94 - protein, 489 aa, Solanum lycopersicum
MKMFELSDSDSFAVFASSKNVFPPGFRFHPTDEELVLYYLKKKICGKRILLDAIAETDVYKWEPEDLPDLSKLKTGDRQWFFFSPRDRKYPNGARANRGTKRGHWKVTGKDRIIMCNSRAVGLKKTLVFYKGRAPVGERTDWVMHEYTMDEEELKKCQNAQDYYALYKVFKKSGSGPKNGEQYGAPFREEEWADDECPSAKGFVHQDNSTNHVIEAPSVDGGLEELLNPVLMEPLSVDYDYAVEQLVQEEDTRSTLLYHSAKEVNFPHRSAVIAPAKESFDLTQSGTSQRQLHEALEVTSAPVIYEQQPHVVEEDFLEMDDLLGPEPSTQNFDNLGPCVQNFDEAGQSGQNFDMPAGNFEDLPFDYFDGLAEFDLYHDASLLDDVRTTEVGQNTEPHMNNLVNGSVNPASTTYINTFQHEMMNNQPMYLNNSEQISNQLWVHDQRFNISYPSEANQLVAPPATSDDGIRITMSSSAWRELYCPSGNGGV

SEQ ID NO: 95 - DNA, 1201 bp, Solanum lycopersicum
atctttcaaccgttttcctattccttcaattctattcaaagaattcagaaaactctgtctttagactgaaataagaaatttatatttaaaagaaaaagaaaaaatgacagcagctgaattgcagttacctccaggattcagatttcatccaactgatgaagaacttgtgacgcattatttatgcagaaaatgcacttcacagccaattgctgtaccaattgtagcagaaattgatctctacaagtttgatccatgggatcttcctgatttggcattgtatggagaaaaggagtggtacttcttttcacctcgagatcggaagtatccgaatggttcacgacctaatcgagctgctggaactggatattggaaggctactggtgcggataagccgattggaaatccgaaagcggtgggaattaagaaagcattggtgttttattctggtaaagcacctaaaggagagaaaactaattggatcatgcatgaatatcgtctagctgatgttgatcgatctgctcggaagaataacaatagcttaaggctggacgattgggttctatgtagaatttacaacaaaaagggatcgattgagaagaacaggaaaatgaacactacctgctacatggataaagttggatctccagaagatcggaagccggagattctaccaccgctacctccacatcctcagccgcagcaattgcacaacgatttcttctacctgccgtccgattcagttccgaagatgcactccgattcaagctgctcggaacacgtgctctcacccgaattcacatgtgaaagagaagtgcagagtgagccgaaactgacagattgggagaaagctaccctcgatcttccatttaattacatggatgccactaccggcgccacaacggtggataatagcttgttaggttctcagtttcagagcagttatcagatgtcgccgctgcaggatatgttcatgcacctgcacaagcctttttgagtggggccaccaatggattattttcagccgcaagtgtcaaacgagactttaacgtccgtgactttgcacgtgccgccgtagtagaataataggagtagcatcttctgcgttcacattggcaaattgtgaaaacaacatttgtacatgtagtagatgatggggaaaaaaaatattattattagcaattaaaataaaattgcaaatatg

SEQ ID NO: 96 - protein, 296 aa, Solanum lycopersicum
MTAAELQLPPGFRFHPTDEELVTHYLCRKCTSQPIAVPIVAEIDLYKFDPWDLPDLALYGEKEWYFFSPRDRKYPNGSRPNRAAGTGYWKATGADKPIGNPKAVGIKKALVFYSGKAPKGEKTNWIMHEYRLADVDRSARKNNNSLRLDDWVLCRIYNKKGSIEKNRKMNTTCYMDKVGSPEDRKPEILPPLPPHPQPQQLHNDFFYLPSDSVPKMHSDSSCSEHVLSPEFTCEREVQSEPKLTDWEKATLDLPFNYMDATTGATTVDNSLLGSQFQSSYQMSPLQDMFMHLHKPF

SEQ ID NO: 97 - DNA, 1574 bp, Solanum lycopersicum
taatattttttttatgaaaaaaaaagaagttttttattaattaaaaaaaaaattatgggtgttcaagaaaaagatccacttttgcaattaagtttaccaccaggatttagattttatccaactgatgaagagcttttagttcaatatttatgtaagaaagttgctggacatgattttcctctacaaattattggtgaaattgatttatacaaatttgatccttgggttttacctagtaaggcgacatttggtgaaaaagaatggtatttcttcagtccgagggataggaagtatccgaatggatctagaccgaatcgagtagcaggttcgggttattggaaagcaacggggacggataaggtgataacttcgcaaggaagaaaagttggaattaagaaagctcttgtgttttatgtgggtaaagctccaaaaggatccaagacgaattggattatgcatgaatatagactttttgaatcttcaaggaaaaataatggaagttcaaagctagatgaatgggtgctttgtcgaatttataagaagaattcaagtggaccaaaacctctgatgtctggtttacacagcagtaatgaatacagccatggttcgtcgacttcgtcatcatcccaattcgatgatatgctcgaatcattaccagaaatggacgatcgattctccaatttaccgaggttgaactctctcaaggccgagaaattcaacctcgatcgtctggattcagccaatttcgattgggcaattctcgctgggctcaaaccaatgccggaattgggcccagcaaatcaagctccaggcgttcagggtcaggctcaggggcatgtcaataaccacattcacagcgacaacaacaatatgaattttctcaacgatgtttacgcccatcctccaaatttccgaggcaacacaaaggttgaaagtattaatctagacgaagaagttgaaagcgggaaaagaaatcaacggattgaccaatcgagttacttccaacagagtctcaatggattttcccaagcgtatacaaacaatgtggatcaattcgggatccaatgtccgaatcagacgttaaatctcgggttcaggcagtagaggatactggaaagtaagaaaagttgtagctcgaaatctgaatgccagtatactgtgtatatatacagaaggcctggatttagatttccttggcccaacggattcgaagaaagagcaagtacagtatcatgccaaggaaattaagtccttcgagggcacgtctgggcagacgtcgtggacgttatttgcaagttcttgcaattgtgtctgaagtcattaggctgagtggcacttcttatttgcaagttgtgtttgaagtcattaggccgaaggcacgtctgcgtggacgataaaataatgtacacaaacaggagatatatgcaggggcgtgtatattcttttaggaaactggcttttagtttgttttattttatgatacatttccagatttggatgtattttgtaatttttacatttctgtgtagcaagttagaagatatatagaaagatacttttaaaatactttttat

SEQ ID NO: 98 - protein, 349 aa, Solanum lycopersicum
MGVQEKDPLLQLSLPPGFRFYPTDEELLVQYLCKKVAGHDFPLQIIGEIDLYKFDPWVLPSKATFGEKEWYFFSPRDRKYPNGSRPNRVAGSGYWKATGTDKVITSQGRKVGIKKALVFYVGKAPKGSKTNWIMHEYRLFESSRKNNGSSKLDEWVLCRIYKKNSSGPKPLMSGLHSSNEYSHGSSTSSSSQFDDMLESLPEMDDRFSNLPRLNSLKAEKFNLDRLDSANFDWAILAGLKPMPELGPANQAPGVQGQAQGHVNNHIHSDNNNMNFLNDVYAHPPNFRGNTKVESINLDEEVESGKRNQRIDQSSYFQQSLNGFSQAYTNNVDQFGIQCPNQTLNLGFRQ

SEQ ID NO: 99 - DNA, 1286 bp, Solanum lycopersicum
ggcacgagcaaagcaggagcaggagcagcaacaaacagagagaagaaaacagaggaagataagaggaaaatttatcgaattcgaatcgagagaaaaggggaagtgaagttgcgaagagtgagaatttcaaaggaaatgaacaaaggagcaaacggaaatcagcaattggagttaccggcgggattcagattccatccgacagacgacgaattggtgcagcactatctctgcaggaaatgcgccggacagtcgattgctgtatcaattatagctgaaattgatctttacaagtttgatccatggcagttgcctgagaaggctttgtacggtgaaaaagagtggtattttttctcaccaagggatagaaaatatccgaacggttcacggccgaaccgagcagcaggaaccggttattggaaggcaaccggagctgataaaccggtgggaaaacccaaaaccttagggataaagaaggcacttgtgttctatgccggaaaagcacccagaggtataaaaacaaattggattatgcacgagtaccgcctcgccaacgtggaccgctctgctggcaagaacaataacttgaggcttgatgattgggtattgtgtcgaatatacaacaagaaaggcacacttgagaagcattacaatgtggacaacaaggaaactacaagctttggagaatttgatgaagaaataaaaccaaaaatattgcccacacaattagcaccgatgccaccacggcctcgatcgacaccagcaaacgactacttttatttcgagtcatcagagtcgatgactagaatgcacacgacaaactcgagctctggctcagagcatgtcttgtcgccatgtgacaaggaggttcagagcgcgcccaaatgggacgaagaccacagaaacacccttgattttcagctaaactatttggatggtttactaaatgaaccatttgaaacccaaatgcagcagcaaatttgcaactttgaccagttcaacaatttccaagacatgttcctatacatgcaaaaaccttactaaaattgtataaattcattggatctaaattgagtgtgatccatgacattttctttgttctttggtggtgtaggtcaactttttattaagtagtttagagaagtacaaaatgctagtcaaatttggtgggctacagcacaaatgagccttgataagcatagccaaagagtcgtatagaagggcttattattattgtaaggtatgtaaaaacaaatgaaaatttgttaatatcaagttatcattcttct

SEQ ID NO: 100 - protein, 301 aa, Solanum lycopersicum
MNKGANGNQQLELPAGFRFHPTDDELVQHYLCRKCAGQSIAVSIIAEIDLYKFDPWQLPEKALYGEKEWYFFSPRDRKYPNGSRPNRAAGTGYWKATGADKPVGKPKTLGIKKALVFYAGKAPRGIKTNWIMHEYRLANVDRSAGKNNNLRLDDWVLCRIYNKKGTLEKHYNVDNKETTSFGEFDEEIKPKILPTQLAPMPPRPRSTPANDYFYFESSESMTRMHTTNSSSGSEHVLSPCDKEVQSAPKWDEDHRNTLDFQLNYLDGLLNEPFETQMQQQICNFDQFNNFQDMFLYMQKPY

SEQ ID NO: 101 - DNA, 1119 bp, Solanum lycopersicum
ctcaatattattattttcctcttacatagaatcaaattcccctacatcaaccccactttatcatacttctcttagcttataatttattttttttataaaacctttgttcttctcctccacaaatattcatatatattaacttttttttttaaaaaaaaatggttggtaaaaataattccaatcacctccctcctggatttaggtttcatccaacagatgaggaattaatcatgtattatcttcgaaatcaagctacctccaagccttgtccatcttcaatcatccccgaagttgatgtctataagttcgatccctgggaattgcctgaaaaaactgaatttggagaaaaagaatggtatttcttcaccccacgtgatagaaaatatccaaatggtgttaggccaaatagggcagctgtgtctggttattggaaagccacaggcacagataaaggtatatatagtggaacaaaatatgttggtattaaaaaagctcttgtattttataaaggaaaaccccctaaaggtatcaagactgattggatcatgcatgaatatagattgagtgaatcaaggactcaaccaactaggccaaatggatccatgaggttggatgattgggtactttgtagaatttataagaagaagaatttggaaagagctatagagatgatgaaagttgaagaagacacacaagaacctcaaataatgagtgttactaatcctattcatgaaagtgtggcttctaatggacaacaaacattgaaactaccaaggacttgttcactatctcatttattagaaatggattattttggatcaatttcacaactatttgatgacaataattcatacaatactattagccaaaataatacattaatgaccaatgttaatggatatgttatgcctcatcaagccatggagaaatttcaactaggggaagtgtcacaaatttctatgaatccatcctatcaatttcagtaagaaggcggtacgcggcgattaaatatttcgacaacaataattactatgcctcaatttaaaacaagttgattactagtatctcatcaataattgtaacgtatagatgaaatttaaaggtcatggtactataggt

SEQ ID NO: 102 - protein, 275 aa, Solanum lycopersicum
MVGKNNSNHLPPGFRFHPTDEELIMYYLRNQATSKPCPSSIIPEVDVYKFDPWELPEKTEFGEKEWYFFTPRDRKYPNGVRPNRAAVSGYWKATGTDKGIYSGTKYVGIKKALVFYKGKPPKGIKTDWIMHEYRLSESRTQPTRPNGSMRLDDWVLCRIYKKKNLERAIEMMKVEEDTQEPQIMSVTNPIHESVASNGQQTLKLPRTCSLSHLLEMDYFGSISQLFDDNNSYNTISQNNTLMTNVNGYVMPHQAMEKFQLGEVSQISMNPSYQFQ

SEQ ID NO: 103 - DNA, 1117 bp, Solanum lycopersicum
tttcctccctatagaattaaaaaaaaaaaaactaaaatttcattttctctcaatttccttgcttttgtttaaaaaaaacaaagaaaaagaagaattgttccattttatttatttctatatttttgtataaaatggttggaaaaattagctctgatcttcctcctggatttaggtttcatccaacagatgaggaattaatcatgtactatcttcgatatcaagctacttcaaggccatgccctgtctcaattatccccgaaattgatgtctacaagttcgatccctgggtattacctgaaaaggcagaatttggagacaatgagtggtatttttttacccctagagatagaaaatatccaaatggtgttaggccaaatagagcagctgtgtcaggttattggaaggctactggcactgataaagcaatttatagtgcaaataaatatgttggaattaaaaaagcacttgttttttacaaaggcaaaccaccaaaaggtgtcaagactgattggatcatgcatgaatatcgtttgagtgattcaaagtctcaaacatctaagcaaagtggatctatgaggttagatgattgggtactatgtagaatttataagaagaaaaatttggggagaactatagagatgatgaaagttgaagaagaagaattagaggcacaaaatgtgagtactacaaataatgaaattgaagttgttggtggaccacaaacaatgaaattaccaagaatttgttcattgtcacatctattggaattggattattttggatcaattccacaattactaagtgacaatttattatatgatgaccaaggttacacaatgaataatgtgaataacacaagtaatgtggatcaagtatcatcacaacaacaaaatacaaataatattacaagcaataattgtaatattttcttcaattatcaacaaccactttttgtgaatccaacatttcaatctcagtgatgatcaatagcggagacagaattttcatcaagaatcttctaaatagattctcagttatatcaaaggggttcaatatgtagataattatatataaaatattttttcaacgaagaggttcatggccgggtgaaaatata

SEQ ID NO: 104 - protein, 282 aa, Solanum lycopersicum
MVGKISSDLPPGFRFHPTDEELIMYYLRYQATSRPCPVSIIPEIDVYKFDPWVLPEKAEFGDNEWYFFTPRDRKYPNGVRPNRAAVSGYWKATGTDKAIYSANKYVGIKKALVFYKGKPPKGVKTDWIMHEYRLSDSKSQTSKQSGSMRLDDWVLCRIYKKKNLGRTIEMMKVEEEELEAQNVSTTNNEIEVVGGPQTMKLPRICSLSHLLELDYFGSIPQLLSDNLLYDDQGYTMNNVNNTSNVDQVSSQQQNTNNITSNNCNIFFNYQQPLFVNPTFQSQ

SEQ ID NO: 105 - DNA, 1563 bp, Solanum lycopersicum
cacccttttccctcttttatacttctctctgttcattttatatgtatctgttgtttctctggtgggtttcttgaaaggaatttttctctgtttttttttggttccagaaatggagcaagagggtgcacttgttttagcaccggcaccggtggggccggtgcagccggtgccgccagtggcgccgagaagggcagtgggagttttttcggcgacgtctttagcaccgggattcaggtttcatccaactgatgaggaattggtgaggtactatttgaggaggaaggcttgtggaaaaccatttcgagttgaggctgttgctgaaattgatgtttacaaatctgagccttgggaccttaaagattattcagctctgaagaccagagatttagagtggtattttttcagccctgtagataggaagtatgggaatggatctagacttaatagagccactggagagggctactggaaagctactggaaaggatcgtaatgtgaaccacaattctaaaatcattggaatgaagaaaacgcttgttttccataagggtcgagctcctgatggagagagaactaactgggtaatgcatgagtacagacttgacgataaagaattggatgcagctggagttgtgcaggatgcctttgtactctgtcggatcttccagaaaagtggattagggcctcctaatggcgacagatacgcaccatttattgaggaggaatgggatgacgacacagcccctctggttgccggaggagtgacagagaatgacgtggccaatggtaatcaagcacgagtcgagggaaatgagtttgaccaggatgtgcgtggtaaggctccttgcgtaagtgagaacctgtttgagcctcgagctctttcgtttgtttgcaagagggaaaggtcagaggatctcgaactactctctttatctcaaagcaaaagatccaagcatgatgatcctagctctagccatgcaaatggttctgaagattcaaccactagccaacaggatccaccaacaaagacgatggcgacaacttactcccctactctcttaacatttcccttgttagatgaaccaaaagaagctccacctactaactcctttaattttgactcttccaatcttgaaaaatctgttcctccaggttacttgaagttcattagcaatttagagaaccagatactaaacgtctccatggaaagggagactgcgaagattgaagtgatgagagctcaagctatgatcaacattcttcaatcacgtctcgatttcttgaacaaggagaacgaggacttgaggggactcattcgtcgaggttagatggtaacaacaactccaaagtaatgctagttatgcagcaactgttggccgggcagagggctccgttgtatcggagtcctcaattagtagaactgtcttttaagttagaagaaactgaaaatggctagctatgtaataagtttatttacctagttttgctataaacttgtacctatgatgctatcgacataatgtcgtatgtttaactagttataaataaagtgccttcagttgcc

SEQ ID NO: 106 - protein, 405 aa, Solanum lycopersicum
MEQEGALVLAPAPVGPVQPVPPVAPRRAVGVFSATSLAPGFRFHPTDEELVRYYLRRKACGKPFRVEAVAEIDVYKSEPWDLKDYSALKTRDLEWYFFSPVDRKYGNGSRLNRATGEGYWKATGKDRNVNHNSKIIGMKKTLVFHKGRAPDGERTNWVMHEYRLDDKELDAAGVVQDAFVLCRIFQKSGLGPPNGDRYAPFIEEEWDDDTAPLVAGGVTENDVANGNQARVEGNEFDQDVRGKAPCVSENLFEPRALSFVCKRERSEDLELLSLSQSKRSKHDDPSSSHANGSEDSTTSQQDPPTKTMATTYSPTLLTFPLLDEPKEAPPTNSFNFDSSNLEKSVPPGYLKFISNLENQILNVSMERETAKIEVMRAQAMINILQSRLDFLNKENEDLRGLIRRG

SEQ ID NO: 107 - DNA, 740 bp, Solanum lycopersicum
aattacaacacctcttcatcttctctctctaactcctaaatcttaccaaaattctgaatttttcgttgctgtatcaactttaggcggttatactgaagaatttcttgttaaattgttagttttttcacaaaatttggttcaaaattgtccatttttgttaactttgactgatggaatcaaaagggtcggcgaaatggatggcgccggggttcagatttcatccgacggatgaggagttggtcaggtactatctccggcggaaggttattggtaaacctttgagatttgatgctatttctgagatcgatatttataaagttgaaccatgggatcttccgggtatgtcgaggttgaaaacaagagatttggaatggtacttcttcagtgtgcttgataggaagcatggtaatggagcaaaaaccaatcgggcgacagaacgaggctactggaagacaactggaaaggacagagctgtccatcataagtctaaggttgtcggtatgaagaagactctagtttttcatagtggtcgggccccaaaaggtgagaggactaattgggtcatgcatgagtacaaactaattgatgaagagttgcaaaaagctggaattgttcaggatgcacttgtgttatgtagagtcttccagaagagtggcgcagggccaaaagatgggtgaaaagtatggggctccttttgttgaggaggaaatggaggatgatgagttagaaattgttcccaa

SEQ ID NO: 108 - protein, 168 aa, Solanum lycopersicum
MESKGSAKWMAPGFRFHPTDEELVRYYLRRKVIGKPLRFDAISEIDIYKVEPWDLPGMSRLKTRDLEWYFFSVLDRKHGNGAKTNRATERGYWKTTGKDRAVHHKSKVVGMKKTLVFHSGRAPKGERTNWVMHEYKLIDEELQKAGIVQDALVLCRVFQKSGAGPKDG

SEQ ID NO: 109 - DNA, 1363 bp, Solanum lycopersicum
aaaatttcaaaaaaaaatctatatttttcttctctctctacattttgcttacttgtgagaaggaaaaaaaatgggtgttcaggaaatggatcctcttacacagctaagcttaccacccgggttccggttttacccgactgatgaagaacttttagttcaatatttatgccgtaaagttgctggtcatgatttttctctgcaaattattgctgaaattgatttgtacaaattcgatccatgggttcttccaagtaaggcgattttcggagaaaaagaatggtatttcttcagtccaagagatcggaagtatccgaatggatctagaccaaacagagtagctgggtctggttattggaaagcaactggaactgataaagttattactacagacggtagaaaagtcggaatcaaaaaggctttagtgttttacattggtaaagcacctaaaggaactaaaacaaattggattatgcacgaatacaggctcagtgaacctacaacgaaaactggaagttcaaggctcgacgattgggttctatgtaggatttacaagaagaattcaggtggacaaaaatcgagttgctctgatttacagaacaaggatataagtcatgcttcatcatcgtcatcgtcatctcagtttgatgatatgctggaatctctaccggcaattgaagatcgttatttctcattgccgagggtgaattctataaggaattttcaacaaaatgacaagatcaatcttcaacaattgagctctgggaacttcgattgggctgctatggcgggattaaactcattcccggaattacgtaccggaaatcaagttccaacgccgggaaatcaaactccggtgctgataaacaccaatcagtatcacaatcacatcgacaatttgaataatttcaacgaatttttcgccaattcaacggcgttaaattttcacggtgaagttaagtttgaaggaggagttgatcaagaagtagaaagcagtgttagagctcaacgacttatcagtgttaacccgggtttcttccaagagaactcaaccgggttttcaagttcttatacaaactcggtacgcgatccatttgggattcggtacccgacccaaacagtaaatatgggttttactgggtaaatagtggaagggtaaaatgtgaatgctcataaattaacttgaagacaaatttggaaaaattgtcatggttatttaggttgaaagggcattttattctttggttggggattaacatagcaaaaaaaaaaaaacaagattttgttcatgcaattatgcacatatacaatgtattttgttgtactaaaatttatagctttggtaatgtacttattataggaatgc

SEQ ID NO: 110 - protein, 356 aa, Solanum lycopersicum
MGVQEMDPLTQLSLPPGFRFYPTDEELLVQYLCRKVAGHDFSLQIIAEIDLYKFDPWVLPSKAIFGEKEWYFFSPRDRKYPNGSRPNRVAGSGYWKATGTDKVITTDGRKVGIKKALVFYIGKAPKGTKTNWIMHEYRLSEPTTKTGSSRLDDWVLCRIYKKNSGGQKSSCSDLQNKDISHASSSSSSSQFDDMLESLPAIEDRYFSLPRVNSIRNFQQNDKINLQQLSSGNFDWAAMAGLNSFPELRTGNQVPTPGNQTPVLINTNQYHNHIDNLNNFNEFFANSTALNFHGEVKFEGGVDQEVESSVRAQRLISVNPGFFQENSTGFSSSYTNSVRDPFGIRYPTQTVNMGFTG

SEQ ID NO: 111 - DNA, 1189 bp, Triticum aestivum
  misc_feature (992)..(992): n is a, c, g, or t
cgttacgttcctgccgctgccgattgagtccatatcatcatcaccagttccaaatgagggaaaaacaagtgatggagcacggcgagcaggagcagcacgccatggacctgccgccgggcttccgcttccaccccaccgacgaggagctcatcacgcactacctggccaagaaggtcgccgacgcccgcttcgccgccctcgccgtctccgtcgccgacctcaacaagtgcgagccctgggacctgccatcgctggctaggatgggggagaaggagtggtacttcttctgtctcaaggaccgcaagtacccgacggggctcaggaccaacagggccaccgagtccggctactggaaggccaccggcaaggacaaggacatcttcaggggcaaggccctcgtcggcatgaagaagacgctcgtcttctacaccgggagggcgcccaagggcgacaagtccggctgggtcatgcacgagtaccgcatcaacgccaagctccacgccgccagcaccagccgcggctccctctccggcggcagggccgcatcgtccaggaacgagtgggtgctgtgcagggtgttcaagaagagcctgctaggcggcgtagtgtcgtcggcgccggcgtcgtcagcagcaaagaaaagcggcgtggggacgatggaggagatagggtcctccgtggccgccgtcaccccgctccctccgctcctggacatgtccggctccggcgcctccttcgtcgacccggcggcgcacgtgacctgcttctcccacacgcgctggaggcgggccagttcttcaaccgacggcgacgggcggctgcgccacggacacggaccacacgggctagcctcatcgtactccccgttgggcagcttcacgcagtacggggggcagctgcaccacggcgtgagcctgagcctggtgcagctcctgaagagcagcgggtaccacgcggtctcgccgacgacatgcgccgggcggcaatagcancaccagcgggggnttgtagggcnaccgggaaagctaaccgtcccaggaaccggcctcactcgagctgacccgaaatttttcctcttggncaaaattgaaccgagcgggccgggggttatgacaaaaacctcccatcccggttcggggaaagaccaccattttggctttccgggttgactggtaatcccgagggcgaa

SEQ ID NO: 112 - protein, 311 aa, Triticum aestivum
MREKQVMEHGEQEQHAMDLPPGFRFHPTDEELITHYLAKKVADARFAALAVSVADLNKCEPWDLPSLARMGEKEWYFFCLKDRKYPTGLRTNRATESGYWKATGKDKDIFRGKALVGMKKTLVFYTGRAPKGDKSGWVMHEYRINAKLHAASTSRGSLSGGRAASSRNEWVLCRVFKKSLLGGVVSSAPASSAAKKSGVGTMEEIGSSVAAVTPLPPLLDMSGSGASFVDPAAHVTCFSHTRWRRASSSTDGDGRLRHGHGPHGLASSYSPLGSFTQYGGQLHHGVSLSLVQLLKSSGYHAVSPTTCAGRQ

SEQ ID NO: 113 - DNA, 1071 bp, Triticum aestivum
  misc_feature (625)..(625): n is a, c, g, or t
ggttgtacttcgacccacgcgtccggacgaaatcacctctaacatctcgatctacctcttcctcctcctcagctctcgttccatcaggttcttccacagcgtagcaaggcaatctagtagatcctccatgtcggacgtgacggcggtgatggatctggaggtggaggagccgcagctggcgcttccaccggggttccggttccaccccaccgacgaggaggtggtcacccactacctcacccgcaaggtcctccgcgaatccttctcctgccaagtgatcaccgacgtcgacctcaacaagaacgagccgtgggagctcccgggcctcgcgaagatgggcgagaaggagtggttcttcttcgtgcacaagggtcggaagtacccgacggggacgcgcaccaaccgggcgacgaagaaggggtactggaaggcgacggggaaggacaaggagatcttccgcggcaagggccgggacgccgtcctcgtcggcatgaagaagacgctcgtcttctacaccggccgcgcccccagcggcgggaagacgccgtgggtgatgcacgagtaccgcctcgagggcgagctgccccatcgccttccccgcaccgccaaggacgattgggctgtntgccgggtgttcaacaaagacttggcggcgaggaatgcgccccagatggcggcggcggccgacggtggcatggaggacccgctcgcctccctcgatgacttgctcatcgacaccgacctgttcgacgacgcggaccttccgatgctcatggaatccccgtctggcggtgacaacttcggcggggctttgaagctccaccttgaagggcggcccctcccgtttgagcccggaagcggagcctccgggtggctgcatccgcaggagcaagcaatgaccccaaattacttttttattcccggggaacgggcaacggtgaatttttggggggggccgggggaactcccccctacccggggttatgggggtgcccaagagggccgcgaaacttaaaannttttnnnncccccgccncggggcgggttnccttctttcctctggggccgggt

SEQ ID NO: 114 - protein, 295 aa, Triticum aestivum
MSDVTAVMDLEVEEPQLALPPGFRFHPTDEEVVTHYLTRKVLRESFSCQVITDVDLNKNEPWELPGLAKMGEKEWFFFVHKGRKYPTGTRTNRATKKGYWKATGKDKEIFRGKGRDAVLVGMKKTLVFYTGRAPSGGKTPWVMHEYRLEGELPHRLPRTAKDDWAVCRVFNKDLAARNAPQMAAAADGGMEDPLASLDDLLIDTDLFDDADLPMLMESPSGGDNFGGALKLHLEGRPLPFEPGSGASGWLHPQEQAMTPNYFFIPGERATVNFWGGPGELPPTRGYGGAQEGRET

SEQ ID NO: 115 - DNA, 1421 bp, Triticum aestivum
gcagcatttcatatgcagtagccatcttctcctcctcctcccgctttccagctagccacctagctcactatcacatcatccagcagcccacaccaactcatattgattccgttcccacctgctcgccatcgccagccatgccaatgggcagcagcgccgccatgcccgccctccctcccggcttccggttccaccccaccgacgaggagctcatcgtccactacctcggcaggcaggccgcgtccatgcccagccccgtgcccatcatcgccgaggtcaacatctacaagtgcaacccatgggacctccccggcaaggccttgttcggggagaatgagtggtacttcttcagcccccgggatcgcaagtaccccaacggcgcgcgccccaaccgcgccgccgggtccggctactggaaggccaccggcaccgacaaggccatcctgtccacgccggccaacgagagcatcggcgtcaagaaggcgctcgtgttctacaggggcaagccgcccaagggcgtcaagaccgactggatcatgcacgagtaccgcctcaccgcagccgacaaccggaccaccaagcgcagaggatcctccatgaggctggatgactgggtgctgtgtaggatccacaagaagtgcggcaacttgcccaacttctcctcctctgaccaggaacaagagcatgagcaggagagctccaccgtggaggactcgcagaacaaccacaccgtgtcgtcgcccaagtccgaggccttcgacggcgacggcgacgaccacctccagttgcagcagttccgccccatggcgatcgccaagtcgtgctccctcaccgacctgctcaacaccgtcgactacgccgcgctctcgcacctcctcctcgacggcgccggcgcctcgtcgtcggacgccggagcagactaccagctgccgccggaaaacccactcatctactcgcagcctccatggcaacaaacgctacactataataacaacaacggctacgtgaacaacgagaccatcgacgtgcctcagctacccgaggcgcgcgtagatgactacggcatgaatggcgataagtataacggcatgaagaggaagagatccagcggcagcttgtactgcagccagctgcagctcccagcggatcagtacagcggcatgctgatccatccgttcctcagccagcagctgcacatgtgaagaaccagagagcttggatcgaagagatcatcattgttccatttgaacttgctgtagatcgagaggatcccccgctgtggcagataggcagggatctagctagcttgctgtgtcgtgaacgaccgaccgataagaacggacaaatttcagaattcttggtgtagcacaaagctgtaaattacgggggtttattgtgaaataaattaatccagtattatc

SEQ ID NO: 116 - protein, 354 aa, Triticum aestivum
MPMGSSAAMPALPPGFRFHPTDEELIVHYLGRQAASMPSPVPIIAEVNIYKCNPWDLPGKALFGENEWYFFSPRDRKYPNGARPNRAAGSGYWKATGTDKAILSTPANESIGVKKALVFYRGKPPKGVKTDWIMHEYRLTAADNRTTKRRGSSMRLDDWVLCRIHKKCGNLPNFSSSDQEQEHEQESSTVEDSQNNHTVSSPKSEAFDGDGDDHLQLQQFRPMAIAKSCSLTDLLNTVDYAALSHLLLDGAGASSSDAGADYQLPPENPLIYSQPPWQQTLHYNNNNGYVNNETIDVPQLPEARVDDYGMNGDKYNGMKRKRSSGSLYCSQLQLPADQYSGMLIHPFLSQQLHM

SEQ ID NO: 117 - DNA, 1244 bp, Triticum aestivum
  misc_feature (1243)..(1243): n is a, c, g, or t
cccacgcgtccggctaaaacccgcctctcgctccctctttcacacacgcctcttcctcttcctctctctctcggcttccatttcctcttcttctagctagcttgcttcggtgcccggcggagttggggcacgcgcgcgcgcgcggagatgtctgaggcgtcggtggtaaaccaggcggaggtggaggacgcggcggcggcggcggcggccgggctggacctgccgccggggttccggttccaccccacggacgaggagatcatctcgcactacctcacccccaaggcgctcgaccaccgcttctgctccggcgtcatcggcgaggtcgacctcaacaagtgcgagccatggcatctcccaggcaaggccaagatgggcgagaaggagtggtacttcttttgccacaaagaccgcaagtacccgacggggacgaggacgaaccgcgccaccgaaagcggctactggaaggccaccggcaaggacaaggagatcttccgggggaggggcatcctcgtcggaatgaagaagacgctcgtcttctacctaggccgcgccccccgcggcgagaagaccggctgggtcatgcacgagttccgcctcgagggcaagctcactcacccgctcccgcgctccgccaaggacgagtgggccgtgtccaaggtgttcaacaaagagctcacggccaccaacggggcaatggcagcggcggaggccgggatcgagcgagtcagctccttgggcttcatcactgacttccttgactctggggagctgccgcccctcatggaccctcccttgggcggcgacgtcgacgaagtcatcgatttcaagtccacctctgcctacgccaccggtgcccattccggactccaggttaagatggaacagcacatgccgccgcacatgatgtactcgagcccctacttctccctgccggccgccaactccggcgacatgtcgccggcgatccggaggtactgcaaggcggagcaggtctcggggcagacgtctgcgctcagcccgtcccgcgagaccgggctgagcaccgaccccaacgccgccggttgcgcggagatctcatcggcggcgacaccgtcgtctcagaatcaagagttccttgaccaattcgacgagtaccccgccctgaacctcgccgacatttggaatactgaaaccggccggagctgcacctaccacatgattctcgctcgattggttctggctcgttgattaattactcaant

SEQ ID NO: 118 - protein, 360 aa, Triticum aestivum
MSEASVVNQAEVEDAAAAAAAGLDLPPGFRFHPTDEEIISHYLTPKALDHRFCSGVIGEVDLNKCEPWHLPGKAKMGEKEWYFFCHKDRKYPTGTRTNRATESGYWKATGKDKEIFRGRGILVGMKKTLVFYLGRAPRGEKTGWVMHEFRLEGKLTHPLPRSAKDEWAVSKVFNKELTATNGAMAAAEAGIERVSSLGFITDFLDSGELPPLMDPPLGGDVDEVIDFKSTSAYATGAHSGLQVKMEQHMPPHMMYSSPYFSLPAANSGDMSPAIRRYCKAEQVSGQTSALSPSRETGLSTDPNAAGCAEISSAATPSSQNQEFLDQFDEYPALNLADIWNTETGRSCTYHMILARLVLAR

SEQ ID NO: 119 - DNA, 1034 bp, Triticum aestivum
ccacgcgtccgaccggcaactacttcattcccctcgactcgacaccagcgtcttccctcccatcagttgttgctagctttgtaggctgtagctaggtggatcatttcactccagctacaacactgtcttctttatctatcctctagtgatcgctgtgtgtgagactcacccatcgaccatgagcatctccgtgaacgggcagtcggtggtgccgccggggttccggttccacccgacggaggaggagctcctcacctactacctcgtcaagaaggtggactcacagcgcatcgccctcggcgtcatccccgacatcgacctcaacaagctcgagccatgggacatccaagcgcggtgccggatcggaactggcccgcagaacgactggtacctgtgcagccacaaggacaagaagtaccccacggggacgcgcaccaaccgtgccaccgacgccgggttctggaaggctaccggccgggacaagcccatctactccgccgccggctccggccgcatcggcatgcgcaagacgctcgtcttctacaagggccgtgccccgcgtggccacaagtccgactggatcatgcacgagtaccgcctcgacgacgccgtccccgtcaaccccacggccggcaacgcctcctactactccagtgactcattcccggtacatgctctactactacagtctctccgcaatactgtagtacgtcgcaattgcattcacggcggccggccggccggacgacagggatggagatagctccctagtgggtgatccccgtgaaaaatgtttatatggggcgcgctttttcttttctgggtatttaaggcgtgggcaattatccaaatgtaaaaaaacaggggtttaataaaaaattgtggtccgggtggtgtgttgggaattgggaacccccctgtaatttgccccagggggctaaattgggaaagtgggagccggtgttccctttccctttcgaaggggcccgccctcacaaaaaagggggggtaaggggttaataaggtcggggtcc

SEQ ID NO: 120 - protein, 194 aa, Triticum aestivum
MSISVNGQSVVPPGFRFHPTEEELLTYYLVKKVDSQRIALGVIPDIDLNKLEPWDIQARCRIGTGPQNDWYLCSHKDKKYPTGTRTNRATDAGFWKATGRDKPIYSAAGSGRIGMRKTLVFYKGRAPRGHKSDWIMHEYRLDDAVPVNPTAGNASYYSSDSFPVHALLLQSLRNTVVRRNCIHGGRPAGRQGWR

SEQ ID NO: 121 - DNA, 1380 bp, Triticum aestivum
ttaccatcacccctgaatccattgcacataaagcctccaccatggcagaccaccttcaagttcagcagaaacagttggtactaccgccggggtttaggttccacccgacggatgaggagatcatcaaattctatgtggtccccaaggtgctcgatgaagccttcgttgtcgcggcgattgaggatgtaaacctcaacaagtacgagccatgggagctaccagagaaggcgaagatgggggagaaagagtggtacttttactcccgaaaggatcgcaagtaccccactgggatacgaacgaaccgggcgacggaggccggctattggaaggccaccggaaaggacaaggagatcttccagccacctttcacactcatcggcatgaagaagacacttgtcttctacaagggccgggcgcctaggggggagaagaccaattggatcatgcatgagtataggctcgagagcaacaagaagctgacatccaacccatccatcaccacccgcaccgtcaccagaaccaacacggcttccaaggaacagtgggtggtttgcaggatcttccataagagcaccggactaaagaagatggtgacaccgtcatataacatgccaatgtacacaggagcagaacatcagcaggcctttgtcgacttagatacattgcctcctctcatggaatatgacatgtcgtcaacatgtgcgcatgcaccgctgtttcctggagcttcttcatacaagtcgcacgatgtcggaactggctcatcgatgatgggcagtgtggcgcttcctatgatgaattaccactacatcgggaacaaccaccaccaccaaatgatggccaacccaacactgcctctgtcgctctaccaacaccaacaacaacatcaacagatgatgatgcatatgggtgcagatcagggtctcatggtcggggctcaacctggaagtgggttggcgtcgatgctgtcacaggaggacactgtggccgggctgaggaacaactacccagggaacaccgctacaacagcagttggcgagacctcattgatgaacatgggtatggatgacatttggcagaactgatgatcagataacccaagacgacatatatatgtgtgtgcatgcataaattaataatccatcagacggtcagttgatatatgccaacccgtatattggttggtgccgcatttttatttgttagtgtgaccttgagagagtttgcaatcatggcatggcttggcatgccactcttgtgctagtttttcaattgtgtgtatgtgtattggcttgtatatgcactacccaccagggtatacctagctacatatgcatgtgttgtaaatgtgtgtacatcatgcatctttgaaacccaaaa

SEQ ID NO: 122 - protein, 347 aa, Triticum aestivum
MADHLQVQQKQLVLPPGFRFHPTDEEIIKFYVVPKVLDEAFVVAAIEDVNLNKYEPWELPEKAKMGEKEWYFYSRKDRKYPTGIRTNRATEAGYWKATGKDKEIFQPPFTLIGMKKTLVFYKGRAPRGEKTNWIMHEYRLESNKKLTSNPSITTRTVTRTNTASKEQWVVCRIFHKSTGLKKMVTPSYNMPMYTGAEHQQAFVDLDTLPPLMEYDMSSTCAHAPLFPGASSYKSHDVGTGSSMMGSVALPMMNYHYIGNNHHHQMMANPTLPLSLYQHQQQHQQMMMHMGADQGLMVGAQPGSGLASMLSQEDTVAGLRNNYPGNTATTAVGETSLMNMGMDDIWQN

SEQ ID NO: 123 - DNA, 1241 bp, Triticum aestivum
ccgcaccacagaatcatatactatggaaactctacgtgacatggctctgccaccaggatttggattccatcccaaggacactgaactcgtttctcactatctgaaaaagaaaatactcggtcagaaaattgaatatgacattatacctgaggtggatatatacaagcatgaaccatgggatttacctgcaaagtgcaatgttccaacccaggataacaagtggcatttctttgctgctcgagacagaaagtatcctaatggtgctcggtcaaatagggcaacggttgctgggtattggaagtctactggaaaagaccgggccattaaggtggataagcggaccataggaacaaagaaaactttagtttttcatgaaggtcggcctcccactggaaaacgtactgagtggattatgcatgaatactacatagatgagaacgaatgtcaagcctgccctgatatgaaggatgcctttgttctatgtaaagttactaaaagaattgactggacatcagagaatggtaacgaggtgggcaataataaccctcagccacaacaagcaaatgttgctgctattttagctgtcagtgttgaacaaccagatactgctgcatcatcaattattggtgatgaacttccaagtgatgctgctaccgtagctatccctgctcacacaactccagatgggaatgatgaccttaaagaatggctggaagaactgcttgatacctcctttgatccttctgctaacactgcagtggattctatttctgctcaactgtctccggatgaacaaaatgctgaatcatcgaacattggtgctatggctccgaaggtggaactggactatgccagtcctaaccagactgtggtagacgatacagacttcctgttgccagatgatattcactccatgttgtatcctggtagtgatgacttcacttcatggcagcaaacatactttacggcggcggatcccttcagtctgtcaaacaccttcatggatgaatttcagatgaaagagctacagttacctctggaaaataatgggcctaatctgtttgaccctgctgatactggatttgtgtgcgaacccgtatgggggaacatctggagctagggttcaacattaaaggctcgatttcagaaaattttgggagatggttactacagctctaatccataaaccaaatatcagttcgttgtcataacggccatctggatccatgacaaagggaacc

SEQ ID NO: 124 - protein, 365 aa, Triticum aestivum
METLRDMALPPGFGFHPKDTELVSHYLKKKILGQKIEYDIIPEVDIYKHEPWDLPAKCNVPTQDNKWHFFAARDRKYPNGARSNRATVAGYWKSTGKDRAIKVDKRTIGTKKTLVFHEGRPPTGKRTEWIMHEYYIDENECQACPDMKDAFVLCKVTKRIDWTSENGNEVGNNNPQPQQANVAAILAVSVEQPDTAASSIIGDELPSDAATVAIPAHTTPDGNDDLKEWLEELLDTSFDPSANTAVDSISAQLSPDEQNAESSNIGAMAPKVELDYASPNQTVVDDTDFLLPDDIHSMLYPGSDDFTSWQQTYFTAADPFSLSNTFMDEFQMKELQLPLENNGPNLFDPADTGFVCEPVWGNIWS

SEQ ID NO: 125 - DNA, 1290 bp, Triticum aestivum
tctatccttcgttgctgtggggagcaaccaggcggccgggcacaaaaggcagcaggagatctccaagatcgatcttagggccggcgagggagatcgagagccagcatgcatccgagtggcggcgcgctgtcagtgccaccgggcttccggttccatccgacggacgaggagctcctctactactacctgaggaagaaggtcgcctatgaggccatagatcttgacgtcatcagggagatcgacctcaacaagctcgagccatgggacctcaaagatcggtgcaggataggcacgggcccgcagaacgagtggtacttcttcagccacaaggacaagaagtacccgacggggacgaggacgaaccgggccaccacggcggggttctggaaagccacgggccgcgacaaggccatcttcctcggcagcggcgccaggaggatcggcatgaggaagacgctggtgttctacgtcgggagggctccccacggcaagaagaccgactggatcatgcacgagtaccgcctagacgaagacaacgtcgaggtccaggaagatggctgggtggtgtgtagggttttcaccaagaagagctacacgagaggcgtgaacccggcggagatggcggggtacgacgacgacgagctcctccaccctttcccggttccatcttccaccgccggcgcaatgcaaatgatcacggaccagaagcacatcatgcacaaccccggccacctcatgcagcagcagcagcagcactacgacttcccttccttcgacgcgtccatgcagctcccgcagctcatgagcgccgagcacgagcagcagacgctctcctccttcctccccggcgcaccagccgccgtcgccatgaggctcgcacgagctcgagtgctcacagaacctgatgaagctcacgtcgagcggcaccaacggaatgctccaccatcacggtggcggtggcggtggaagcgacccccgcttccccggcacggattggtccatcctggacaagctcctcgcctcgcaccagaacctggaccagctcttccagggcaaggttgccgccgcagcagcagcatcaatggcaccttaccagcagcagcatcagcagcaactcatggagcaacttggaggcagcagcacctcatccatgcagaggatgccactgcagtacctcggctgtgaggccgccgacctcctcaggttctccaagtacttaaatggagctttgcctagctagctgccatgctatatagtactgttgatctgatcagctcctgtttttcatgt

SEQ ID NO: 126 - protein, 359 aa, Triticum aestivum
MHPSGGALSVPPGFRFHPTDEELLYYYLRKKVAYEAIDLDVIREIDLNKLEPWDLKDRCRIGTGPQNEWYFFSHKDKKYPTGTRTNRATTAGFWKATGRDKAIFLGSGARRIGMRKTLVFYVGRAPHGKKTDWIMHEYRLDEDNVEVQEDGWVVCRVFTKKSYTRGVNPAEMAGYDDDELLHPFPVPSSTAGAMQMITDQKHIMHNPGHLMQQQQQHYDFPSFDASMQLPQLMSAEHEQQTLSSFLPGAPAAVAMRLARARVLTEPDEAHVERHQRNAPPSRWRWRWKRPPLPRHGLVHPGQAPRLAPEPGPALPGQGCRRSSSINGTLPAAASAATHGATWRQQHLIHAEDATAVPRL

SEQ ID NO: 127 - DNA, 1665 bp, Triticum aestivum
  misc_feature (23)..(23): n is a, c, g, or t
tcgacccacgcttccgcttgttnctaaaacccgcctctctctccctctttcacacccgcctcttcctcttcctctctctctcggcttccatttcctcttcttctagctagcttgcttcggtgcccggcggagttggggcacgcgcgcgcgcgcggagatgtctgaggcgtcggtggtaaaccaggcggaggtggaggacgcggcggcggcggcggcggccgggctggacctgccgccggggttccggttccaccccacggacgaggagatcatctcgcactacctcacccccaaggcgctcgaccaccgcttctgctccggcgtcatcggcgaggtcgacctcaacaagtgcgagccatggcatctcccaggcaaggccaagatgggcgagaaggagtggtacttcttttgccacaaagaccgcaagtacccgacggggacgaggacgaaccgcgccaccgaaagcggctactggaaggccaccggcaaggacaaggagatcttccgggggaggggcatcctcgtcggaatgaagaagacgctcgtcttctacctaggccgcgccccccgcggcgagaagaccggctgggtcatgcacgagttccgcctcgagggcaagctcactcacccgctcccgcgctccgccaaggacgagtgggccgtgtccaaggtgttcaacaaagagctcacggccaccaacggggcaatggcagcggcggaggccgggatcgagcgagtcagctccttgggcttcatcactgacttccttgactctggggagctgccgcccctcatggaccctcccttgggcggcgacgtcgacgaagtcatcgatttcaagtccacctctgcctacgccaccggtgcccattccggactccaggttaagatggaacagcacatgccgccgcacatgatgtactcgagcccctacttctccctgccggccgccaactccggcgacatgtcgccggcgatccggaggtactgcaaggcggagcaggtctcggggcaacgtctgcgctcagcccgtcccgcgagaccgggctgagcaccgaccccaacgccgccggttgcgcggagatctcatcggcggcgacaccgtcgtctcagaatcaagagttccttgaccaattcgacgagtaccccgccctgaacctcgccgacatttggaagtactgaagccggccggagctgcacctaccacatgattctcgctcgattggttctggctcgttgattaattactcagttggattacttaacttggtgtgtgatgctcgtgttagtggtttgctgatgcagcatcatcttctttccggaggactctcgggtcggatcaggttactatctatccagacagaccgacagacctccagggattagctaggcatccttaatcatgttattaaattaattagtcctaggttcatatcttgatctgtgtgtacatatcactacaactatattctgattgtgattaattactagcagtagcagcatgatggttatacaattaatgtgtgtagattaggggggacgacttcagatccatttgtgaatcgattaatttgggcgccgttgatgagccagccagctagctctactactagtagctttgtatgtacatgtaccaccattgatcattaattgcttgagtttgac

SEQ ID NO: 128 - protein, 297 aa, Triticum aestivum
MSEASVVNQAEVEDAAAAAAAGLDLPPGFRFHPTDEEIISHYLTPKALDHRFCSGVIGEVDLNKCEPWHLPGKAKMGEKEWYFFCHKDRKYPTGTRTNRATESGYWKATGKDKEIFRGRGILVGMKKTLVFYLGRAPRGEKTGWVMHEFRLEGKLTHPLPRSAKDEWAVSKVFNKELTATNGAMAAAEAGIERVSSLGFITDFLDSGELPPLMDPPLGGDVDEVIDFKSTSAYATGAHSGLQVKMEQHMPPHMMYSSPYFSLPAANSGDMSPAIRRYCKAEQVSGQRLRSARPARPG

SEQ ID NO: 129 - DNA, 1303 bp, Triticum aestivum
  misc_feature (2)..(2): n is a, c, g, or t
tngcttgcttggcctgtccgtttctctctctctctactgcgtgttcctctctcatcgcatcaacaaggagagcaagagatagagagagatgtcgatgagcttcctgagcatggtggagacggagctgccgccggggttccggttccacccgagagacgacgagctcatctgcgactacctcgcgcccaaggtcaccggcaaggtcggcttctccggccgccgcccgcccatggtcgacgtcgacctcaacaaggtcgagccatgggacctccccgtgactgcatcggtgggtggcaaggagtggtatttctatagcctaaaggatcggaaatatgcgacggggcagcgcacgaaccgagctactgtgtcggggtactggaaggcaaccgggaaggatagggtggtggcacgacgtggtgcgctagtggggatgaggaagacattggtgttctaccaagggagggcccctaaggggaggaagacggagtgggtgatgcacgaatacaggttggagggtgcgcatgagcaagcttccaagcaggaggactgggtcctgtgcagagtcatctgcaagaagaaatcaggagttggtgccacccccaggccaaggaacctcagcaacattgtccatggcacacccacagacacctcctcaccaccactgccacctctcatggacaccaccctagcacagctccaggccaccatgaacacctccgccgccgctgccgccgccgcacttgagcaagtgccctgcttctccagcttcagtaataacagtgccagcaacagcagctacctccccatggtgacaggcaacagtaatggcatgggctacatggaccatggcctgcctgactttgggagctacctagaccccgccatgaactgtgacaagaaggtccttaaggcagtgctgagccagcttagctccatgggtggtgaggtggtgccgagcatgtctcctcagatggctgctgcagtgagctctacttggaaccacttctaggcaaatttgtttatttatgcagacaacataagcagacaactggctagctagtagttaatctgcagtgtgtgtgtgtgtgtgtgtgtgtgtgtgtgtgtgtgtgatgatttcatatcaaaatgtataattatgtctgtttggtgcaatgtttcttgagtgtgagtattagtagaggacgcgatgattttttttctccagtcgattttgaaggactaaaaccctggaatgattttatttttgaagaattctctaatgattgttgaactatgtacattcataagtacagggttgcag

SEQ ID NO: 130 - protein, 306 aa, Triticum aestivum
MSMSFLSMVETELPPGFRFHPRDDELICDYLAPKVTGKVGFSGRRPPMVDVDLNKVEPWDLPVTASVGGKEWYFYSLKDRKYATGQRTNRATVSGYWKATGKDRVVARRGALVGMRKTLVFYQGRAPKGRKTEWVMHEYRLEGAHEQASKQEDWVLCRVICKKKSGVGATPRPRNLSNIVHGTPTDTSSPPLPPLMDTTLAQLQATMNTSAAAAAAALEQVPCFSSFSNNSASNSSYLPMVTGNSNGMGYMDHGLPDFGSYLDPAMNCDKKVLKAVLSQLSSMGGEVVPSMSPQMAAAVSSTWNHF

SEQ ID NO: 131 - DNA, 952 bp, Triticum aestivum
  misc_feature (929)..(929): n is a, c, g, or t
acccgcctctctctccctctttcacacacgcctctccctcttcctctcgctcctctctctctctctcggcgtccatttcctcttcttcttgctagtttgcttcaaagttggggcacgcgcgcgcggagatgtctgaggcgtcggttttaaaccaggcggaggtggaggacgcggcggcggcggccgggctggacctgccgccgggcttccggttccaccccacggacgaggagatcatctcgcactacctcacccccaaggcgctcgaccaccgcttctgctccggcgtcatcggcgaggtcgacctcaacaagtgcgagccatggcatctcccaggcaaggcgaagatcggagagaaggagtggtacttcttttgccacaaagaccgcaagtacccgacggggacgaggacgaaccgcgccaccgagagcggctactggaaggccaccggcaaggacaaggagatcttccgggggaggggcgtccttgtcggaatgaagaagacgctcgtcttctacctaggccgcgccccccgcggcgagaagaccggctgggtcatgcacgagttccgcctcgagggcaagctcacccacccgctcccgcgctccgccaaggacgagtgggccgtgtccaaggtgttcaacaaagagctcacggccaccaacggggcaatggcagcggcggaggccgggatcgagcgagtcagctccttcggcttcatcactgacttccttgactctggggagctgccgcccctcatggaccctcccttgggcggcgactcgacgaatcatcgatttcaatccactctgcctacccacggtgccatccggatcaagttaagatggacacacatccgccgcacatgattactcagccctacttcctgcggcgcaatcggcacattcccgggatcggaggtatgaagncgacaagtccggcaacttcctag

SEQ ID NO: 132 - protein, 246 aa, Triticum aestivum
MSEASVLNQAEVEDAAAAAGLDLPPGFRFHPTDEEIISHYLTPKALDHRFCSGVIGEVDLNKCEPWHLPGKAKIGEKEWYFFCHKDRKYPTGTRTNRATESGYWKATGKDKEIFRGRGVLVGMKKTLVFYLGRAPRGEKTGWVMHEFRLEGKLTHPLPRSAKDEWAVSKVFNKELTATNGAMAAAEAGIERVSSFGFITDFLDSGELPPLMDPPLGGDSTNHRFQSTLPTHGAIRIKLRWTHIRRT

SEQ ID NO: 133 - DNA, 1165 bp, Triticum aestivum
agtttgcgtgcttcgatcgtctcgatggctgacttctcgtccagcaacggcgtcccgccggggttccggttccaccctaccgacgaggagctgctgctctactacctcaagaagaagatcggcttcgagaagttcgacctcgaggtcatcagggaggtcgacctcaacaagatcgagccatgggacctccaagagagatgccggatcgggtctgcgccgcagaacgaatggtacttcttcagccacaaggaccgcaagtacccgacggggtcgcggacgaaccgcgcgacgacggccgggttctggaaggcgacggggagggacaagtgcatccgcaccagctaccgcaagatcgggatgcgcaagacgctcgtcttctaccgcggtcgcgccccccacggccagaagtccgactggatcatgcacgagtaccgcctcgaggaaatcgacgaagcccagggcggcaccagcgaggatggatgggtggtgtgccgcgtgttcaagaagaagtgtttcttcaagatcgccggaggcggcgaagggagctcaagccagagcgcggacgccggcgccggccacatggccgtgtcgtcaccactgggtggccacgaccacgctgacagggccgccatggcatctcactacatgcacgggatgcacccgcagtaccacccccagcacgcctcttccttctactactcccagatgcaaccaccggaggcggcctactcgcaccacgtgcaggtccaggacctcctcaccaaccaccggccggcggccgacgcagctggcggcgggtatgatttctccggcctgccggtcgagcaccctggcctggacgtcggcagcagcgacggcgtcgcggcggatgggctggccgaaggaagagatcaaacaaacggtgcagccgatcagcagtggcagccgatggacgggttcagcaacggcgggggcgccgctgcagtacagcagatgagcgccatgaactccgggcaacgaggtggggagatggatttgtggggttatgggaggtaaattcaacatcaccaagacatgaaaaagtgcatctgcatgcatgaatgttgcatgcaagtgggaaggaggatcctgaaaatactggtaatgttgttggagagttatcattggtttttggtgtactgtatat

SEQ ID NO: 134 - protein, 336 aa, Triticum aestivum
MADFSSSNGVPPGFRFHPTDEELLLYYLKKKIGFEKFDLEVIREVDLNKIEPWDLQERCRIGSAPQNEWYFFSHKDRKYPTGSRTNRATTAGFWKATGRDKCIRTSYRKIGMRKTLVFYRGRAPHGQKSDWIMHEYRLEEIDEAQGGTSEDGWVVCRVFKKKCFFKIAGGGEGSSSQSADAGAGHMAVSSPLGGHDHADRAAMASHYMHGMHPQYHPQHASSFYYSQMQPPEAAYSHHVQVQDLLTNHRPAADAAGGGYDFSGLPVEHPGLDVGSSDGVAADGLAEGRDQTNGAADQQWQPMDGFSNGGGAAAVQQMSAMNSGQRGGEMDLWGYGR

SEQ ID NO: 135 - DNA, 1063 bp, Triticum aestivum
  misc_feature (706)..(706): n is a, c, g, or t
gcacacatgcatctactagtagtaagttgaaaaggttgtgcgcatgatttcagtttcgaagacatcaagctagcttgctaccaggcaggaaggtagatgctagcaatggaccagcacaaccatcaggaggagtcgtgtgtcccaccggggttcaggttccaccccactgaggaggagctggtggggtactacctcgccaggaaggtggctgcccaaaagatcgacctcgacatcatccaggaggttgatctctaccggatcgagccatgggacctccaagagaggtgcggcggcggcaggggaggacggggagcgcgtcaggtggcggcggaggacgagcagtcgtcggagtggtacttctttagcttcaaggatcgcaagtaccccagcggcacgcgcaccaaccgcgccacggcggccgggttctggaaggccaccggcagggacaagcccgtgacatcgtcgagcagccgaggggtcatcggcatgaggaagacgcttggtgttctacaggggccgtgcacccaacggtaggaagactgactggatcatccacgagtatcgcctgcagaccagcgagcacgcgcccacccaggaggaaggctgggtggtgtgccgggcgttccagaagccaaccccgaaccagaggccgtcgtacatcttcccggcatacgccgccgctccgggcctcgggggctactacgangcgcggccatggctgcacggncaaggcggcgacctccattacctgcaaagcgccacatgcgctgccggagccggcggctcggcttccccggccaaggcatcaagtactctgacgactgctggagtcaagcagagtctcttcggcaacatccgcactcatcgagagccgccgacaacaacgccgttgccggtggcggcgacgcggctatatgtctgcaacaggacagcancgccgnatcattggacttctgannctgtatcactccanctgactaaacatgaatnagtnacaacatacgtncanatcattcatgnnatatttcaatattnacacattntaaatanaatgntcanccat

SEQ ID NO: 136 - protein, 145 aa, Triticum aestivum
MLAMDQHNHQEESCVPPGFRFHPTEEELVGYYLARKVAAQKIDLDIIQEVDLYRIEPWDLQERCGGGRGGRGARQVAAEDEQSSEWYFFSFKDRKYPSGTRTNRATAAGFWKATGRDKPVTSSSSRGVIGMRKTLGVLQGPCTQR

SEQ ID NO: 137 - DNA, 710 bp, Zea mays
caagaactcatatatgcgcacaacactcttcctctgacaagagtttgttctgcaactaaccactatatatatagatcgatcctctatctgacaaggtgcaggtgcaggtgatcatatgaactccatggaatcatgcgtgcccccgggtttcaggttccaccccaccgacgaggagctcgtcggctactacctcaggaagaaggtggccgcccagaagatcgacctccacgtcatacgcgacatcgacctctaccgcatcgagccatgggatctccaagggcattgcgggatcgggtacgaggagcaaaacgagtggtacttcttcagctacaaggaccgcaagtaccccacggggacgcggaccaaccgggccaccatggccggcttctggaaggccacgggcagggacaaggcggtgcacgacaagagccgcatcatcggcatgaggaagacgctcgtcttctacaaggggagggcccccaacggccacaagaccgactggatcatgcacgagtaccgcctcgagaccgacgagaatgcgccgccgcaggaagaaggctgggtggtgtgccgagcgttcaagaagagaacagcgtacccggcaaggatggcatggggggaccccagctacgcctaccgcgagatcagcgccatgggcgcggcggcggcggcgttcgtggacccctaacgcggcggcgtacccgcagatc

SEQ ID NO: 138 - protein, 190 aa, Zea mays
MNSMESCVPPGFRFHPTDEELVGYYLRKKVAAQKIDLHVIRDIDLYRIEPWDLQGHCGIGYEEQNEWYFFSYKDRKYPTGTRTNRATMAGFWKATGRDKAVHDKSRIIGMRKTLVFYKGRAPNGHKTDWIMHEYRLETDENAPPQEEGWVVCRAFKKRTAYPARMAWGDPSYAYREISAMGAAAAAFVDP

SEQ ID NO: 139 - DNA, 837 bp, Zea mays
gtcttcctcccctcacatcctctggcctctggtcctccaccgtcctgctagccagagctgctcttgtacgcgcagctggcctctgtgcatatcaccagcacaccgcgcaggggaaggaaattaacaagagaaaaggcaaggagagcaggcaggcaaggaagctgcagaagccaaggaaggagaaggaggatcatcaatgagcatctcggtgaacgggcagtcgtgcgtgccgccggggttccgcttccaccccacggaggaggagctgctcaactactacctccgcaagaaggtggcctcccaggagatcgacctcgacgtcatccgcgacgtcgacctcaacaagctcgagccatgggacatccaagagaaatgcaagatcgggtcgggtccccagaacgactggtacttcttcagccacaaggacaagaagtacccgacggggacgcgcaccaaccgcgccacggccgccgggttctggaaggccaccggccgcgacaaggccatctacaacgccgtcaagcgcatcggcatgcgcaagacgctcgtcttctacaagggccgcgcgccgcacggccagaagtccgactggatcatgcacgagtaccgcctcgacgaccccgctgctgctgctgctgctggatccggtgatgccgtggccaacgacgacgcagccgccacggtaagcaaagcaacgaccctgatcgccgttaatctcttctctgcaccaccagttcacgtacgccaccattaataattgcctgccgtaagataagaaacaattatatggcggtggtggtgcaatcatgcgagtacggcgaccccgccttgatttga

SEQ ID NO: 140 - protein, 185 aa, Zea mays
MSISVNGQSCVPPGFRFHPTEEELLNYYLRKKVASQEIDLDVIRDVDLNKLEPWDIQEKCKIGSGPQNDWYFFSHKDKKYPTGTRTNRATAAGFWKATGRDKAIYNAVKRIGMRKTLVFYKGRAPHGQKSDWIMHEYRLDDPAAAAAAGSGDAVANDDAAATVSKATTLIAVNLFSAPPVHVRHH

SEQ ID NO: 141 - DNA, 1433 bp, Zea mays
atgcatccaggaggtggaccactgtcggtgccaccaggcttccgcttccacccgaccgacgaggagctcctctactactacctgaggaagaaggtttcctatgagcccatagatcttgatgtcataagggagattgatctcaaccagcttgagccatgggatctcaaaggtaggtgcaggataggcaccgggcatcagaacgagtggtacttcttcagccacaaggacaagaagtacccgacgggaacaaggacgaaccgcgccacgacggcggggttctggaaggcgactgggcgagacaaggccatcttcctcggcaacgcctccaggaggatcggcttgaggaagaccctggtgttctacatcggcagggcgccacacggcaagaagactgactggatcatgcacgagtaccgcctggaccaagagaacgttgagattcagggtctacaggaagatggttgggtggtgtgtagggttttcaagaagaagagctatcaccagagaggcctcaatccggctgaaatggccgccgcactggacgacgacgagctccagcctttccctgttcctatccccggcagctccctgcagccaacagagcacaagaccaaccctactcacctcatgcaatatgactatccttccttcgacccttccatgcagctcccacagctgatgagcgatgaccaggaacaggaacagcccgtgcggccaaccctcctccccggccatcccggcgttgctatggcctccatgagctcactcaacattgagtgctcgcagaacctgacgaagctcacatctaacggcagcgacgagatgctccacgtcggccacggcggtggggctggaggtggtgtagtcgtcgaccgcttcgctggcacgacgacagattggtcgatcctagacaagctcctcgcctcgcaccagaacctcgaccggctcttccacggcaaggtcgtcaccacggcatctgcctccccaatggcggtaccggtataccatcagcagctcatggaactcggtggctcgtcgtcgtccttgcagaggcttccactgcagtacctcggcggcgagacggccgatctcctcaggtaccccaagtaattggatccacgtcaagtttagctacctcttgttgagcagtgacctgatcagggtttttggttgactactagatcgatcgattgcttaattaattcggtctactttgtatggagagtttagtgctcctaaatgtatatatatggtgcaactgttttggggcatgcaggcatggaatgccatctactgtggttgattgtttggtcaggactcaggaggatctttttgctgttttcattttggcttagggttgcttgttaggtgcttttggtggaaaaaatgtgaagagatggcatggtcagtcaagaacttctatttgtttcctc

SEQ ID NO: 142 - protein, 369 aa, Zea mays
MHPGGGPLSVPPGFRFHPTDEELLYYYLRKKVSYEPIDLDVIREIDLNQLEPWDLKGRCRIGTGHQNEWYFFSHKDKKYPTGTRTNRATTAGFWKATGRDKAIFLGNASRRIGLRKTLVFYIGRAPHGKKTDWIMHEYRLDQENVEIQGLQEDGWVVCRVFKKKSYHQRGLNPAEMAAALDDDELQPFPVPIPGSSLQPTEHKTNPTHLMQYDYPSFDPSMQLPQLMSDDQEQEQPVRPTLLPGHPGVAMASMSSLNIECSQNLTKLTSNGSDEMLHVGHGGGAGGGVVVDRFAGTTTDWSILDKLLASHQNLDRLFHGKVVTTASASPMAVPVYHQQLMELGGSSSSLQRLPLQYLGGETADLLRYPK

SEQ ID NO: 143 - DNA, 1472 bp, Zea mays
atgcatccaggtgttggaccattgtcggtgccaccaggcttccgcttccatccgactgatgaggagctcctctactactacctgaggaaaaaggttgcttatgagcccatagatcttgatgtcataagggagattgatctcaataagcttgagccctgggatctcaaagaaagatgcaaaataggcactgggcctcaaaacgagtggtacttcttcagccacaaggacaagaagtactcaacgggaacgagaacgaaccgtgccacgacggcgggattctggaaggcgactggccgagacaaggccatcttccttggcagcgccaggaggatcggcttgagaaagaccctggtgttctatatcggcagggcgccgcacgggaagaagactgagtggatcatgcacgagtaccgccttgatgaagagaacgttgaaattcaggaagatggatgggtggtatgtagggttttcaagaagaagaactatgagcagagaggccacaacatggctgagatggcggcactggacgacgatgagctccagcctttcacggttcctgtcgtccctgctggcactagctccctgccaacaacagaccacaagaacaaccctcacctcatgcaatatgacttcccttctttcgacccttccatgcagctcccacagctgatgagcgccgaccagcccgtgccaaccctattccccagccatcctggcgtcgccacggccatgagctcatcaatcgacgttgagtgctcgcagaacctgctgacgatgctgacatccaacggcagcgacgggatgctccacgtccgcgctggtggtgctggagctggtgtcgaccgcttcgctggcacgacagattggtcgatcctagacaagctcctcgcctcgcaccagaacctcggacagctcttccacggcaaggtcaccgcagcatctgcctccccaatggctccataccatcagcagctcatggaactcgggtggctcgtcgtcgtcgtcgtccttgcagaggcttccactgcagtacctcagcggcgagacgaccgatctcctcaggttccccaagtaattattggatcgacctcaagtttagctaccttgttgagcagtgaccatatcaggattttggttgactactaggctgcttaattcggcctacttcgtatggagtttagtgcttttaaatgtatatatggtgcaactgtttggggcatgcaggcatggaatgccatcgatctaccatggttgcttgcttggccaagaggatgtttttgatgtttccattttggcttagggttgctcgttaggttcttttggagacaaaatgtgaagagatggcccggtcaattaagaacttctatttgtttcccgaacatattacttctgtttcaaactctagttcccacagattggtcttttggttgttcgttcaactataataatataggatgatggggcatttatcc

SEQ ID NO: 144 - protein, 381 aa, Zea mays
MHPGVGPLSVPPGFRFHPTDEELLYYYLRKKVAYEPIDLDVIREIDLNKLEPWDLKERCKIGTGPQNEWYFFSHKDKKYSTGTRTNRATTAGFWKATGRDKAIFLGSARRIGLRKTLVFYIGRAPHGKKTEWIMHEYRLDEENVEIQEDGWVVCRVFKKKNYEQRGHNMAEMAALDDDELQPFTVPVVPAGTSSLPTTDHKNNPHLMQYDFPSFDPSMQLPQLMSADQPVPTLFPSHPGVATAMSSSIDVECSQNLLTMLTSNGSDGMLHVRAGGAGAGVDRFAGTTDWSILDKLLASHQNLGQLFHGKVTAASASPMAPYHQQLMELGWLVVVVVLAEASTAVPQRRDDRSPQVPQVIIGSTSSLATLLSSDHIRILVDY

SEQ ID NO: 145 - DNA, 1291 bp, Zea mays
cttaacaactccactcccgcggaaaaaaaacccatatccctccggcataaacccaaaaagctgcaagcagtgtgaccaaaagcaggcctgacggcgatcctcggctctctagccaggcagcaggagatggagcacgacgtgcaccaccagcaccagcaggccatggagctgccgccggggttccgattccaccccaccgacgaggagctcatcacgcactacctcgccaggaaggccgccgacgcccgcttcgccccgcgcgccgtcggcgaggccgacctcaacaagtgcgagccatgggacctgccatcccgggcgacgatgggcgagaaggagtggtacttcttctgcgtcaaggaccgcaagtacccgacggggctgaggacgaaccgggccaccgagtccggatactggaaggcgacgggcaaggacagggagatcttcaggagcaaggccctcgtcggcatgaagaagacgctcgtcttctacacggggagggcgcccaggggaggcaagaccggctgggtcatgcacgagtaccgcctccacggcaagcacgccagcagcagccgcctcatgccgtcgtcggtcagagctggcgcgtcaaaggacgagtgggtgctgtgcagggtgttcaagaagagcatcgagccgccgccgtcagtgggcaagaggtcgtcggtcgcgtgcacggggacgatggtggtggaggacgtcgtgggaccgccgtccatgtccatggaggacgacctcaccgcgtgcgcgctgcctccgctgatggacgtgtccggcggtggcggcgccaacatggcggcggcgtccatcgagctgctggcgccaccggcaccacacgtgacctgcttttccaacgcgctggagggccagttcttcctgaacccaccctgcctccacccctccacgtcgccgctccgcctggcgagcatggtgcaggcgcaggcgcaggcgcagtacgacggcgacgcgggcatggtgctccagctcctgcaggaggccggcgggtggtacagcaagctgggcgagggggagcggctgagcggcggcgcgtcgcaggacaccggcgtcacctcggaggtgaacgtgaaccccgccgagatctcctcgacgcggcaccacataatggaccccgaggcggcctccttctggggcttttgaacttccccccacatctttttttttccgcgatcgctaaaaattggattagaattagtcactggattggaaactgggattgtaattgtgccgttgaatattactatatgcaaaatggggc

SEQ ID NO: 146 - protein, 348 aa, Zea mays
MEHDVHHQHQQAMELPPGFRFHPTDEELITHYLARKAADARFAPRAVGEADLNKCEPWDLPSRATMGEKEWYFFCVKDRKYPTGLRTNRATESGYWKATGKDREIFRSKALVGMKKTLVFYTGRAPRGGKTGWVMHEYRLHGKHASSSRLMPSSVRAGASKDEWVLCRVFKKSIEPPPSVGKRSSVACTGTMVVEDVVGPPSMSMEDDLTACALPPLMDVSGGGGANMAAASIELLAPPAPHVTCFSNALEGQFFLNPPCLHPSTSPLRLASMVQAQAQAQYDGDAGMVLQLLQEAGGWYSKLGEGERLSGGASQDTGVTSEVNVNPAEISSTRHHIMDPEAASFWGF

SEQ ID NO: 147 - DNA, 1529 bp, Zea mays
ccggctccggctccggcaccggctcaatacaatcccatcaccgatcattcattccctttgcctactactgctacctccccctccttccatcttcgatagacgaccaaataatggcggtattcgacgtctgaatcctcgtactgatcttatactgcatacagacagacagacccttcttcttccttagttccctatagctagctctagctaggagaccgatcatgagcatctcggtgaacgggcagtcggtggtgccgccggggttccgtttccacccgacggaggaggagctgctgacctactacctgaagaagaaggtggcgtcggagcgcatcgacctggacgtcatccgcgacgtcgacctgaacaagctggagccatgggacatccaagagaagtgccgcatcggttcgggcccccagaacgactggtacttcttcagccacaaggacaagaagtacccgacggggacgcgcaccaaccgcgccaccgccgccgggttctggaaggccaccggccgcgacaaggccatctacgcctcgccgggcgcccgccgcatcggcatgcgcaagacgctcgtcttctacaagggccgcgccccgcacgggcagaagtccgactggatcatgcacgagtaccgcctcgaggcgcccgtcgacgccggcgccggcgccgcgcaccacctgctgctccccgccgccgaacatcctccctactacacctcgccgccgcaggcgccttcttctactactaccgcaacgatccgtggcgcggcgggagaccaagcggcgcaggagcaggaggggtgggtgatctgcagggtgttcaagaagaagaacctcgtgcaccacggccagagcagcggcgtgaagcagcaggcagcaggagacgaccacgcggcgtcccatacggcggcggcggcagcgcacatggacgagagcagcccgagccagtgctcgtcggtgaccgtcatcagcgaccacgtgcacgccaacgttaacgacaagcagcagcaggcgcaggcgtcgttgctgatgatgcacacgcaccactccgccagcagcgacgacgacgcgctggaccacatcctgcagcagtacatgggcggcggcaggcaggcgcccgcgccggacaccaagccggcgctgctggagcagctggaccatctgcaccaccaccttgccgcagcgcctactactagggccgccgccggcttctactacgggaagttcatgaagctcccgcccctcgagcacgccggcctgccgccaagcccaccaccaccaggagcgcgcgagtacggcgccgccgccgccgccggctgggacgacgacgacgacgcgctggaccgcctcgccgcctacgaccacctcaacggcctctccaacgacgcgtccaagaacatggccgcgttcttcgacgtcgagcctagcgccgccgccgccgccgccgtggacggcgacctgtggagcctcgcacggtccgtgtcggcgctgcacgcggacttgaccatgaacaacaacgtctag

SEQ ID NO: 148 - protein, 435 aa, Zea mays
MSISVNGQSVVPPGFRFHPTEEELLTYYLKKKVASERIDLDVIRDVDLNKLEPWDIQEKCRIGSGPQNDWYFFSHKDKKYPTGTRTNRATAAGFWKATGRDKAIYASPGARRIGMRKTLVFYKGRAPHGQKSDWIMHEYRLEAPVDAGAGAAHHLLLPAAEHPPYYTSPPQAPSSTTTATIRGAAGDQAAQEQEGWVICRVFKKKNLVHHGQSSGVKQQAAGDDHAASHTAAAAAHMDESSPSQCSSVTVISDHVHANVNDKQQQAQASLLMMHTHHSASSDDDALDHILQQYMGGGRQAPAPDTKPALLEQLDHLHHHLAAAPTTRAAAGFYYGKFMKLPPLEHAGLPPSPPPPGAREYGAAAAAGWDDDDDALDRLAAYDHLNGLSNDASKNMAAFFDVEPSAAAAAAVDGDLWSLARSVSALHADLTMNNNV

SEQ ID NO: 149 - DNA, 1395 bp, Zea mays
gtcgtcgtcgcgcgcgcgctgaatctgatgcacgcacatactactacgcaggtgcaggtgcaggtgatcatatgaactccatggaatcatgcgtgcccccgggtttcaggttccaccccaccgacgaggagctcgtcggctactacctcaggaagaaggtggccgcccagaagatcgacctccacgtcatacgcgacatcgacctctaccgcatcgagccatgggatctccaagggcattgcgggatcgggtacgaggagcaaaacgagtggtacttcttcagctacaaggaccgcaagtaccccacggggacgcggaccaaccgggccaccatggccggcttctggaaggccacgggcagggacaaggcggtgcacgacaagagccgcatcatcggcatgaggaagacgctcgtcttctacaaggggagggcccccaacggccacaagaccgactggatcatgcacgagtaccgcctcgagaccgacgagaatgcgccgccgcaggaagaaggctgggtggtgtgccgagcgttcaagaagagaacagcgtacccggcaaggatggcatggggggaccccagctacgcctaccgcgagatcagcgccatgggcgcggcggcggcggcgttcgtggaccctaacgcggcgtcgtacgcgcagatcagacgccagcagtccaacaagagcgcgcgcttcaagcaggagggcgagctggacggcggcgccgccgccgcgctgctgcagtactcctccagccacctggtcgagctgccgcagctcgagagcccgtcggcgccggccaacaagagccaggcggcgtcagcctctgatgaggttgtggacgccgccgacagcggccggcggccgggcaagaaggcgcgggctgatgaagtggccacggactggagggcgctcgacaagttcgtcgcgtcgcagctcagccccgcggcggagtgcggcttggaggcggcggcagcgtcgtcgacggccgccgccagcaacgtgggctcccagctggaccacgttgaggatgatgacatggcggcattgctgttcctcaacagcgacgggagagacgaggcggagaggtggacggggctgctcggcccggccgccggggacgacggcgactttggactgtgtgtgttcgacaaatgagtggcgggcgcggacggccggcgcgccgcccgcgtgcatggtgtggcgcggagggatgcatgcatgcatgcgtccatgggatggtgcgtgtcatatattatgtggttatagctgaaagttggtctctcttttattttttttccttgactactttccctttgtatgtttgccaacaaatgtgattgtaattttcatctgtctctgtgcagagtcataagatgcatgccattccggc

SEQ ID NO: 150 - protein, 362 aa, Zea mays
MNSMESCVPPGFRFHPTDEELVGYYLRKKVAAQKIDLHVIRDIDLYRIEPWDLQGHCGIGYEEQNEWYFFSYKDRKYPTGTRTNRATMAGFWKATGRDKAVHDKSRIIGMRKTLVFYKGRAPNGHKTDWIMHEYRLETDENAPPQEEGWVVCRAFKKRTAYPARMAWGDPSYAYREISAMGAAAAAFVDPNAASYAQIRRQQSNKSARFKQEGELDGGAAAALLQYSSSHLVELPQLESPSAPANKSQAASASDEVVDAADSGRRPGKKARADEVATDWRALDKFVASQLSPAAECGLEAAAASSTAAASNVGSQLDHVEDDDMAALLFLNSDGRDEAERWTGLLGPAAGDDGDFGLCVFDK

SEQ ID NO: 151 - DNA, 1548 bp, Zea mays
aaagcagcagctgctcttctcttctgctagcagctttatttgcctgcctccctaccggcgagggtatcgatctgtactctctatcctttctttgcatgcatgtgcagattgcagtgtcttctttcttgctatagcttcctccgatccctgaccctgatcctgatgactcactacaaggaggtacgtgcgccgatcgagaagcagcagcagcaattaataactaaacatggatcaggcggcggaggagtcttgtgttcccccaggcttcaggttccaccctacggaggaagaactggtgggctactaccttgccaggaaggtggcctcccagaggatcgacctcgacatcatccaggaggtggatctctacaggatagagccatgggatctgcaagagcggtgcaagccgcagcacgccggcggtgggcacgacgagcagacacaggagtggtacttcttcagctacaaggaccgcaagtaccccagcgggacgaggacgaaccgcgccacggcggccggcttctggaaggccaccggcagggacaaaccggtgctgtcgtcgtccaccaggaccaccaggtgtagcagggtcatcggcatgaggaagacgctggtgttctacaagggcagggcacccaacggcaggaagagcgactggatcatgcacgagtaccgactccaatccaacgagcacgcactggcgcaggaggaaggctgggtggtgtgccgcgctttccagaagcctatcccctaccagcggcccttcttccccaccagctacgccggctactacgaccacaacctctcaacgacggcacggctgcacgtagacggcgaccgccatttcctggcgggctcatcagcagcagcagcagcactgctacagcagccaccagggcttgcaggcagcagcttcccgcagctgtactccgacgacgacttggagtccaagaagcagctgttgagtatcccgccgctggagagccccactgccatggcctgctgttccgacgccggcggctacgcgcagagaagcagctacgatgaacatgagatgatgatgatgatgatccagcagggaggaggaggaggagagcaagctgcagccgccatcgactggaactttctggacagcctgctgtccgcgacgtcgcaactccatgggccctcgggatccttgttgcagtgacctccggccatcgtcgatcgatccatctttctttgatccagcggagaacattattcattcatcggcattatacaatatatttattagttgcatacacacacagagtatacgtatatgctaataattcagatgtgttcatacacacttccattgtcatgcatgtgtagttcgttgcgacttacattacagactgccttagatgattcaggtatactgtatacaacatgatgcacagtatagtaggagtaagtgcaccagtctcagcagctggtagctagctagctatgtaaaattcctccttaattacagctatgtaacatatatatacagttattattacttactgatatgatctatct

SEQ ID NO: 152 - protein, 320 aa, Zea mays
MDQAAEESCVPPGFRFHPTEEELVGYYLARKVASQRIDLDIIQEVDLYRIEPWDLQERCKPQHAGGGHDEQTQEWYFFSYKDRKYPSGTRTNRATAAGFWKATGRDKPVLSSSTRTTRCSRVIGMRKTLVFYKGRAPNGRKSDWIMHEYRLQSNEHALAQEEGWVVCRAFQKPIPYQRPFFPTSYAGYYDHNLSTTARLHVDGDRHFLAGSSAAAAALLQQPPGLAGSSFPQLYSDDDLESKKQLLSIPPLESPTAMACCSDAGGYAQRSSYDEHEMMMMMIQQGGGGGEQAAAAIDWNFLDSLLSATSQLHGPSGSLLQ

SEQ ID NO: 153 - DNA, 1246 bp, Zea mays
ctctccagcacaaacctcccctcctcaagctgtttattctccccaaaacaagccggctagctagcgctcttccgagagatcaagaacacatgcgcacactctttctctgacaagagcttgtgttctgcaactgaccagtatatacatcgaacctctatctaacaaggtgcaggcgcagggtgatcatatggaatcatgcgtgcccccggggttcaggttccaccccaccgacgaggagctcgtcggctactacctccggaagaaggtggcctcccagaagatcgacctcgacgtcatacgcgacatcgacctctaccgcatcgagccgtgggatctccaagaacactgcgggatcggccggtacgaggagcagagcgagtggtacttcttcagctacaaggaccgcaagtacccgacggggacgcggaccaacagggccaccatggcagggttctggaaggccacgggcagggacaaggcggtgcacgacaggagccgcctcatcggcatgaggaagacgctcgtcttctacaaggggagggcgcccaacggccagaagaccgactggatcatgcacgagtaccggctcgagaccgacgagaacgcgcagccacaggcaagcaaatgatctcacctgcaggctgcagcttactcatctacctttaatcacccgcgcttattagtatcactattaagcacgctactcttctgtttaaaatctattctgaatctctcttctgctaaaaatttatttatttatctatatgataaagcttaatgatgtctcagttacagagaccaagagatcgatctctctttaaagtattgcgcacaactgctaatatagcaaaattggagagaccaatagcaaataaagcagcacaagcacagttgctaatcgctttctagctctcagaagaaacttccaaagacttcaattcttcttctcagtaaagttaggcagcatatatatgcaggcctctcaccaatcacaaaaacttttcctatatagatgcatgcatgcatgtcccaattagggtttcccagtgtgaaccttctattgcacgcgcaaatgtttaatttaccagctgtatgcaactagctgtactgctggcatactcgtcaaagtaccgatggtaatgcttcgttcggcactccaccactatcgcacacagtacggagataggcgataataaagccaaacgttctgcttaataaccttttgtaatcaattcaatgccggccg

SEQ ID NO: 154 - protein, 146 aa, Zea mays
MESCVPPGFRFHPTDEELVGYYLRKKVASQKIDLDVIRDIDLYRIEPWDLQEHCGIGRYEEQSEWYFFSYKDRKYPTGTRTNRATMAGFWKATGRDKAVHDRSRLIGMRKTLVFYKGRAPNGQKTDWIMHEYRLETDENAQPQASK

SEQ ID NO: 155 - protein, 11 aa, Artificial Sequence
  Motif I
KIDLDIIQELD

SEQ ID NO: 156 - protein, 15 aa, Artificial Sequence
  Motif II
CKYGXGHGGDEQTEW

SEQ ID NO: 157 - protein, 11 aa, Artificial Sequence
  Motif III
GWVVCRAFQKP

(19) United States
(12) Patent Application Publication
(10) Pub. No.: US 2012/0137386 A1
(43) Pub. Date: May 31, 2012

(54) Plants Having Modulated Carbon Partitioning and a Method for Making the Same

(75) Inventors: Chang-Deok Han, Jinju (KR); Su Hyun Park, Jinju Kyungman (KR); Yand Do Choi, Seoul (KR)

(73) Assignees: Crop Functional Genomics Center, Seoul (KR); BASF Plant Science Company GmbH, Ludwigshafen (DE)

(21) Appl. No.: 13/382,242

(22) PCT Filed: Jul. 1, 2010
(86) PCT No.: PCT/EP2010/059339

§ 371 Date: Feb. 8, 2012

(30) Foreign Application Priority Data
Jul. 7, 2009 (EP) 09164829.5

(51) Int. Cl. A01H 1/00; C12N 5/10; A01H 5/10; A01N 43/54; A01H 5/00; C12N 5/04; C12N 15/82

(52) U.S. Cl. 800/278; 435/410; 435/419; 435/320.1; 504/240; 800/298; 800/320; 800/320.1; 800/320.2; 800/320.3

(57) ABSTRACT

The present invention relates generally to the field of molecular biology and concerns a method for modulating carbon partitioning in plants. More specifically, the present invention concerns a method for modulating carbon partitioning in plants by modulating expression in a plant of a nucleic acid encoding a particular type of NAC transcription factor. The present invention also concerns plants having modulated expression of a nucleic acid encoding a NAC transcription factor, which plants have modulated carbon partitioning relative to control plants. The invention also provides constructs useful in the methods of the invention.

The present invention relates generally to the field of molecular biology and concerns a method for modulating carbon partitioning in plants. More specifically, the present invention concerns a method for modulating carbon partitioning in plants by modulating expression in a plant of a nucleic acid encoding a particular type of NAC transcription factor. The present invention also concerns plants having modulated expression of a nucleic acid encoding a NAC transcription factor, which plants have modulated carbon partitioning relative to control plants. The invention also provides constructs useful in the methods of the invention.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic and horticultural traits.

The present invention concerns the use of a particular type of NAC transcription factor for modulating carbon portioning in plants. Transcription factors are usually defined as proteins that show sequence-specific DNA binding and that are capable of activating and/or repressing transcription. The Arabidopsis genome codes for at least 1533 transcriptional regulators, which account for ˜5.9% of its estimated total number of genes. About 45% of these transcription factors are reported to be from families specific to plants (Riechmann et al., 2000 (Science Vol. 290, 2105-2109)). One example of such a plant-specific family of transcription factors is the family of NACs.

NAC is an acronym derived from the names of the three genes first described as containing a NAC domain, namely NAM (no apical meristem), ATAF1,2 and CUC2 (cup-shaped cotyledon). NAC proteins appear to be widespread in plants, with the genome of Arabidopsis thaliana estimated to contain at least a hundred NAC-encoding genes, but without any examples having been found to date in other eukaryotes (Riechmann et al., 2000).

The NAC protein family comprises a variety of plant proteins that are identifiable by the presence of a highly conserved N-terminal NAC domain, accompanied by diverse C-terminal domains. The DNA-binding ability of NAC proteins is generally localized to the NAC domain, with the C-terminal regions constituting transcriptional activation domains. Several NAC genes have been found to be hormone inducible. NAC domains have also been implicated in interactions with other proteins, such as viral proteins and RING finger proteins. NAC proteins have also been implicated in transcriptional control in a variety of plant processes, including in the development of the shoot apical meristem and floral organs, and in the formation of lateral roots. NAC proteins have also been implicated in responses to stress and viral infections, Ernst et al., 2004 (EMBO Reports 5, 3, 297-303).

U.S. Pat. No. 6,844,486 describes a member of the NAC family, NACl, isolated from Arabidopsis thalianana. NACl was reported to be involved in the regulation of cotyledon and lateral root development. Overexpression of the nacl gene was reported to give larger plants with larger roots and more lateral roots than wild-type plants.

Surprisingly, it has now been found that modulating expression in a plant of a nucleic acid encoding a particular type of NAC transcription factor gives plants having modulated carbon partitioning relative to control plants. The particular class of NAC transcription factor suitable for modulating carbon partioning in plants is described in detail below.

The present invention provides a method for modulating carbon partitioning in plants relative to control plants, comprising modulating expression in a plant of a nucleic acid encoding a particular type of NAC transcription factor.

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. A “control plant” as used herein refers not only to whole plants, but also to plant parts, including seeds and seed parts.

A preferred method for modulating (preferably, increasing) expression of a nucleic acid encoding a NAC transcription factor is by introducing and expressing in a plant a nucleic acid encoding a particular class of NAC transcription factor as further defined below.

The nucleic acid to be introduced into a plant (and therefore useful in performing the methods of the invention) is any nucleic acid encoding the type of NAC transcription factor which will now be described. A “NAC transcription factor” as defined herein refers to any amino acid sequence which when used in the construction of a NAC phylogenetic tree, such as the one depicted in FIG. 1, tends to cluster with the group of NACs comprising the amino acid sequence represented by SEQ ID NO: 2 rather than with any other NAC group.

A person skilled in the art could readily determine whether any amino acid sequence in question falls within the definition of a “NAC transcription factor” using known techniques and software for the making of such a phylogenetic tree, such as a GCG, EBI or CLUSTAL package, using default parameters. The phylogenetic tree of FIG. 1 is taken from Ooka et al., 2003 (DNA Research 10, 239-247, 2003). The method for constructing such a phylogenetic tree is described in Ooka et al., 2003. In the phylogenetic tree of FIG. 1, SEQ ID NO: 2 is found in the group third up from the bottom, labelled “OsNAC7”. Any sequence clustering within this group comprising SEQ ID NO: 2 would be considered to fall within the aforementioned definition of a NAC transcription factor, and would be considered suitable for use in the methods of the invention.

Additionally or alternatively, a “NAC transcription factor” as defined herein is one comprising any one or more of the Motifs described below.

Motif I: KIDLDIIQELD, or a motif having in increasing order of preference at least 50%, 60%, 70%, 80% or 90% sequence identity to the sequence of Motif I.

Motif I is preferably K/P/R/G I/S/M D/A/E/Q L/I/V D I/V/F I Q/V/R/K E/D L/I/V D.

Motif II: CKYGXGHGGDEQTEW, or a motif having in increasing order of preference at least 50%, 60%, 70%, 80% or 90% sequence identity to the sequence of Motif II, where ‘X’ is taken to be any amino acid or a gap.

Motif II is preferably C K/R Y/L/I G XXX G/Y/N D/E E Q/R T/N/S EW, where ‘X’ is any amino acid or a gap.

Motif III: GWVVCRAFQKP, or a motif having in increasing order of preference at least 50%, 60%, 70%, 80% or 90% sequence identity to the sequence of Motif III.

Motif III is preferably GWVVCR A/V F X¹ K X², where ‘X¹’ and ‘X²’ may be any amino acid, preferably X¹ is Q/R/K, preferably X² is P/R/K.

Motifs I to III are found in the NAC represented by SEQ ID NO: 2 and are also typically found in NACs clustering (in a phylogenetic tree of NACs) with the group of NACs comprising SEQ ID NO: 2 rather than with any other NAC group.

Each of Motifs I to III may comprise one or more conservative amino acid substitution at any position.

Examples of NAC transcription factors as defined herein, i.e. any amino acid sequence which when used in the construction of a NAC phylogenetic tree, such as the one depicted in FIG. 1, tends to cluster with the group of NACs comprising the amino acid sequence represented by SEQ ID NO: 2 rather than with any other NAC group, are as given in Table 1 below.

TABLE 1
Examples of NAC transcription factors that cluster (in a phylogenetic
tree) with the NAC represented by SEQ ID NO: 2 rather than with any
other NAC group

| | | NCBI | Other | |
|---|---|---|---|---|
| | | Accession | name | |
| Nucleic Acid | Amino Acid | Number | (if any) | Source |
| SEQ ID NO: | SEQ ID NO: | XM_479673.1 | NAM2 | Oryza sativa |
| 1 | 2 | | (also called | |
| | | | CDS4054 | |
| | | | in the | |
| | | | alignment | |
| | | | of FIG. 2) | |
| SEQ ID NO: | SEQ ID NO: | AK106313 | | Oryza sativa |
| 3 | 4 | | | |
| SEQ ID NO: | SEQ ID NO: | NM_187584.1 | | Oryza sativa |
| 5 | 6 | | | |
| SEQ ID NO: | SEQ ID NO: | XM_467007.1 | | Oryza sativa |
| 7 | 8 | | | |
| SEQ ID NO: | SEQ ID NO: | XM_473322.1 | | Oryza sativa |
| 9 | 10 | | | |
| SEQ ID NO: | SEQ ID NO: | ABE89364 | | Medicago |
| 11 | 12 | | | truncatula |
| SEQ ID NO: | SEQ ID NO: | NM_101098.2 | ANAC007/ | Arabidopsis |
| 13 | 14 | | EMB2749 | thaliana |
| SEQ ID NO: | SEQ ID NO: | NM_104947.1 | ANACO26 | Arabidopsis |
| 15 | 16 | | | thaliana |
| SEQ ID NO: | SEQ ID NO: | XM_476289.1 | | Oryza sativa |
| 17 | 18 | | | |
| SEQ ID NO: | SEQ ID NO: | NM_127362.1 | ANAC037 | Arabidopsis |
| 19 | 20 | | | thaliana |
| SEQ ID NO: | SEQ ID NO: | NM_119783.2 | ANAC076 | Arabidopsis |
| 21 | 22 | | | thaliana |
| SEQ ID NO: | SEQ ID NO: | NM_187584.1 | | Oryza sativa |
| 23 | 24 | | | |
| SEQ ID NO: | SEQ ID NO: | NM_125632.1 | ANAC101 | Arabidopsis |
| 25 | 26 | | | thaliana |
| SEQ ID NO: | SEQ ID NO: | NM_126028.1 | ANAC105 | Arabidopsis |
| 27 | 28 | | | thaliana |
| SEQ ID NO: | SEQ ID NO: | AK071064.1 | | Oryza sativa |
| 29 | 30 | | | |
| SEQ ID NO: | SEQ ID NO: | NM_105851.1 | ANAC030 | Arabidopsis |
| 31 | 32 | | | thaliana |
| SEQ ID NO: | SEQ ID NO: | NM_197511.1 | | Oryza sativa |
| 33 | 34 | | | |
| SEQ ID NO: | SEQ ID NO: | AB217775.1 | | Zinnia elegans |
| 35 | 36 | | | |
| SEQ ID NO: | SEQ ID NO: | AJ833965.1 | | Zea mays |
| 37 | 38 | | | |

The invention is illustrated by transforming plants with the Oryza sativa sequence represented by SEQ ID NO: 1, encoding the polypeptide sequence of SEQ ID NO: 2, however performance of the invention is not restricted to these sequences. The methods of the invention may advantageously be performed using any nucleic acid encoding a NAC transcription factor as defined herein, such as any of the nucleic acid sequences given in Table 1.

The NAC amino acid sequences given in Table 1 may be considered to be orthologues and paralogues of the NAC represented by SEQ ID NO: 2. Orthologues and paralogues encompass evolutionary concepts used to describe the ancestral relationships of genes. Paralogues are genes within the same species that have originated through duplication of an ancestral gene and orthologues are genes from different organisms that have originated through speciation.

Orthologues and paralogues may easily be found by performing a so-called reciprocal blast search. Typically, this involves a first BLAST involving BLASTing a query sequence (for example using any of the sequences listed in Table 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 1 or SEQ ID NO: 2, the second BLAST would therefore be against Oryza sequences). The results of the first and second BLASTs are then compared. A paralogue is identified if a high-ranking hit from the first blast is from the same species as from which the query sequence is derived, a BLAST back then ideally results in the query sequence as highest hit; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Table 1 gives examples of orthologues and paralogues of the NAC represented by SEQ ID NO 2. Further orthologues and paralogues may readily be identified using the BLAST procedure described above.

The NAC proteins are identifiable by the presence of a highly conserved N-terminal NAC domain (shown in FIG. 2), accompanied by diverse C-terminal domains. The term “domain” refers to a set of amino acids conserved at specific positions along an alignment of sequences of evolutionarily related proteins. While amino acids at other positions can vary between homologues, amino acids that are highly conserved at specific positions indicate amino acids that are essential in the structure, stability or activity of a protein. Identified by their high degree of conservation in aligned sequences of a family of protein homologues, they can be used as identifiers to determine if any polypeptide in question belongs to a previously identified polypeptide family (in this case, the family of NAC transcription factors).

Specialist databases also exist for the identification of domains. The NAC domain in a NAC transcription factor may be identified using, for example, SMART (Schultz et al. (1998) Proc. Natl. Acad. Sci. USA 95, 5857-5864; Letunic et al. (2002) Nucleic Acids Res 30, 242-244, InterPro (Mulder et al., (2003) Nucl. Acids. Res. 31, 315-318, Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAIPress, Menlo Park; Hulo et al., Nucl. Acids. Res. 32:D134-D137, (2004), or Pfam (Bateman et al., Nucleic Acids Research 30(1): 276-280 (2002).

NAC domains may also be identified using routine techniques, such as by sequence alignment. Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (over the whole the sequence) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Minor manual editing may be performed to optimise alignment between conserved motifs, as would be apparent to a person skilled in the art.

Nucleic acids encoding NAC transcription factors defined herein need not be full-length nucleic acids, since performance of the methods of the invention does not rely on the use of full length nucleic acid sequences. Examples of nucleic acids suitable for use in performing the methods of the invention include the nucleic acid sequences given in Table 1, but are not limited to those sequences. Nucleic acid variants may also be useful in practising the methods of the invention. Examples of such nucleic acid variants include portions of nucleic acids encoding a NAC transcription factor as defined herein, splice variants of nucleic acids encoding a NAC transcription factor as defined herein, allelic variants of nucleic acids encoding a NAC transcription factor as defined herein and variants of nucleic acids encoding a NAC transcription factor as defined herein that are obtained by gene shuffling. The terms portion, splice variant, allelic variant and gene shuffling will now be described.

According to the present invention, there is provided a method for modulating carbon partitioning in plants, comprising introducing and expressing in a plant a portion of any one of the nucleic acid sequences given in Table 1, or a portion of a nucleic acid encoding an orthologue, paralogue or homologue of any of the amino acid sequences given in Table 1.

Portions useful in the methods of the invention, encode a polypeptide falling within the definition of a NAC transcription factor as defined herein and having substantially the same biological activity as the NAC transcription factor represented by any of the amino acid sequences given in Table 1. Preferably, the portion is a portion of any one of the nucleic acids given in Table 1. The portion is typically at least 600 consecutive nucleotides in length, preferably at least 700 consecutive nucleotides in length, more preferably at least 800 consecutive nucleotides in length and most preferably at least 900 consecutive nucleotides in length, the consecutive nucleotides being of any one of the nucleic acid sequences given in Table 1. Most preferably the portion is a portion of the nucleic acid of SEQ ID NO: 1. Preferably, the portion encodes an amino acid sequence comprising any one or more of Motifs I to III as defined herein. Preferably, the portion encodes an amino acid which when used in the construction of a NAC phylogenetic tree, such as the one depicted in FIG. 1, tends to cluster with the group of NACs comprising the amino acid sequence represented by SEQ ID NO: 2 rather than with any other NAC group.

A portion of a nucleic acid encoding a NAC transcription factor as defined herein may be prepared, for example, by making one or more deletions to the nucleic acid. The portions may be used in isolated form or they may be fused to other coding (or non coding) sequences in order to, for example, produce a protein that combines several activities. When fused to other coding sequences, the resultant polypeptide produced upon translation may be bigger than that predicted for the NAC transcription factor portion.

Another nucleic acid variant useful in the methods of the invention is a nucleic acid capable of hybridising, under reduced stringency conditions, preferably under stringent conditions, with a nucleic acid encoding a NAC transcription factor as defined herein, or with a portion as defined herein.

Hybridising sequences useful in the methods of the invention, encode a polypeptide having a NAC domain (see the alignment of FIG. 2) and having substantially the same biological activity as the NAC transcription factor represented by any of the amino acid sequences given in Table 1. The hybridising sequence is typically at least 600 consecutive nucleotides in length, preferably at least 700 consecutive nucleotides in length, more preferably at least 800 consecutive nucleotides in length and most preferably at least 900 consecutive nucleotides in length, the consecutive nucleotides being of any one of the nucleic acid sequences given in Table 1. Preferably, the hybridising sequence is one that is capable of hybridising to any of the nucleic acids given in Table 1, or to a portion of any of these sequences, a portion being as defined above. Most preferably, the hybridising sequence is capable of hybridising to a nucleic acid as represented by SEQ ID NO: 1 or to a portion thereof. Preferably, the hybridising sequence encodes an amino acid sequence comprising any one or more of Motifs I to III as defined herein. Preferably, the hybridising sequence encodes an amino acid which when used in the construction of a NAC phylogenetic tree, such as the one depicted in FIG. 1, tends to cluster with the group of NACs comprising the amino acid sequence represented by SEQ ID NO: 2 rather than with any other NAC group.

According to the present invention, there is provided a method for modulating carbon partitioning in plants, comprising introducing and expressing in a plant a nucleic acid capable of hybridizing to any one of the nucleic acids given in Table 1, or comprising introducing and expressing in a plant a nucleic acid capable of hybridising to a nucleic acid encoding an orthologue, paralogue or homologue of any of the nucleic acid sequences given in Table 1.

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tₘ is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tₘ decreases about 1° C. per % base mismatch. The Tₘ may be calculated using the following equations, depending on the types of hybrids:

DNA-DNA hybrids (Meinkoth and Wahl, Anal. Biochem., 138: 267-284, 1984): Tₘ=81.5° C.+16.6×log [Na⁺]^{a}+0.41×%[MG/C^{b}−500×[L^{c}]⁻¹−0.61×% formamide

DNA-RNA or RNA-RNA hybrids: Tₘ=79.8+18.5 (log₁₀[Na⁺]^{a})+0.58 (% G/C^{b})+11.8 (% G/C^{b})²−820/L^{c}

oligo-DNA or oligo-RNA^{d} hybrids:

For <20 nucleotides: Tₘ=2 (Iₙ)

For 20-35 nucleotides: Tₘ=22+1.46 (Iₙ)

^{a} or for other monovalent cation, but only accurate in the 0.01-0.4 M range.

^{b} only accurate for % GC in the 30% to 75% range.

^{c}L=length of duplex in base pairs.

^{d} Oligo, oligonucleotide; Iₙ, effective length of primer=2×(no. of G/C)+(no. of A/T).

Non-specific binding may be controlled using any one of a number of known techniques such as, for example, blocking the membrane with protein-containing solutions, additions of heterologous RNA, DNA, and SDS to the hybridisation buffer, and treatment with Rnase. For non-homologous probes, a series of hybridizations may be performed by one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisations and washes may additionally include 5× Denhardt's reagent, 0.5-1.0% SDS, 100 μg/ml denatured, fragmented salmon sperm DNA, 0.5% sodium pyrophosphate.

For the purposes of defining the level of stringency, reference can conveniently be made to Sambrook et al. (2001) Molecular Cloning: a laboratory manual, 3^{rd} Edition Cold Spring Harbor Laboratory Press, CSH, New York, or to Current Protocols in Molecular Biology, John Wiley & Sons, N.Y. (1989 and yearly updates).

Another nucleic acid variant useful in the methods of the invention is a splice variant encoding a NAC transcription factor as defined hereinabove. The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for making such splice variants are well known in the art.

According to the present invention, there is provided a method for modulating carbon partitioning in plants, comprising introducing and expressing in a plant a splice variant of any one of the nucleic acid sequences given in Table 1, or a splice variant of a nucleic acid encoding an orthologue, paralogue or homologue of any of the amino acid sequences given in Table 1.

Preferred splice variants are splice variants of a nucleic acid represented by SEQ ID NO: 1 or a splice variant encoding an orthologue or paralogue of SEQ ID NO: 2. Preferably, the amino acid encoded by the splice variant comprises any one or more of Motifs I to III as defined herein. Preferably, the amino acid encoded by the splice variant, when used in the construction of a NAC phylogenetic tree, such as the one depicted in FIG. 1, tends to cluster with the group of NACs comprising the amino acid sequence represented by SEQ ID NO: 2 rather than with any other NAC group.

Another nucleic acid variant useful in performing the methods of the invention is an allelic variant of a nucleic acid encoding a NAC transcription factor as defined hereinabove. Allelic variants exist in nature, and encompassed within the methods of the present invention is the use of these natural alleles. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

According to the present invention, there is provided a method for modulating carbon partitioning in plants, comprising introducing and expressing in a plant an allelic variant of any one of the nucleic acids given in Table 1, or comprising introducing and expressing in a plant an allelic variant of a nucleic acid encoding an orthologue, paralogue or homologue of any of the amino acid sequences given in Table 1.

Preferably, the allelic variant is an allelic variant of SEQ ID NO: 1 or an allelic variant of a nucleic acid encoding an orthologue or paralogue of SEQ ID NO: 2. Preferably, the amino acid encoded by the allelic variant comprises any one or more of Motifs I to III as defined herein. Preferably, the amino acid encoded by the allelic variant, when used in the construction of a NAC phylogenetic tree, such as the one depicted in FIG. 1, tends to cluster with the group of NACs comprising the amino acid sequence represented by SEQ ID NO: 2 rather than with any other NAC group.

A further nucleic acid variant useful in the methods of the invention is a nucleic acid variant obtained by gene shuffling. Gene shuffling or directed evolution may also be used to generate variants of nucleic acids encoding NAC transcription factors as defined above. This consists of iterations of DNA shuffling followed by appropriate screening and/or selection to generate variants of nucleic acids or portions thereof encoding NAC transcription factors as defined above having a modified biological activity (Castle et al., (2004) Science 304(5674): 1151-4; U.S. Pat. Nos. 5,811,238 and 6,395,547).

According to the present invention, there is provided a method for modulating carbon partitioning in plants, comprising introducing and expressing in a plant a variant of any one of the nucleic acid sequences given in Table 1, or comprising introducing and expressing in a plant a variant of a nucleic acid encoding an orthologue, paralogue or homologue of any of the amino acid sequences given in Table 1, which variant nucleic acid is obtained by gene shuffling.

Preferably, the variant nucleic acid obtained by gene shuffling encodes an amino acid comprising any one or more of Motifs I to III as defined herein. Preferably, the amino acid encoded by the variant nucleic acid obtained by gene shuffling, when used in the construction of a NAC phylogenetic tree such as the one depicted in FIG. 1, tends to cluster with the group of NACs comprising the amino acid sequence represented by SEQ ID NO: 2 rather than with any other NAC group.

Furthermore, nucleic acid variants may also be obtained by site-directed mutagenesis. Several methods are available to achieve site-directed mutagenesis, the most common being PCR based methods (current protocols in molecular biology. Wiley Eds.

Also useful in the methods of the invention are nucleic acids encoding homologues of any one of the amino acid sequences given in Table 1. “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag·100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break a-helical structures or β-sheet structures). Amino acid substitutions are typically of single residues, but may be clustered depending upon functional constraints placed upon the polypeptide; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company and Table 2 below).

TABLE 2
Examples of conserved amino acid substitutions

| Residue | Conservative Substitutions | Residue | Conservative Substitutions |
|---|---|---|---|
| Ala | Ser | Leu | Ile; Val |
| Arg | Lys | Lys | Arg; Gln |
| Asn | Gln; His | Met | Leu; Ile |
| Asp | Glu | Phe | Met; Leu; Tyr |
| Gln | Asn | Ser | Thr; Gly |
| Cys | Ser | Thr | Ser; Val |
| Glu | Asp | Trp | Tyr |
| Gly | Pro | Tyr | Trp; Phe |
| His | Asn; Gln | Val | Ile; Leu |
| Ile | Leu, Val | | |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, OH), QuickChange Site Directed mutagenesis (Stratagene, San Diego, Calif.), PCR-mediated site-directed mutagenesis or other site-directed mutagenesis protocols.

Also useful in the methods of the invention are nucleic acids encoding derivatives of any one of the amino acids given in Table 1 or orthologues or paralogues of any of the aforementioned SEQ ID NOs. “Derivatives” include peptides, oligopeptides, polypeptides which may, compared to the amino acid sequence of the naturally-occurring form of the protein, such as the one presented in SEQ ID NO: 2, comprise substitutions of amino acids with non-naturally occurring amino acid residues, or additions of non-naturally occurring amino acid residues. Derivatives of the amino acids given in Table 1 are further examples which may be suitable for use in the methods of the invention

“Derivatives” of a protein also encompass peptides, oligopeptides, polypeptides which may comprise naturally occurring altered (glycosylated, acylated, ubiquinated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein.

Furthermore, NAC transcription factors (at least in their native form) typically have DNA-binding activity and an activation domain. A person skilled in the art may easily determine the presence of an activation domain and DNA-binding activity using routine tools and techniques.

Nucleic acids encoding NAC transcription factors may be derived from any natural or artificial source. The nucleic acid may be modified from its native form in composition and/or genomic environment through deliberate human manipulation. Preferably the NAC transcription factor-encoding nucleic acid is from a plant, further preferably from a monocot, more preferably from the Poaceae family, most preferably the nucleic acid is from Oryza sativa.

Any reference herein to a NAC transcription factor is therefore taken to mean a NAC transcription factor as defined above. Any nucleic acid encoding such a NAC transcription factor is suitable for use in performing the methods of the invention.

The present invention also encompasses plants or parts thereof (including seeds) obtainable by the methods according to the present invention. The plants or parts thereof comprise a nucleic acid transgene encoding a NAC transcription factor as defined above.

The invention also provides genetic constructs and vectors to facilitate introduction and/or expression of the nucleic acid sequences useful in the methods according to the invention, in a plant.

Therefore, there is provided a gene construct comprising:
i. Any nucleic acid encoding a NAC transcription factor as defined hereinabove;
ii. One or more control sequences operably liked to the nucleic acid of (i).

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. The invention therefore provides use of a gene construct as defined hereinabove in the methods of the invention.

Plants are transformed with a vector comprising the sequence of interest (i.e., a nucleic acid encoding a NAC transcription factor). The skilled artisan is well aware of the genetic elements that must be present in the vector in order to successfully transform, select and propagate host cells containing the sequence of interest. The sequence of interest is operably linked to one or more control sequences (at least to a promoter). The terms “regulatory element”, “control sequence” and “promoter” are all used interchangeably herein and are to be taken in a broad context to refer to regulatory nucleic acid sequences capable of effecting expression of the sequences to which they are ligated. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ. The term “operably linked” as used herein refers to a functional linkage between the promoter sequence and the gene of interest, such that the promoter sequence is able to initiate transcription of the gene of interest.

Advantageously, any type of promoter, whether natural or synthetic, may be used to drive expression of the nucleic acid sequence. The promoter may be an inducible promoter, i.e. having induced or increased transcription initiation in response to a developmental, chemical, environmental or physical stimulus. An example of an inducible promoter being a stress-inducible promoter, i.e. a promoter activated when a plant is exposed to various stress conditions. The promoter may be a tissue-specific promoter, i.e. one that is capable of preferentially initiating transcription in certain tissues, such as the leaves, roots, seed tissue etc. The term “tissue-specific” as defined herein refers to a promoter that is expressed predominantly in at least one plant tissue or organ, but which may have residual expression elsewhere in the plant due to leaky promoter expression.

According to one preferred feature of the invention, the nucleic acid encoding a NAC-type transcription factor is operably linked to a constitutive promoter. A constitutive promoter is transcriptionally active during most but not necessarily all phases of its growth and development and is substantially ubiquitously expressed. The constitutive promoter is preferably a ubiquitin promoter, more preferably the constitutive promoter is a rice ubiquitin promoter, further preferably the constitutive promoter is represented by a nucleic acid sequence substantially similar to SEQ ID NO: 39, most preferably the constitutive promoter is as represented by SEQ ID NO: 39.

It should be clear that the applicability of the present invention is not restricted to the NAC transcription factor-encoding nucleic acid represented by SEQ ID NO: 1, nor is the applicability of the invention restricted to expression of a such a NAC transcription factor-encoding nucleic acid when driven by an ubiquitin promoter. Examples of other constitutive promoters which may also be used to perform the methods of the invention are shown in Table 3 below.

TABLE 3
Examples of constitutive promoters

| Gene | Expression | |
|---|---|---|
| Source | Pattern | Reference |
| Actin | Constitutive | McElroy et al., Plant Cell, 2: 163-171, 1990 |
| CAMV 35S | Constitutive | Odell et al., Nature, 313: 810-812, 1985 |
| CaMV 19S | Constitutive | Nilsson et al., Physiol. Plant. 100: 456-462, |
| | | 1997 |
| GOS2 | Constitutive | de Pater et al., Plant J Nov; 2(6): 837-44, 1992 |
| Ubiquitin | Constitutive | Christensen et al., Plant Mol. Biol. 18: 675- |
| | | 689, 1992 |
| Rice | Constitutive | Buchholz et al., Plant Mol Biol. 25(5): 837-43, |
| cyclophilin | | 1994 |
| Maize H3 | Constitutive | Lepetit et al., Mol. Gen. Genet. 231: 276-285, |
| histone | | 1992 |
| Actin 2 | Constitutive | An et al., Plant J. 10(1); 107-121, 1996 |

Optionally, one or more terminator sequences (also a control sequence) may be used in the construct introduced into a plant. The term “terminator” encompasses a control sequence which is a DNA sequence at the end of a transcriptional unit which signals 3′ processing and polyadenylation of a primary transcript and termination of transcription. Additional regulatory elements may include transcriptional as well as translational enhancers. Those skilled in the art will be aware of terminator and enhancer sequences that may be suitable for use in performing the invention. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

The genetic construct may optionally comprise a selectable marker gene. As used herein, the term “selectable marker gene” includes any gene that confers a phenotype on a cell in which it is expressed to facilitate the identification and/or selection of cells that are transfected or transformed with a nucleic acid construct of the invention. Suitable markers may be selected from markers that confer antibiotic or herbicide resistance, that introduce a new metabolic trait or that allow visual selection. Examples of selectable marker genes include genes conferring resistance to antibiotics (such as nptll that phosphorylates neomycin and kanamycin, or hpt, phosphorylating hygromycin), to herbicides (for example bar which provides resistance to Basta; aroA or gox providing resistance against glyphosate), or genes that provide a metabolic trait (such as manA that allows plants to use mannose as sole carbon source). Expression of visual marker genes results in the formation of colour (for example β-glucuronidase, GUS), luminescence (such as luciferase) or fluorescence (Green Fluorescent Protein, GFP, and derivatives thereof).

The invention also provides a method for the production of transgenic plants having modulated carbon partitioning relative to control plants, comprising introduction and expression in a plant of any nucleic acid encoding a NAC transcription factor as defined hereinabove.

More specifically, the present invention provides a method for the production of transgenic plants having modulated carbon partitioning, which method comprises:
i. introducing and expressing a nucleic acid encoding a NAC transcription factor (as defined herein) in a plant cell; and
ii. cultivating the plant cell under conditions promoting plant growth and development; and optionally
iii. selecting for plants having modulated carbon partitioning relative to control plants.

The nucleic acid may be introduced directly into a plant cell or into the plant itself (including introduction into a tissue, organ or any other part of a plant). According to a preferred feature of the present invention, the nucleic acid is preferably introduced into a plant by transformation.

The term “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated from there. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

Transformation of plant species is now a fairly routine technique. Advantageously, any of several transformation methods may be used to introduce the gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al. (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al. (1986) Mol. Gen Genet 202: 179-185); DNA or RNA-coated particle bombardment (Klein T M et al. (1987) Nature 327: 70) infection with (non-integrative) viruses and the like. Transgenic rice plants are preferably produced via Agrobacterium-mediated transformation using any of the well known methods for rice transformation, such as described in any of the following: published European patent application EP 1198985 Al, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth.

Generally after transformation, plant cells or cell groupings are selected for the presence of one or more markers which are encoded by plant-expressible genes co-transferred with the gene of interest, following which the transformed material is regenerated into a whole plant.

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, or quantitative PCR, all techniques being well known to persons having ordinary skill in the art.

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a NAC transcription factor as defined hereinabove. Preferred host cells according to the invention are plant cells.

The invention also extends to harvestable parts of a plant such as, but not limited to seeds, leaves, fruits, flowers, stems, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression. For example, endogenous promoters may be altered in vivo by mutation, deletion, and/or substitution (see, Kmiec, U.S. Pat. No. 5,565,350; Zarling et al., PCT/US93/03868), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

Other control sequences (besides promoter, enhancer, silencer, intron sequences, 3′UTR and/or 5′UTR regions) may be protein and/or RNA stabilizing elements.

As mentioned above, a preferred method for modulating (preferably, increasing) expression of a nucleic acid encoding a NAC transcription factor is by introducing and expressing in a plant a nucleic acid encoding a NAC transcription factor; however the effects of performing the method, i.e. modulating carbon partitioning in plants may also be achieved using other well known techniques. A description of some of these techniques will now follow.

One such technique is T-DNA activation tagging (Hayashi et al. Science (1992) 1350-1353), which involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

The effects of the invention may also be reproduced using the technique of TILLING (Targeted Induced Local Lesions In Genomes). This is a mutagenesis technology useful to generate and/or identify a nucleic acid encoding a NAC transcription factor with modified expression and/or activity. TILLING also allows selection of plants carrying such mutant variants. These mutant variants may exhibit modified expression, either in strength or in location or in timing (if the mutations affect the promoter for example). These mutant variants may exhibit higher NAC transcription factor activity than that exhibited by the gene in its natural form. TILLNG combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet 5(2): 145-50).

The effects of the invention may also be reproduced using homologous recombination, which allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offringa et al. (1990) EMBO J 9(10): 3077-84) but also for crop plants, for example rice (Terada et al. (2002) Nat Biotech 20(10): 1030-4; lida and Terada (2004) Curr Opin Biotech 15(2): 132-8).

Plants having modulated carbon partitioning may exhibit altered architecture. Altered plant architecture may be manifested by one or more of: altered number of tillers (culms); altered diameter of tillers; altered plant size; altered number of panicles; altered panicle length; altered seed size; altered leaf width.

Additionally or alternatively, plants having modulated carbon partitioning may exhibit altered photosynthesis.

Additionally or alternatively, plants having modulated carbon partitioning may have an altered starch and/or sugar content. The altered starch and/or sugar content may be a change in the quantity and/or quality and/or composition of the starch and/or sugar. The altered starch and/or sugar content may result from any one or more of: altered sucrose synthase activity, altered sucrose phosphate synthase activity, altered invertase activity, altered beta amylase activity, altered carbohydrate levels, altered sugar flux, altered degradation of sugars and altered auxin flux. The sugar is preferably one or more of glucose, sucrose, fructose, arabinose and galactose, but could be any other sugar.

Additionally or alternatively, plants having modulated carbon partitioning may display altered lignin accumulation.

Preferably, plants having increased expression of a NAC transcription factor display one or more of the following: increased number of tillers (culms); reduced diameter of tillers; increased fresh weight; smaller plants; increased number of panicles; increased panicle length; increased seed weight per plant; increased seed size; increased Thousand Kernel Weight (TKW); increased grain filling; reduced width of flag leaves; changed vascular bundles in flag leaves (decrease in the number of large vascular bundles and an increase in the number of small vascular bundles); change in number of veins; thinner leaves; increased number of stomata; changed sugar content, composition, quality; increased sucrose synthase activity (both at heading and ripening), increased sucrose phosphate synthase activity (especially at heading time); increased invertase in flag leaves (especially at ripening time); reduced beta amylase activity; increased starch in leaves (at ripening and heading); increased glucose, sucrose, fructose (at heading time); increased arabinose and galactose (at ripening); increased vasculature and stomatal density; change in auxin flux/sugar flux; increased lignin accumulation; Delayed senescence with delayed degradation of sugars.

Preferably, plants having decreased or substantially eliminated expression of a NAC transcription factor display one or more of the following: reduced number of tillers (culms), increased diameter of tillers, bigger tillers, reduced number of panicles, reduced panicle length, reduced seed weight per plant, increased seed size, reduced grain filling or fertility (% fertile seeds per panicle), increased width of flag leaves, thinner leaves, altered vascular bundles with an increase in the number of large vascular bundles and a decrease in small vascular bundles, altered number of leaf veins, decreased number of stomata, reduced photosynthesis, decreased fresh weight, reduced sucrose synthase activity (both at heading and ripening), reduced sucrose phosphate synthase activity (at heading and ripening), decreased invertase in flag leaves (both at heading and ripening), Beta amylase activity increased (especially at heading time), decreased starch in flag leaves (especially at heading), decreased sucrose, increased glucose, increase in fructose at ripening, decreased arabinose (particularly at senescence), decreased vasculature and stomatal density, Change in auxin flux/sugar flux, less lignin accumulation, accelerated senescence with faster degradation of sugars and less transferred to the sink (more efficient sink production).

The methods of the invention are advantageously applicable to any plant. The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Diheteropogon amplectens, Dioclea spp, Dolichos spp., Dorycnium rectum, Echinochloa pyramidalis, Ehrartia spp., Eleusine coracana, Eragrestis spp., Erythrina spp., Eucalyptus spp., Euclea schimperi, Eulalia villosa, Fagopyrum spp., Feijoa sellowiana, Fragaria spp., Flemingia spp, Freycinetia banksii, Geranium thunbergii, Ginkgo biloba, Glycine javanica, Gliricidia spp, Gossypium hirsutum, Grevillea spp., Guibourtia coleosperma, Hedysarum spp., Hemarthia altissima, Heteropogon contortus, Hordeum vulgare, Hyparrhenia rufa, Hypericum erectum, Hyperthelia dissoluta, Indigo incarnata, Iris spp., Leptarrhena pyrolifolia, Lespediza spp., Lettuca spp., Leucaena leucocephala, Loudetia simplex, Lotonus bainesii, Lotus spp., Macrotyloma axillare, Malus spp., Manihot esculenta, Medicago sativa, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthria fleckii, Pogonarthria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys verticillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugarcane, sunflower, tomato, squash, tea and algae, amongst others.

According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, sorghum and oats.

The present invention also encompasses use of nucleic acids encoding the particular type of NAC transcription factors described herein and use of these NAC transcription factors in modulating carbon partitioning in plants.

Nucleic acids encoding the particular type of NAC transcription factors described herein, or the NAC transcription factors themselves, may find use in breeding programmes in which a DNA marker is identified which may be genetically linked to a NAC transcription factor-encoding gene. The nucleic acids/genes, or the NAC transcription factors themselves may be used to define a molecular marker. This DNA or protein marker may then be used in breeding programmes to select plants having modulated carbon partitioning as defined hereinabove in the methods of the invention.

Allelic variants of a NAC transcription factor-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give traits related to modulated carbon partitioning. Selection is typically carried out by monitoring performance of plants containing different allelic variants of the sequence in question. Performance may be monitored in a greenhouse or in the field. Further optional steps include crossing plants in which the superior allelic variant was identified with another plant. This could be used, for example, to make a combination of interesting phenotypic features.

Nucleic acids encoding NAC transcription factors may also be used as probes for genetically and physically mapping the genes that they are a part of, and as markers for traits linked to those genes. Such information may be useful in plant breeding in order to develop lines with desired phenotypes. Such use of NAC transcription factor encoding nucleic acids requires only a nucleic acid sequence of at least 15 nucleotides in length. The NAC transcription factor-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the NAC transcription factor-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the NAC transcription factor-encoding nucleic acid in the genetic map previously obtained using this population (Botstein et al. (1980) Am. J. Hum. Genet. 32:314-331).

The production and use of plant gene-derived probes for use in genetic mapping is described in Bematzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favor use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having modulated carbon partitioning, as described hereinbefore. These traits may also be combined with other economically advantageous traits, such as yield-enhancing traits, tolerance to other abiotic and biotic stresses, traits modifying various architectural features and/or biochemical and/or physiological features.

### DESCRIPTION OF FIGURES

The present invention will now be described with reference to the following figures in which:

FIG. 1 shows a phylogenetic tree taken from Ooka et al., 2003 (DNA Research 10, 239-247). The dashed box 3^{rd} from the bottom, marked “OsNAC7”, is the group comprising the sequence of SEQ ID NO: 2. Amino acid sequences clustering in this group are considered to be useful in the methods of the invention.

FIG. 2 shows a phylogenetic tree showing the position of NAM2 compared to NAM2 homologues SQ23 and SQ9, which homologues have been shown to give comparable phenotypes to NAM2 OE plants (see FIGS. 20, 21 and 23).

FIG. 3 shows an alignment of NAC transcription factors as defined hereinabove. The sequences were aligned using AlignX program from Vector NTI suite (InforMax, Bethesda, Md.). Multiple alignment was done with a gap opening penalty of 10 and a gap extension of 0.01. Minor manual editing was also carried out where necessary to better position some conserved regions. The NAC domain and Motifs Ito III are indicated.

FIG. 4 shows wild type (WT) plants, NAM2 mutant (KO) plants and plants overexpressing (OX) NAM2 at harvest stage.

FIG. 5 shows the stumps of WT, KO and OX plants

FIG. 6 shows Northern blots for NAM2 mRNA in WT, KO and OX lines

FIG. 7 shows the distribution of panicle lengths of WT, KO and OX lines

FIG. 8 shows the diameters of the lowest node of the main culms in WT, KO and OX plants

FIG. 9 shows the width of flag leaves in WT, KO and OX plants

FIG. 10 shows the difference in vein numbers among leaves of WT, KO and OX lines

FIG. 11 shows OX lines having a higher number of stomata in the leaves

FIG. 12 shows the rate of photosynthesis in flag leaves. The stages at which the measurements were made were as follows: I: 7 days before heading (7 days), II: heading, III: 7 days after heading, IV: 14 days after heading, V: 28 days after heading (grains being ripened), VI: 42 days after heading.

FIG. 13 shows the activities of sucrose synthases in flag leaves. The stages at which the measurements were made were as follows: I: 7 days before heading (7 days), II: heading, III: 7 days after heading, IV: 14 days after heading, V: 28 days after heading (grains being ripened), VI: 42 days after heading.

FIG. 14 shows the activities of sucrose phosphate synthases in flag leaves. The stages at which the measurements were made were as follows: I: 7 days before heading (7 days), II: heading, III: 7 days after heading, IV: 14 days after heading, V: 28 days after heading (grains being ripened), VI: 42 days after heading.

FIG. 15 shows the activities of invertases in flag leaves. The stages at which the measurements were made were as follows: I: 7 days before heading (7 days), II: heading, III: 7 days after heading, IV: 14 days after heading, V: 28 days after heading (grains being ripened), VI: 42 days after heading.

FIG. 16 shows the activities of beta-amylases in flag leaves. The stages at which the measurements were made were as follows: I: 7 days before heading (7 days), II: heading, III: 7 days after heading, IV: 14 days after heading, V: 28 days after heading (grains being ripened), VI: 42 days after heading.

FIG. 17 shows levels of sucrose, glucose and fructose in flag leaves of WT, KO and OX plants before ripening stage.

FIG. 18 shows levels of arabinose and galactose in flag leaves of WT, KO and OX plants after the ripening stage.

FIG. 19 shows a photomicrograph of lignin stained with phloroglucinol-HCL (Stage V) in WT, KO and OX plants.

FIG. 20 shows a photo comparing NAM2 and SQ23 (a NAM2 homologue) and SQ9 OX plants and WT plants.

FIG. 21 shows seed yield in WT, SQ23 (NAM2 homologue) and SQ9 (NAM2 homologue) plants.

FIG. 22 shows the stumps of WT, SQ23-OX and SQ9-OX plants

### EXAMPLES

The present invention will now be described with reference to the following examples, which are by way of illustration alone. The following examples are not intended to completely define or otherwise limit the scope of the invention.

### Example 1

### Cloning of OsNAM2 cDNA and Vector Construction

The full length NAM2 cDNA (975 bp) was cloned into HpaI and KpnI sites of pGA1611(Sung et al., 2003 A Complete Sequence of the pGA1611 Binary Vector. J of Plant Biology 46: 211-214) with a ubiquitin promoter and a NOS terminator. T-DNA vectors were transformed into LBA4404. In transforming rice calli with T-DNA carrying hygromycin phosphotransferase (HPT), a published method of tissue culture (Hiei et al., 1994 was followed (Efficient transformation of rice (Oryza sativa L.) mediated by Agrobacterium and sequence analysis of the boundaries of the T-DNA. Plant J 6: 271-282). Transformed rice plants were allowed to grow and were then examined for the parameters described below.

### Example 3

### Phenotypic Measurements

T2 transgenic lines were grown in the field. Plant height and panicle length were measured with a standard ruler. The numbers of tillers, panicles, and grains were manually counted by eye. The weights of total grains in plants and 1000 kernels were measured using a weighing scale.

### Example 4

### Enzyme and Biochemical Measurements

A. Measurements of Enzyme Activities

The topmost upper leaves were used to measure enzyme activities.

5.1 Invertase

Invertase activity was measured as described below and in Ishimaru et al., Expression patterns of genes encoding carbohydrate-metabolizing enzymes and their relationship to grain filling in rice. Plant Cell Physiology 46:620-628, 2005.
(1) a leaf disc (0.5 cm²) was homogenized in the extraction buffer (1 ml)
(2) extracts were cfg. at 15,000 rpm for 5 min at 4° C.
(3) pellets were washed twice with 5 mM Tris-Hcl (pH 7.0)
(4) samples were cgf at 15.000 rpm for 10 min at 4° C.
(5) pellets were suspended in 1 ml 50 mM sodium acetate buffer (pH 5.2) containing 0.1M sucrose

(6) the mixture was incubated at 37° C. for 90-120 min

(7) the mixture was neutralized by adding an aliquot of 1M Tris-HCl pH 8.0, and subsequently the reaction was stopped by heat inactivation at 95° C. for 5 min, and samples went through filters(pore size, 0.45 um)

(8) the amount of glucose formed was measured with the assay kit (Sigma cat #GAHK20)

Extraction Buffer

50 mM Tris, 5 mM MgCl₂, 5 mM DTT, 1 mM EDTA, 1 mM EGTA, 15% glycerine, 01. mM PMSF

5.2 Sucrose-Synthase

Sucrose synthase activity was measured as described below and in Ishimaru et al., Expression patterns of genes encoding carbohydrate-metabolizing enzymes and their relationship to grain filling in rice. Plant Cell Physiology 46:620-628, 2005.
(1) leaf samples (equivalent to 50 mg dry weight) were powdered in liquid nitrogen
(2) powders were suspended in an extraction buffer (1 ml).
(3) homogenate was centrifuged at 12,000×g for 10 min, and supernatants was used for the assays of sucrose-synthse activity. This step was conducted at 0-4° C.
(4) the reaction (total 350 ul) was started by adding 10 μl of sample in the assay mixture containing 55 mM HEPES-NaOH (pH 7.4), 16 mM MgCl₂, 8 mM fructose and 8 mM UDP-glucose.
(5) the reaction was incubated at 30° C. for 20 min
(6) the reaction was stopped by adding 1M NaOH
(7) after being cooled, 0.25 ml of 0.1% (w/v) resorcinol in 95% ethanol and 0.75 ml of 30% HCl were added, and the mixture was incubated at 80° C. for 8 min.
(8) tubes were cooled to the room temperature and the A₅₂₀ was measured.

Extraction Buffer

100 mM HEPES-NaOH (pH 7.4), 8 mM MgCl₂, 2 mM K₂HPO₄, 2 mM EDTA, 12.5% (v/v) glycerol and 50 mM 2-mercaptoethanol.

5.3 Sucrose Phosphate Synthase

Sucrose phosphate synthase activity was measured as described below and in Kerr et al. Effect of N-source on soybean leaf sucrose phosphate synthase, starch formation, and whole plant growth. Plant Physiology 75: 483-488, 1984.
(1) sample (50-100 mg) were ground in 8 vols (400-800 ul) of the ice-cold extraction buffer
(2) the homogenate was centrifuged at 18,500×g for 10 min, and the supernatant was used for the assays. This step was conducted at 0-4° C.
(3) 20 ul supernatants were added to the assay mixture (100 ul) contained 7.5 mM UDPG, 7.5 mM fructose-6-P, 15 mM MgCl₂, 50 mM Hepes-NaOH (pH 7.5).
(4) mixtures were incubated at 25° C. for 10 min
(5) the reaction was terminated by addition of 1.0 N NaOH.
(6) Unreacted fructose-6-P was destroyed by heating the mixtures in a boiling water bath for 10 min.
(7) After cooling, 0.25 ml of 0.1% (w/v) resorcinol in 95% ethanol and 0.75 ml of 30% HCl were added, and the mixture was incubated at 80° C. for 8 min.
(8) The tubes were cooled to room temperature and the A₅₂₀ was measured.

Extraction Buffer

100 mM HEPES-NaOH (pH 7.4), 8 mM MgCl₂, 2 mM K₂HPO₄, 2 mM EDTA, 12.5% (v/v) glycerol and 50 mM 2-mercaptoethanol.)

5.4 β-amylase

The activity was measured following the protocol provided in the beta-amylase assay kit (Megazyme, cat#K-BETA,)
(1) samples (equivalent to 0.5 g dry weight) were powdered in liquid nitrogen
(2) 1.0 ml of extraction buffer (Buffer A) was added
(3) extracts were left over 1 hr period at room temperature, with frequent vigorous stirring an a vortex mixer (5 times over the 1 hour period)
(4) samples went through the filter (0.45 um)
(5) 0.2 ml of filtrate were diluted with 10 ml in buffer B, mix, and then 0.2 ml of this solution was further diluted to 5 ml in Buffer B.
(6) 0.2 ml aliquots of Betamyl substrate solution was dispensed into the bottom of glass test tubes and the contents were pre-incubated at 40° C. for approx 5 min
(7) samples was pre-incubate at 40° C. for approx. 5 min
(8) To each tube containing Betamyl substrate solution, 0.2 ml of pre-equilibrated enzyme preparation were added directly to the bottom of the tube, mix, and then incubated at 40° C. for exactly 10 min
(9) At the end of the 10 min incubation period, 3.0 ml of stopping reagent were added and the tube contents were stirred.
(10) the absorbance at 400 nm were read.

| | |
|---|---|
| Extraction buffer (buffer A) | |
| 0.05M Tris-HCl pH 8.0 | 6.06 g/L |
| 1 mM EDTA | 0.37 g/L |

Immediately before use, add 1.75 g cysteine HCl (100 mM) in
100 ml extraction buffer
Adjust pH to 8.0
Stable for 8 hr at 4° C.

| | |
|---|---|
| Dilution buffer (buffer B) | |
| 0.1M maleic acid | 11.6 g/L |
| 1 mM EDTA | 0.37 g/L |
| 1.0 mg/ml BSA | 1.0 g/L |

pH 6.2(with 4M sodium hydroxide 16 g/100 ml)
add 0.2 g of sodium azide
keep on 4° C.

| | |
|---|---|
| Stopping reagent | |
| 1% (w/v) Tris-HCl(pH 8.5) | 10 g/L |

B. Measurements of Carbohydrates

Carbohydrates were measured from the same tissues that were used for enzyme activities. Starches were quantified based on the published protocol using the starch assay kit (Sigma cat#S5296). For measurement of sucrose, glucose, fructose, arabinose, and galactose, pretreated samples were sent to NICEM (The National Instrumentation Center for Environmental Management; http://www.nicem.snu.ac.kr) (Seoul National University in Seoul, South Korea) where carbohydrates were measured by HPLC.

### REFERENCE

Ishimaru K, Hirose T, Aoki N, Takahashi S, Ono K, Yamamoto S, Wu J, Saji S, Baba T, Ugaki M, Matsumoto T, Ohsugi R. (2001) Antisense expression of a rice sucrose transporter OsSUT1 in Rice (Oryza sativa L.). Plant Cell Physiol. 42(10) : 1181-1185

5.5. Starches
(1) samples (equivalent to 0.5 g dry weight) were powdered in liquid nitrogen in a mortar with a pestle and extracted twice with 80% (v/v) ethanol at 80° C.
(2) After centrifugation at 2,000×g for 5 min, the supernatants were collected, dried in a vacuum
(3) the pellets were boiled in distilled water for 2 h
(4) according to the starch assay kit (Sigma cat #S5296), samples were digested with amyloglucosidase for 15 min at 55° C.
(5) the increase in A₃₄₀ were measured

### 5.6 Sucrose, Glucose, Fructose, Arabinose, Galactose Sample Pretreatment

(1) 1 gm leaf tissue was ground in 10 ml dH₂O
(2) samples were shaken at 40 rpm for 4 hours at RT
(3) samples were cfg at 15,000 rpm for 30 min at 4° C.
(4) supernatants went through C18 Sepak (Waters, cat#Sep-pak plus C18) equipped with 0.45 um filters
(5) samples were frozen and send to NICEM (The National Instrumentation Center for Environmental Management)
(6) carbohydrates were measured with HPLC.

HPLC Setting
.Column: Nucelogel® Sugar Ca 300×6.5 mm
.Detector: RID detector
.Flow rate: 0.7 ml/min
.Column temp: 80° C. (Max: 85° C.)
.Sample injection: 10 μg (sucrose, glucose, fructose, mannitol, sorbitol 500 μg solutions)

5.7 lignin Stains

For relative measurement of lignin contents, free-hand cross-sections were stained with phloroglucinol. Flag leaves (stage V; 28 days after heading) were used for lignin stains.
(1) tissues were fixed in 2.5% glutaraldehyde and 0.1M Na-cacodylate(pH. 7.4)
(2) samples were cross-sectioned with a razor by hand (free-hand sections)
(3) samples were stained with 2%(w/v) phloroglucinol in 95% ethanol
(4) Immediately, sections were observed under the light and fluorescence microscopy the Axioplan 2 (Carl Zeiss Co.)

### REFERENCE

Biemelt S, Tschiersch H, Sonnewald U (2004) Impact of altered gibberellin metabolism on biomass accumulation, lignin biosynthesis, and photosynthesis in transgenic tobacco plants. Plant physiology 135(1): 254-265.

Wang H, Hao J, Chen X, Hao Z, Wang X, Lou Y, Peng Y, Guo Z(2007) Overexpression of rice WRKY89 enhances ultraviolet B tolerance and disease resistance in rice plants. Plant Molecular Biology 65(2) : 799-815.

C. Vascular Bundles Measurements,

Pieces of flag leaves (stage V; 28 days after heading) were fixed in the FAA solution and embedded in paraplasts. Cross-sections (10 um) were made using a microtome. Samples were examined under the light microscope (Axioplan 2, Carl Zeiss Co.). No morphological abnormality in vascular bundles was detected in mutants and overexpression lines.

### Example 5

### Results

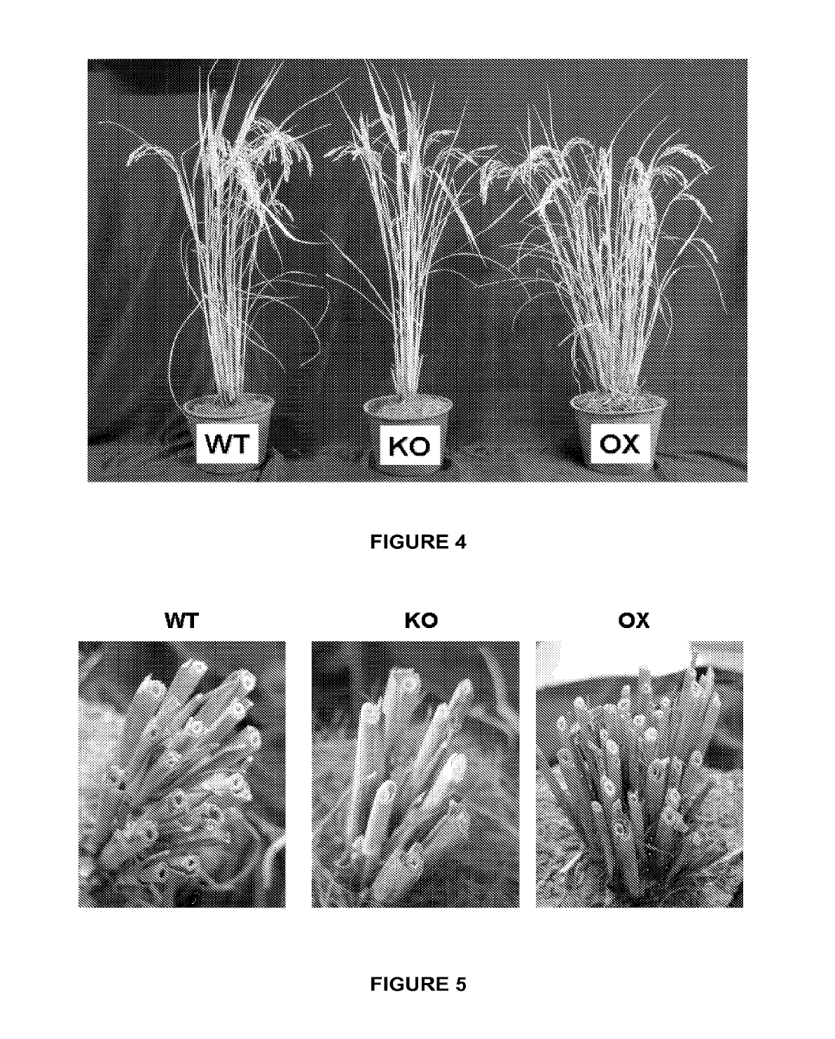

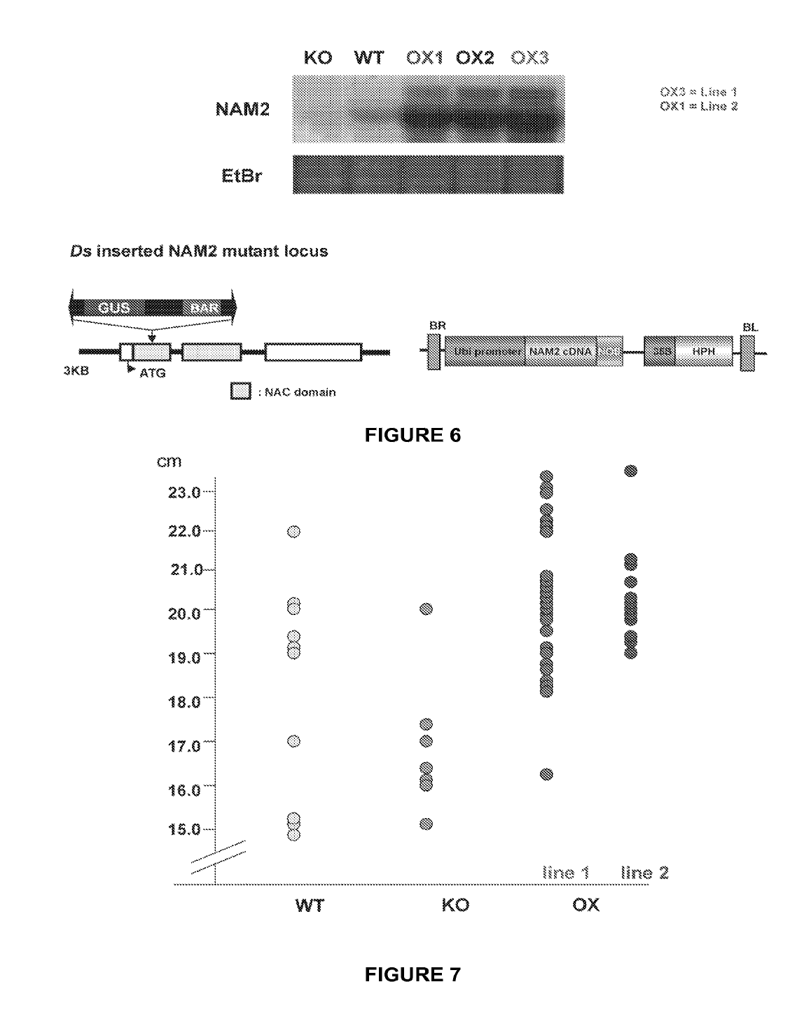

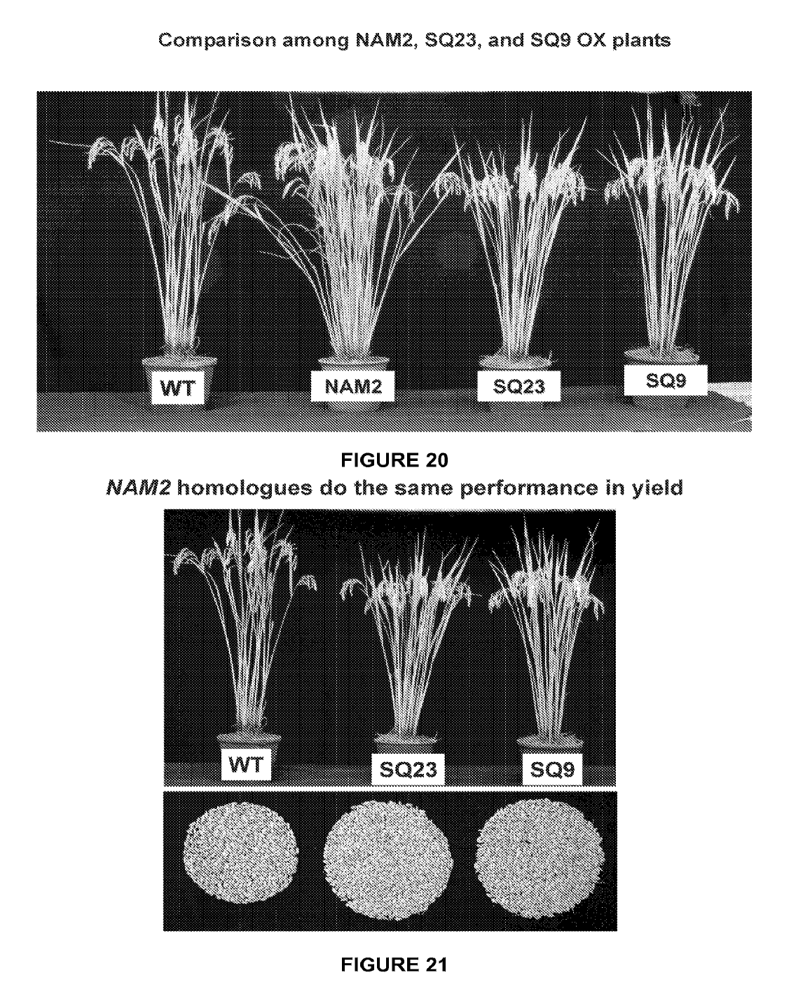

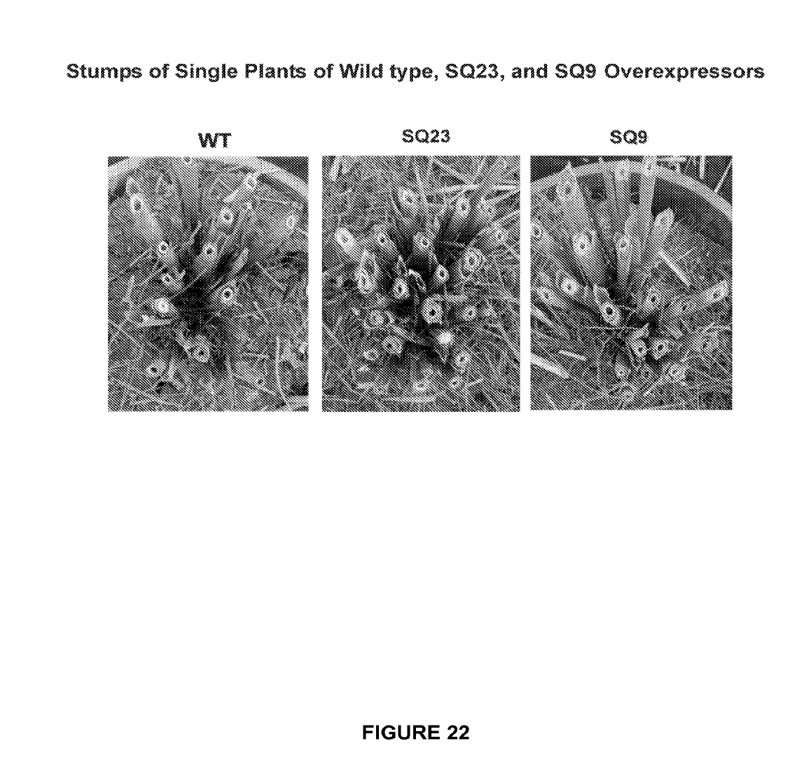

Plants overexpressing NAM2 (OE) at the harvest stage show an increased number of tillers (culms), and an increase in the number and length of panicles, compared to wild type (WT) or knock out (KO) plants. The OE plants are also smaller/shorter compared to the KO and WT plants (see FIGS. 4, 5 and 7). The OE plants have increased fresh weight; increased seed weight per plant; increased seed size; increased Thousand Kernel Weight (TKW); increased grain filling compared to KO and WT plants 9 (see FIGS. 20 and 21).

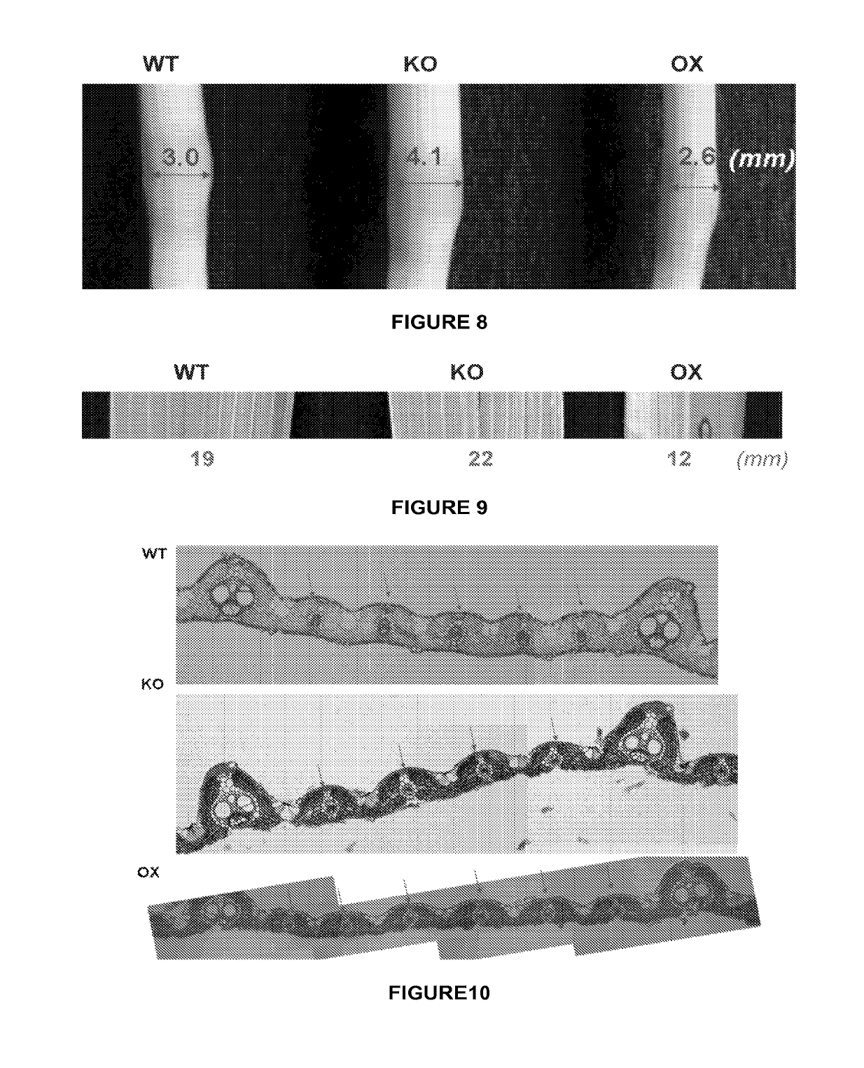

Plants overexpressing NAM2 (OE) show a reduced diameter of tillers (culms) compared to wild type (WT) or knock out (KO) plants (see FIG. 8).

Plants overexpressing NAM2 (OE) show a reduction in the width of flag leaves compared to wild type (WT) or knock out (KO) plants (see FIG. 9).

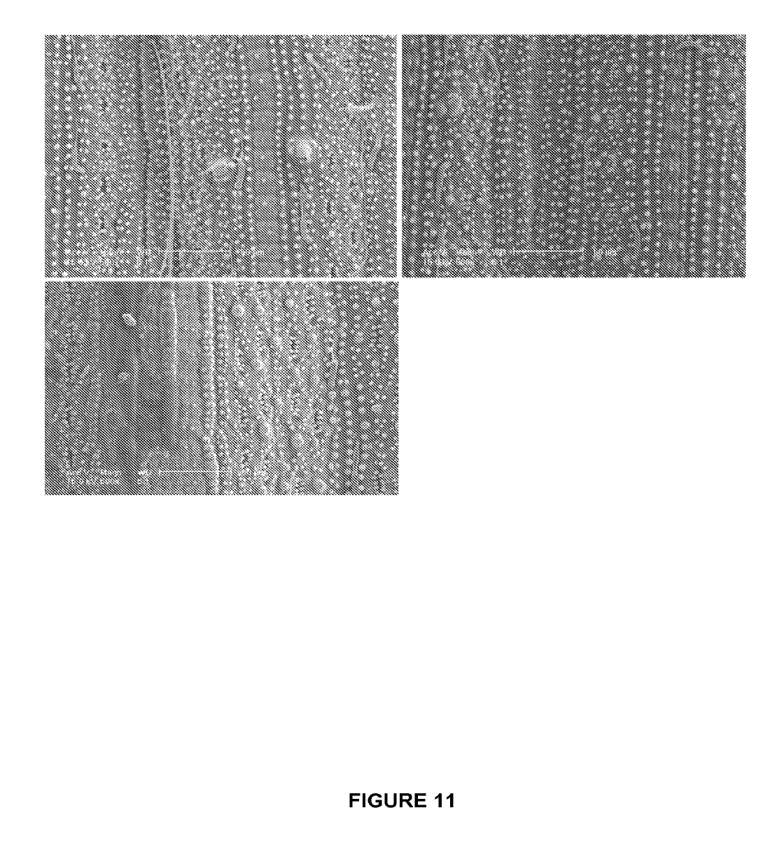

Plants overexpressing NAM2 (OE) also show a decrease in the number of large vascular bundles and an increase in the number of small vascular bundles compared to wild type (WT) or knock out (KO) plants. The change in vasculature could lead to better nutrient and water transport, which in turn could result in increased plant fitness. OE plants also show an increase in the number of veins, thinner leaves and an increase in the number of stomata compared to KO and WT plants (see FIGS. 10 and 11).

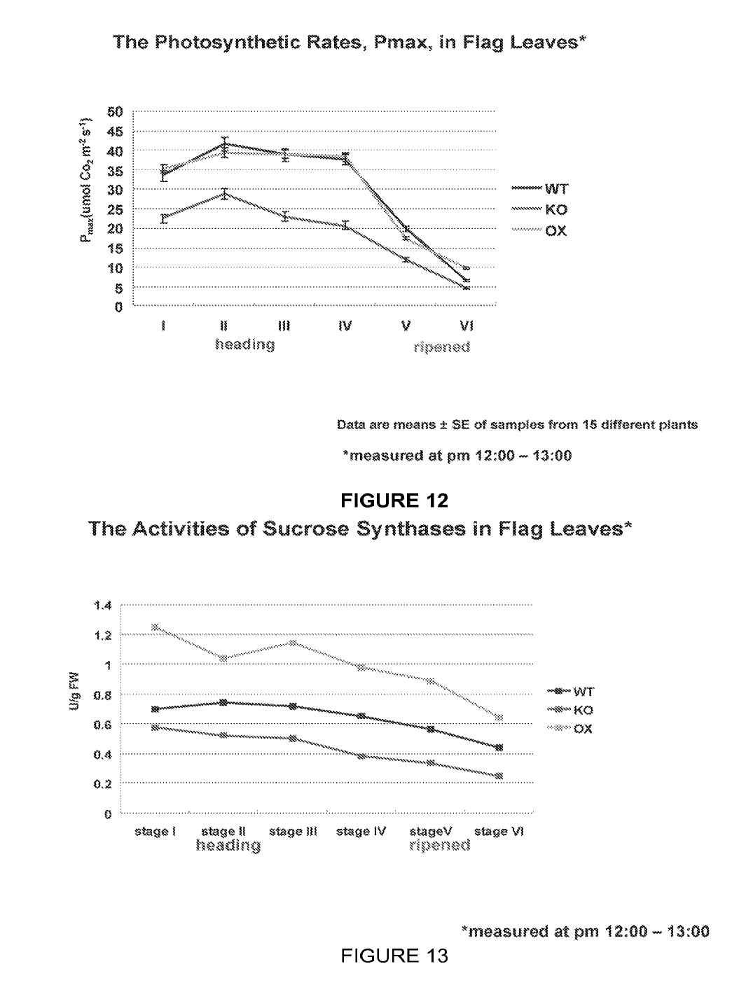

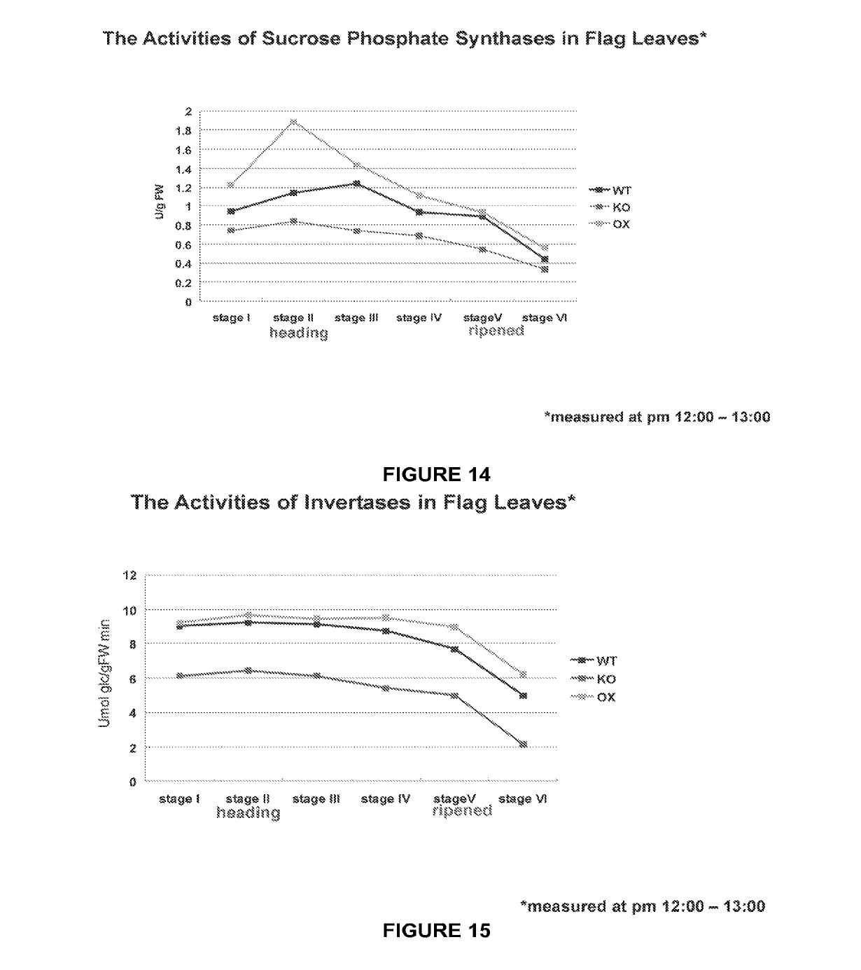

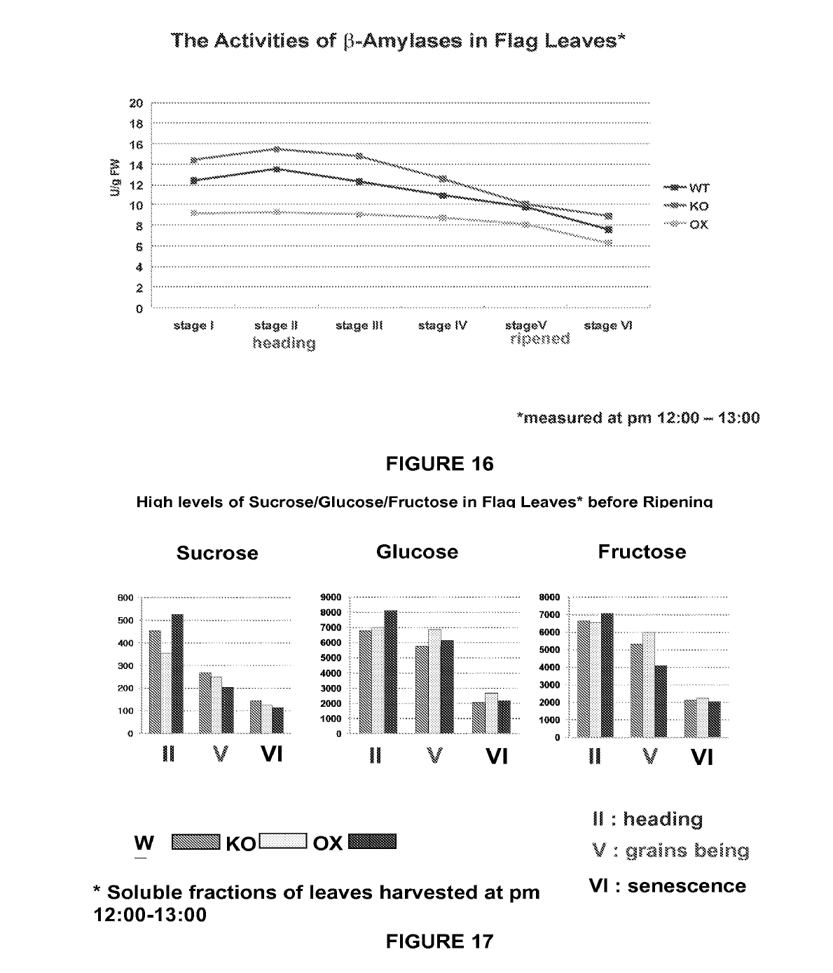

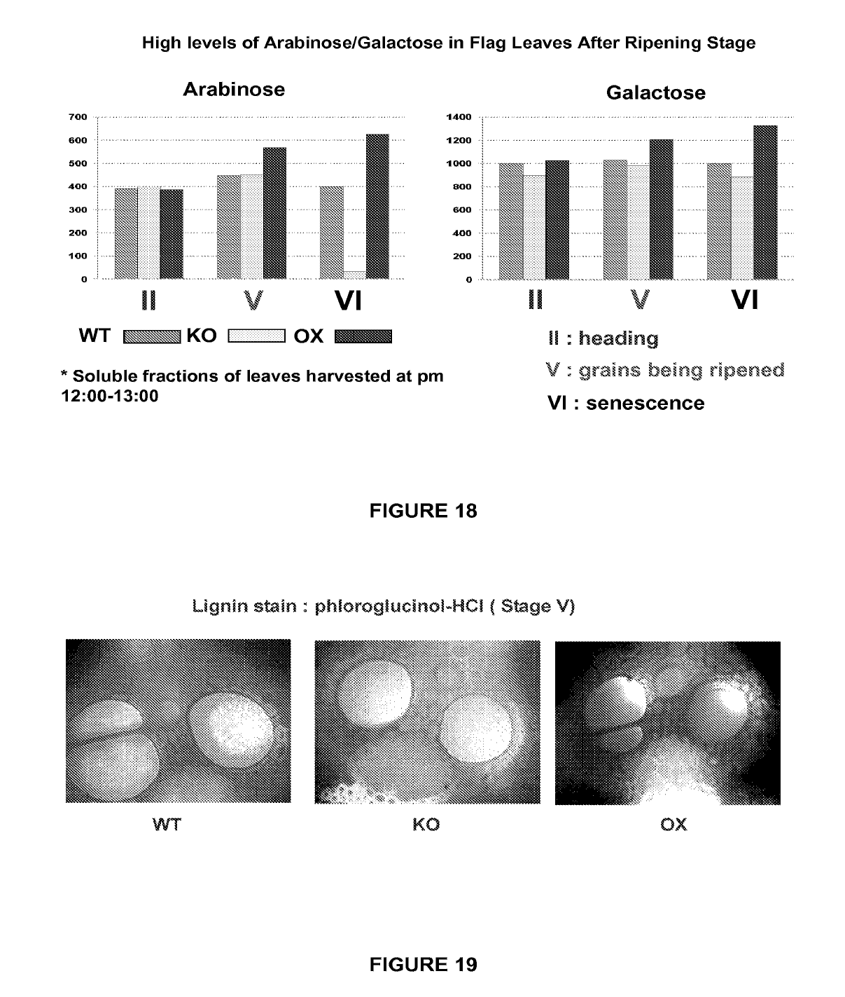

Plants overexpressing NAM2 (OE) also show changes in the following compared to KO and WT plants: increased sucrose synthase activity in flag leaves (both at heading and ripening) (see FIG. 13); increased sucrose phosphate synthase activity (especially at heading time) (see FIG. 14); increased invertase activity in flag leaves (especially at ripening time) (see FIG. 15); reduced beta amylase activity (see FIG. 16); increased starch in leaves (at ripening and heading); increased glucose, sucrose, fructose (at heading time) (see FIG. 17); increased arabinose and galactose (at ripening) (see FIG. 18); increased vasculature and stomatal density (see FIG. 11); change in auxin flux/sugar flux (see FIG. 12); increased lignin accumulation (see FIG. 19); Delayed senescence with delayed degradation of sugars (see FIG. 18).

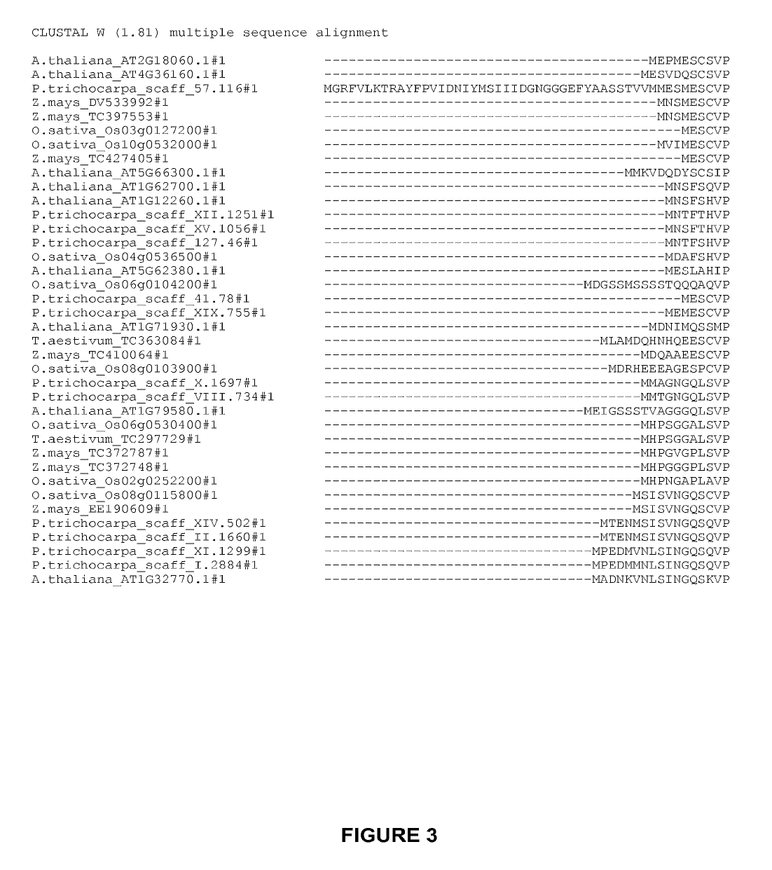

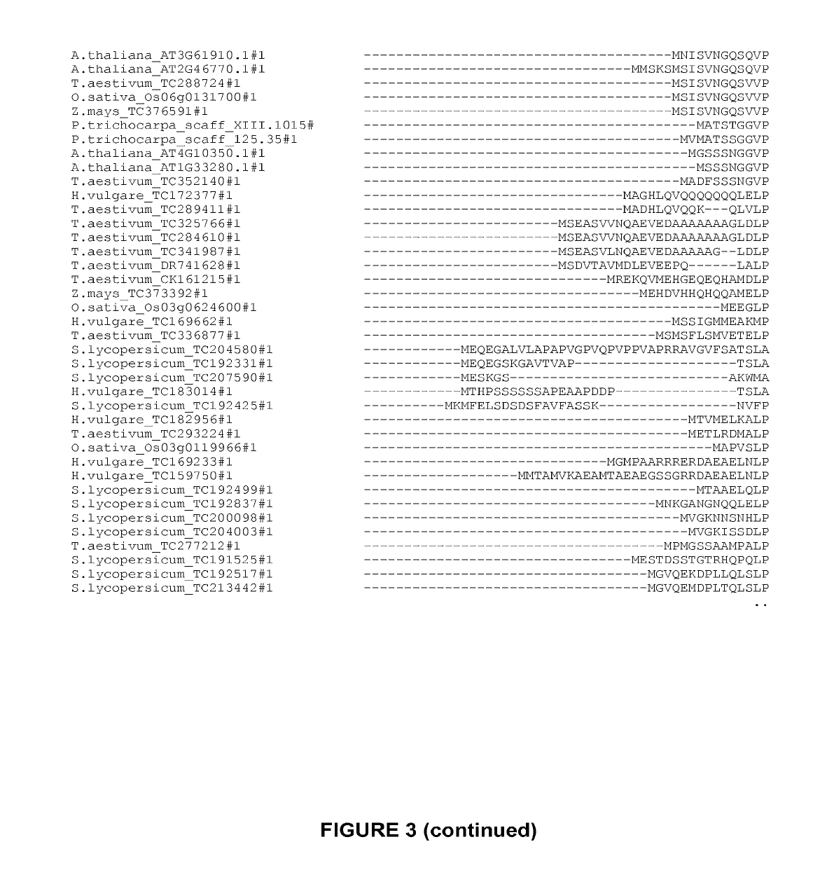

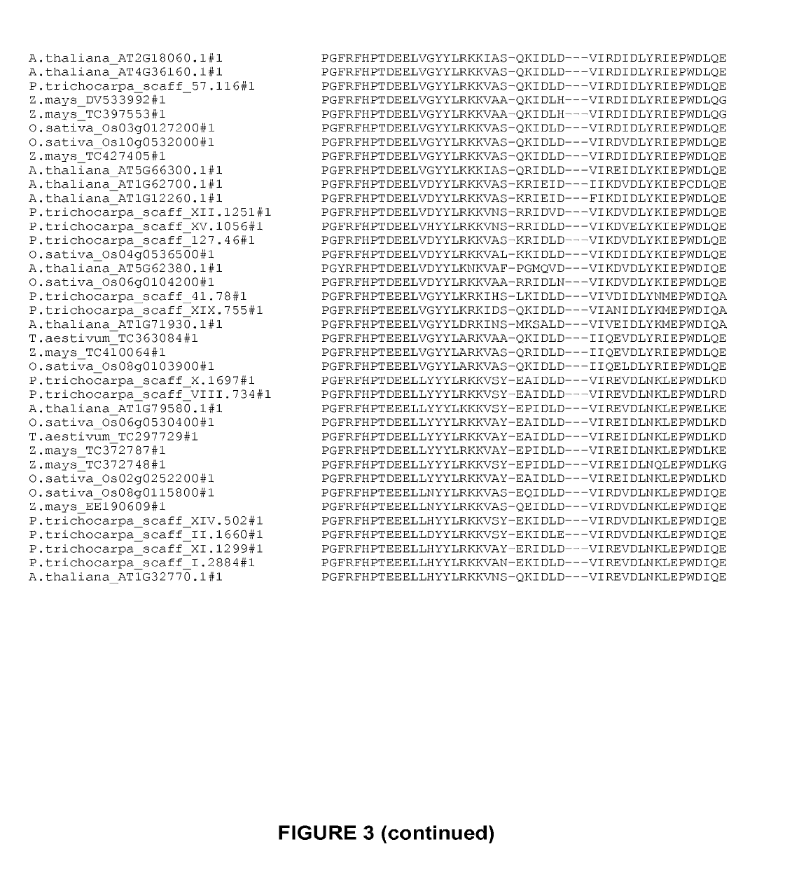

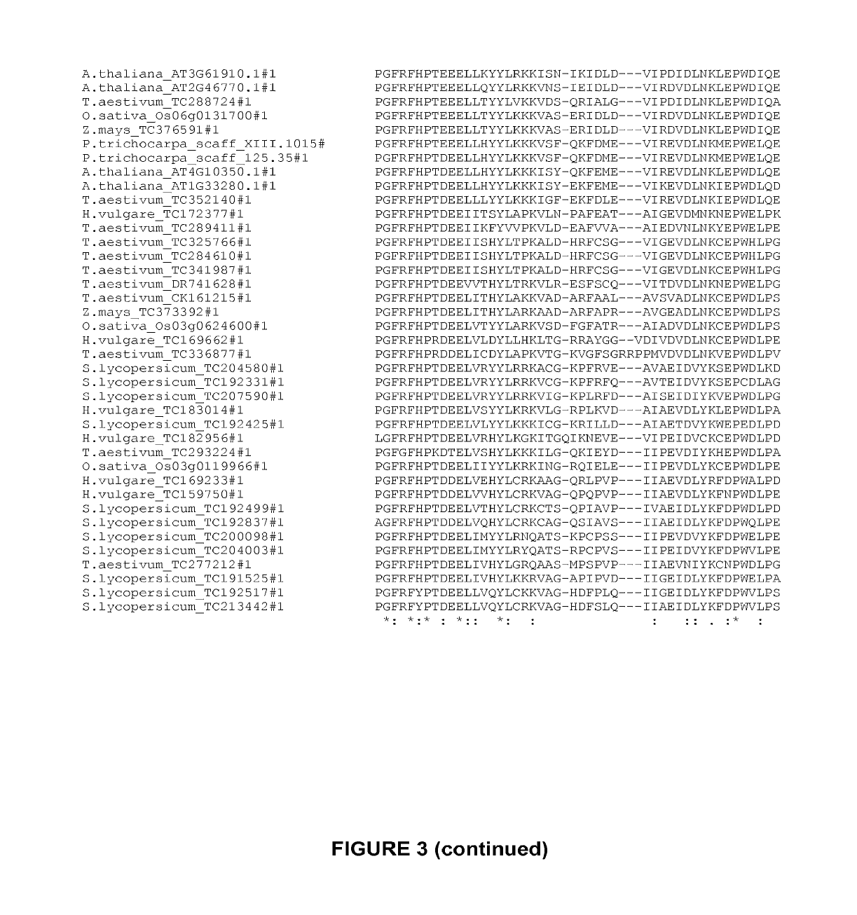

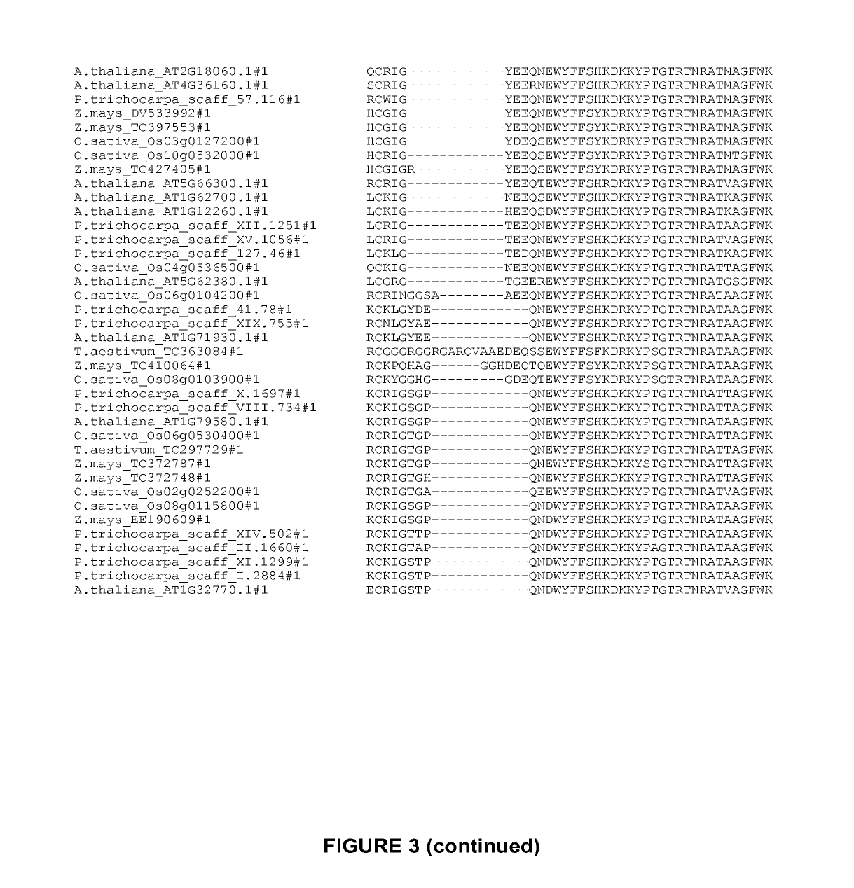

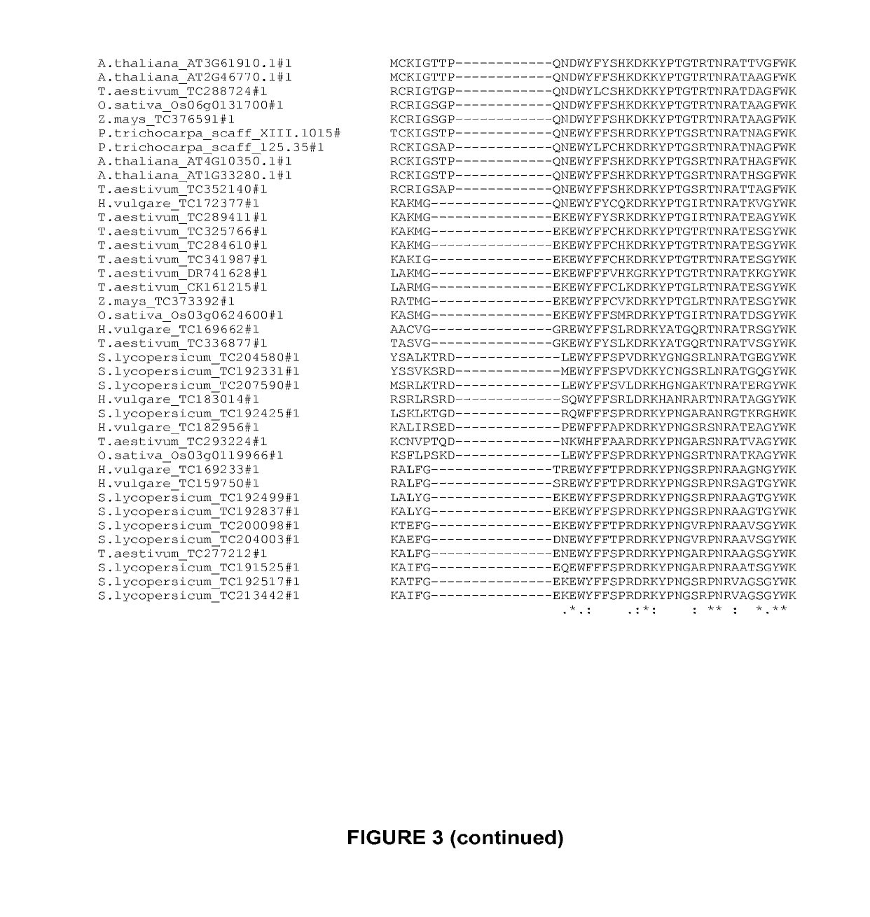

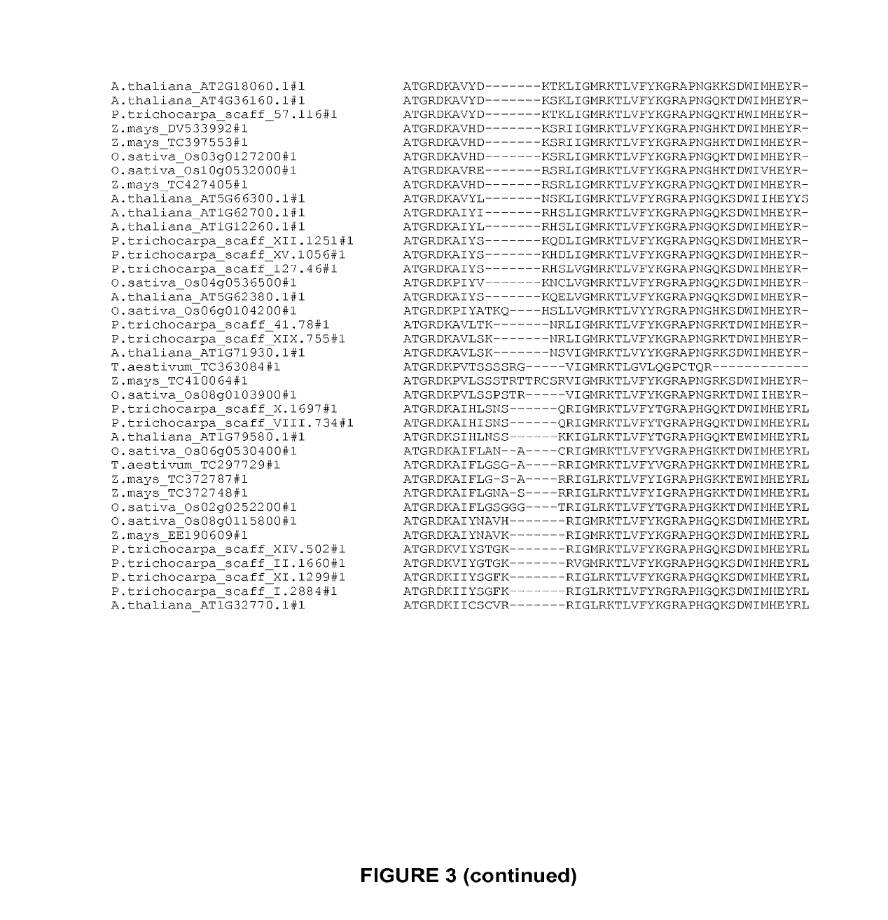

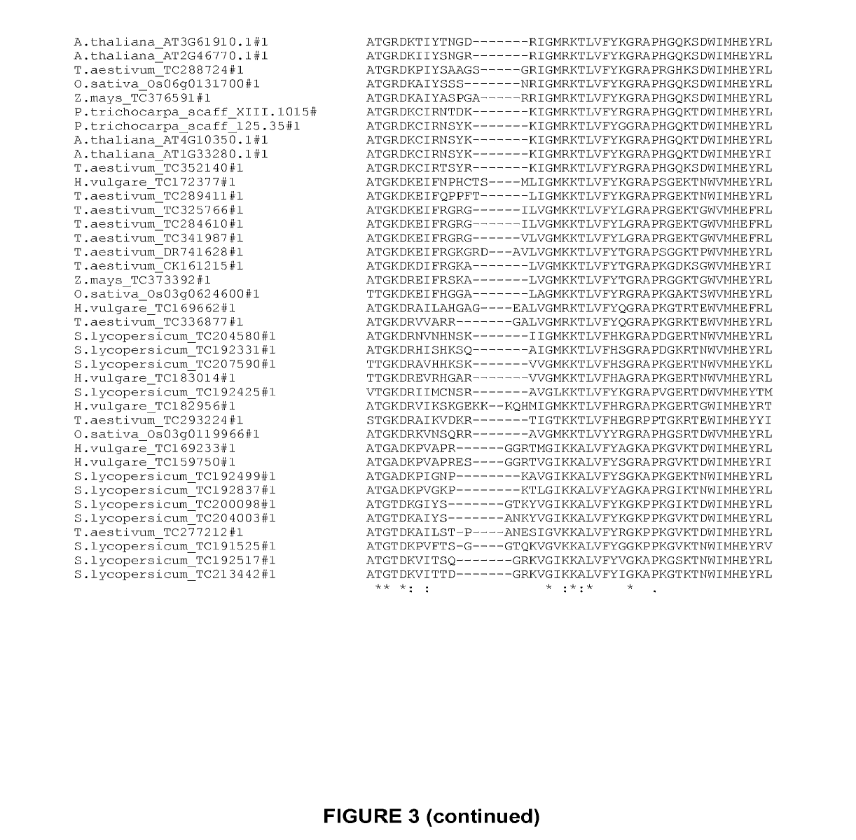

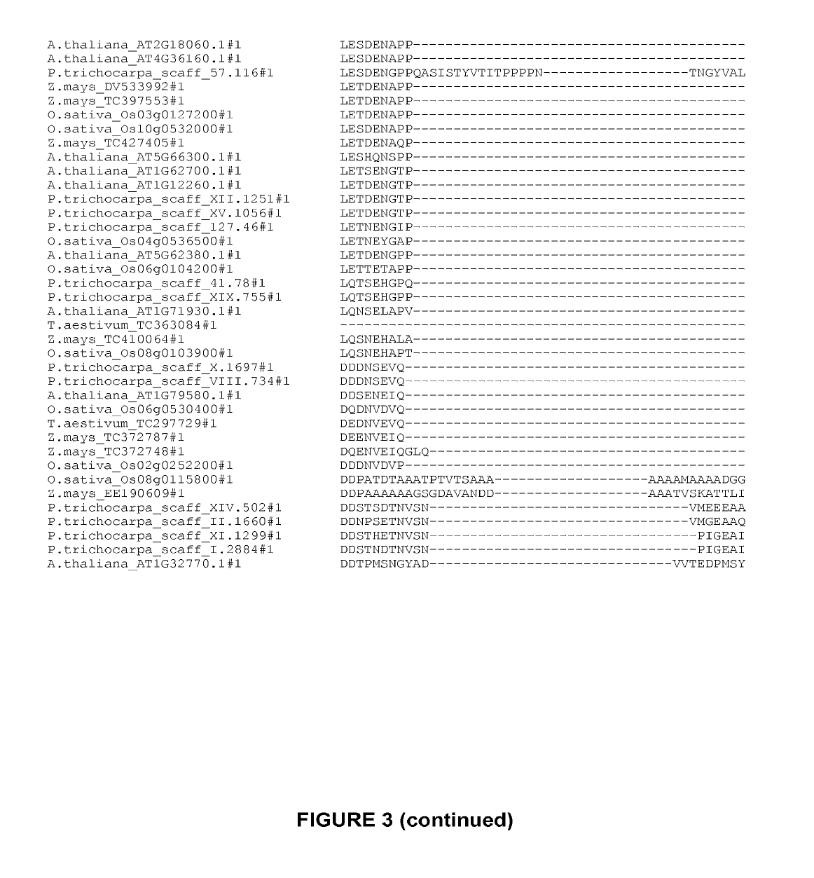

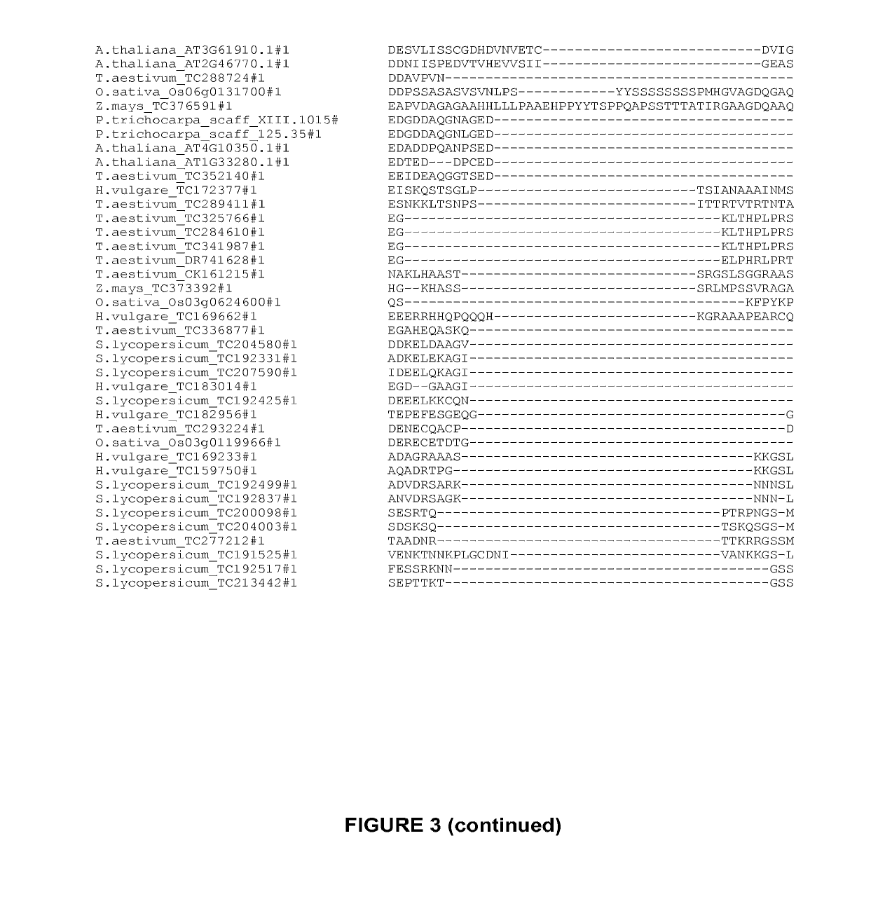

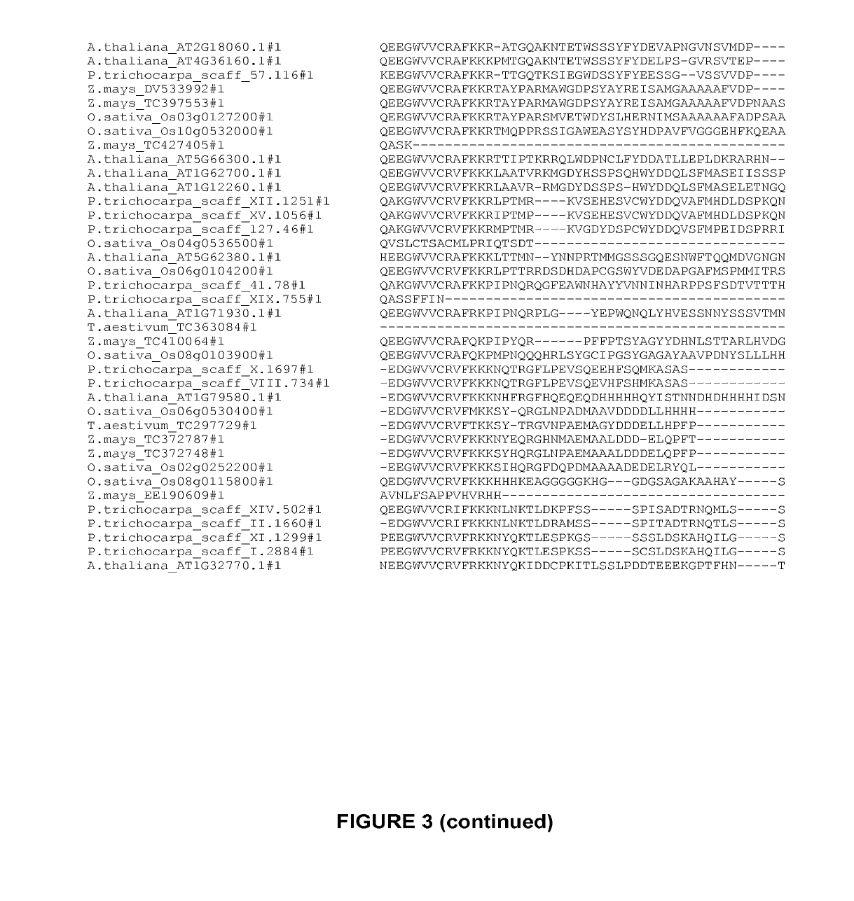

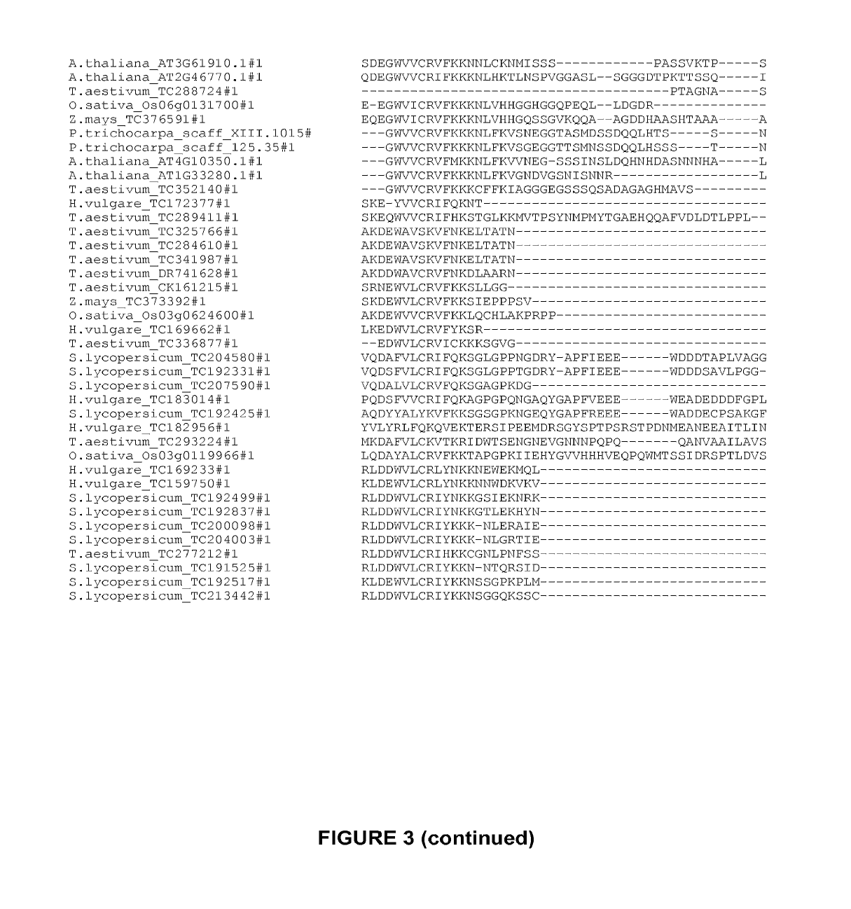

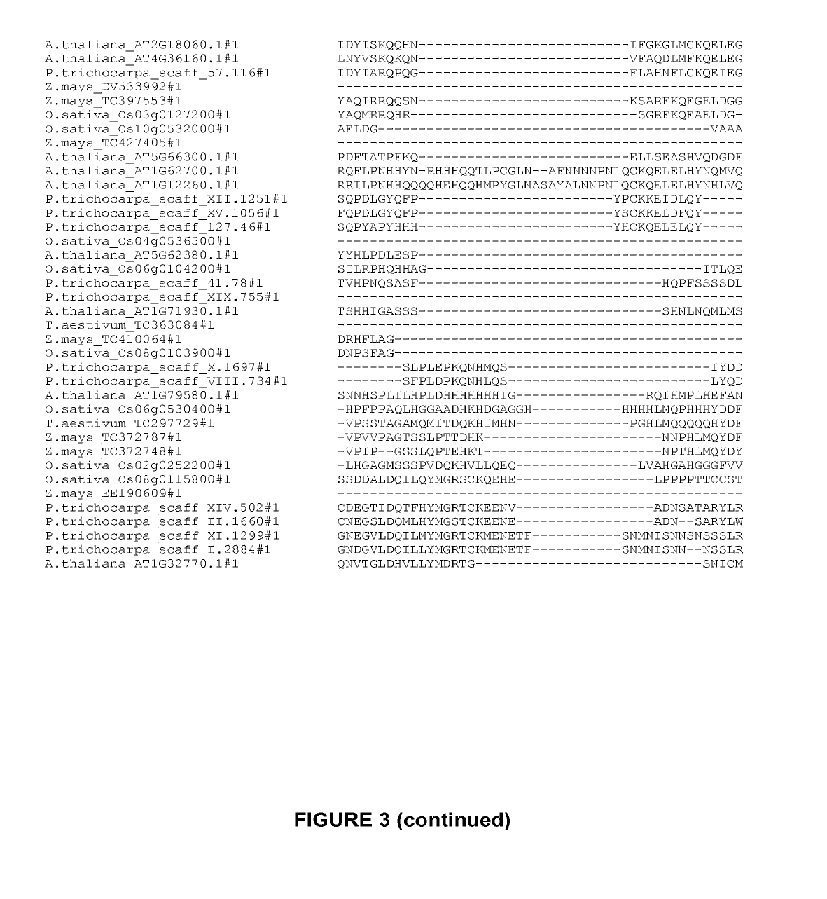

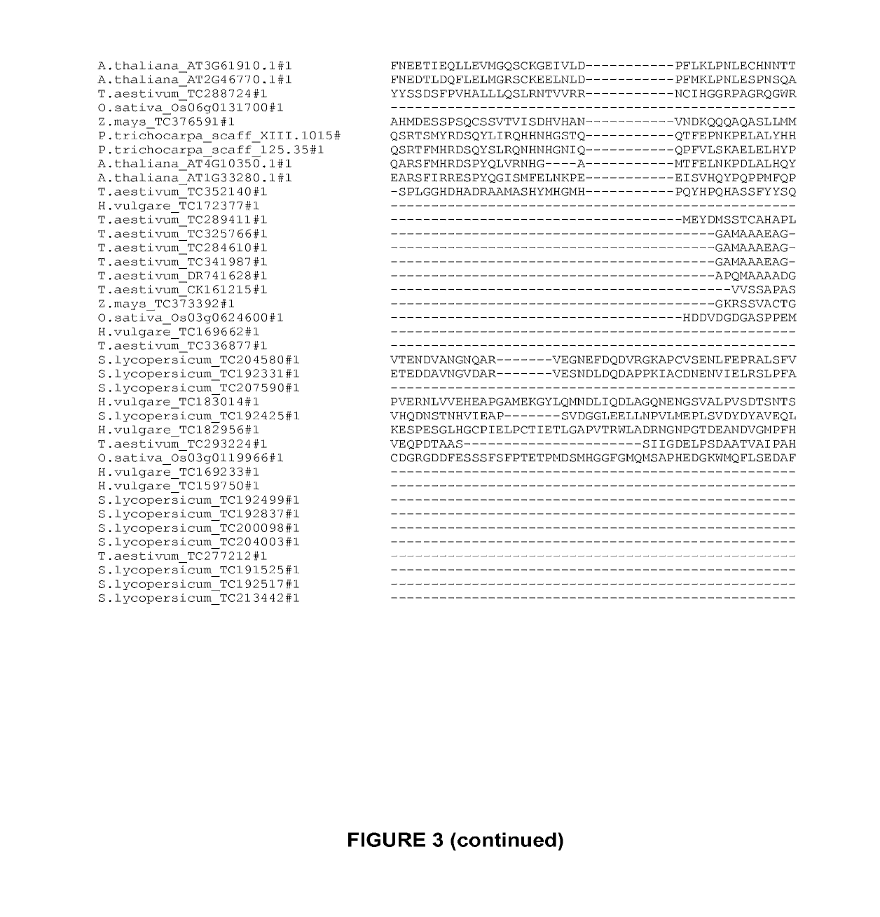

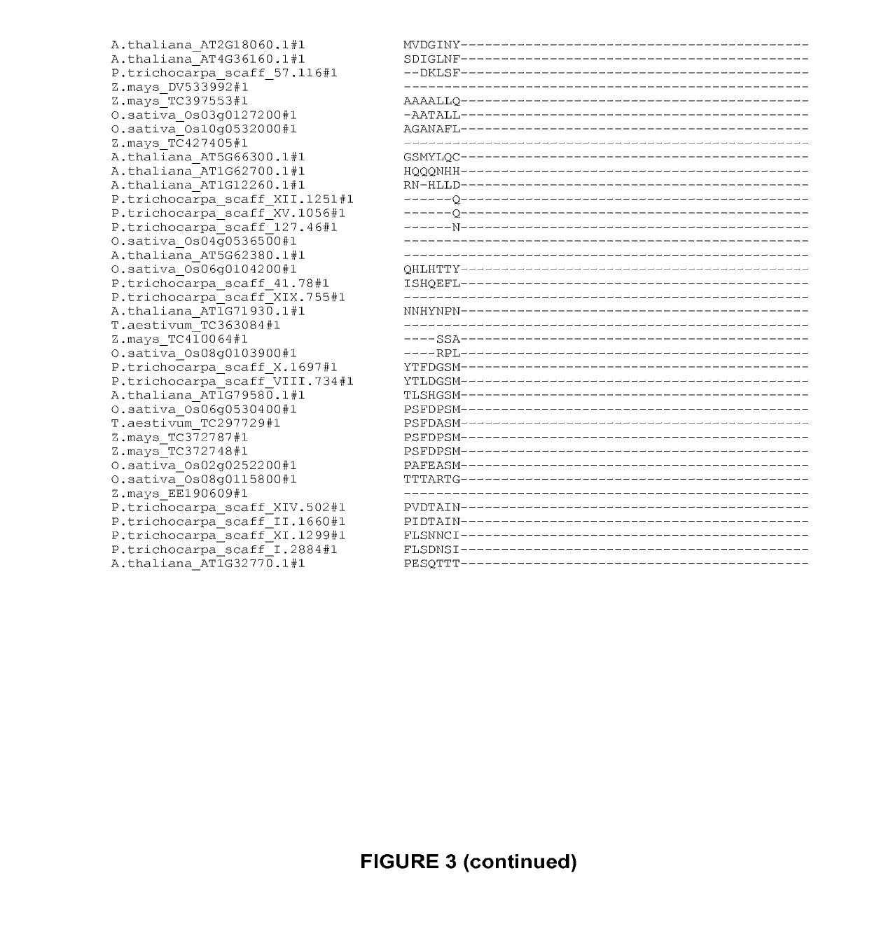

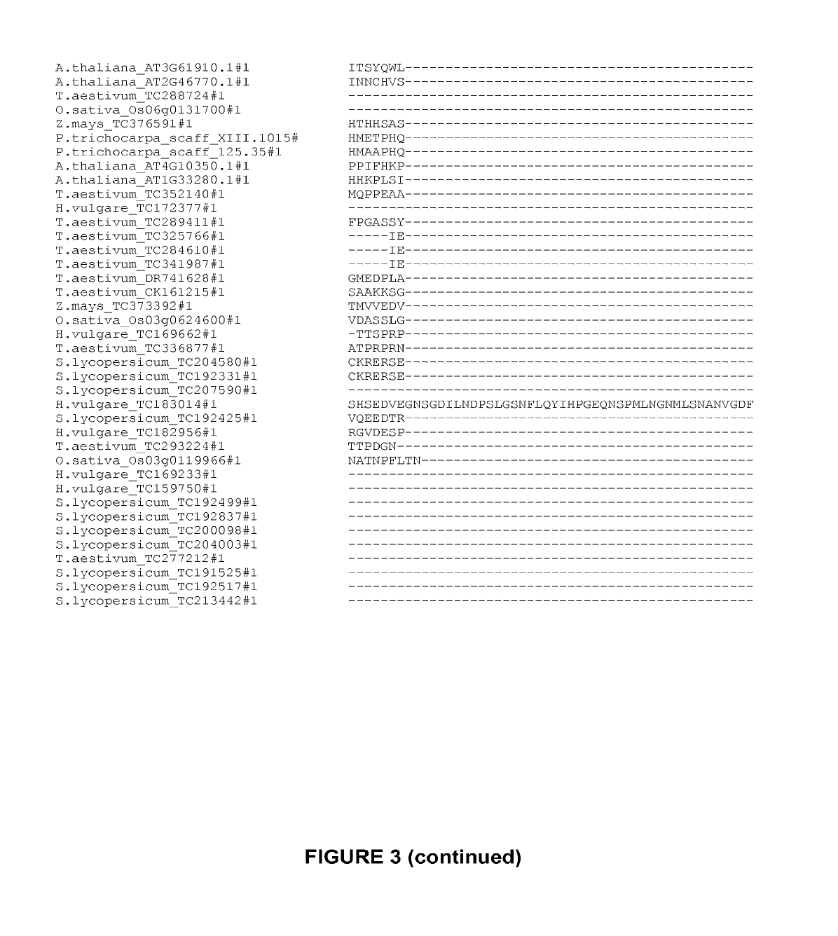

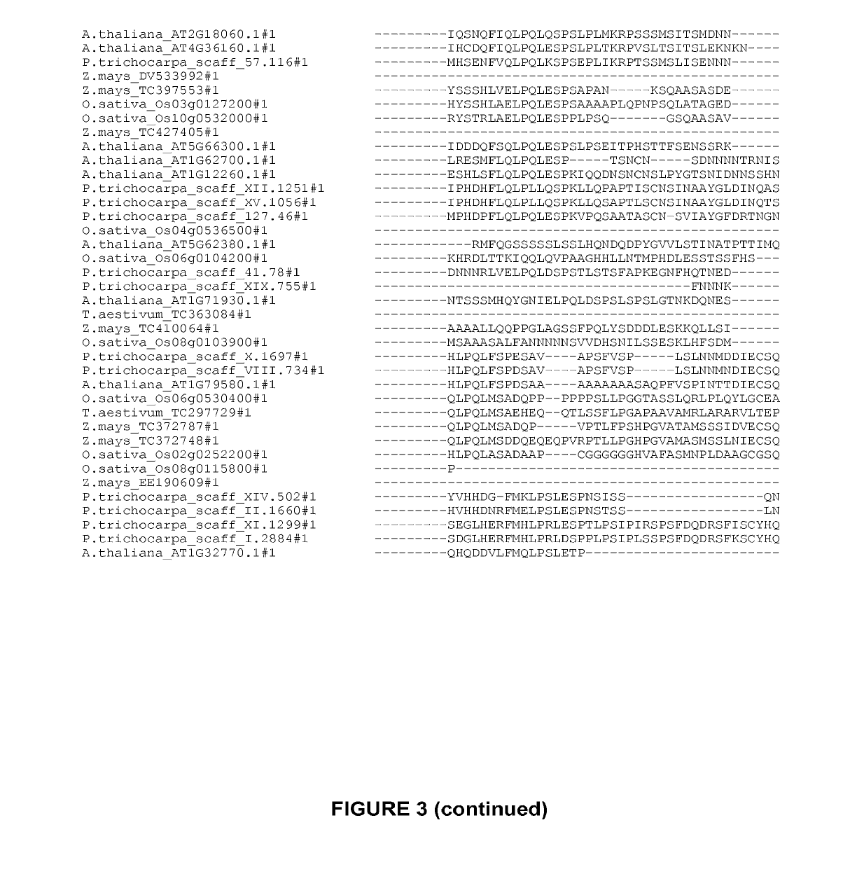

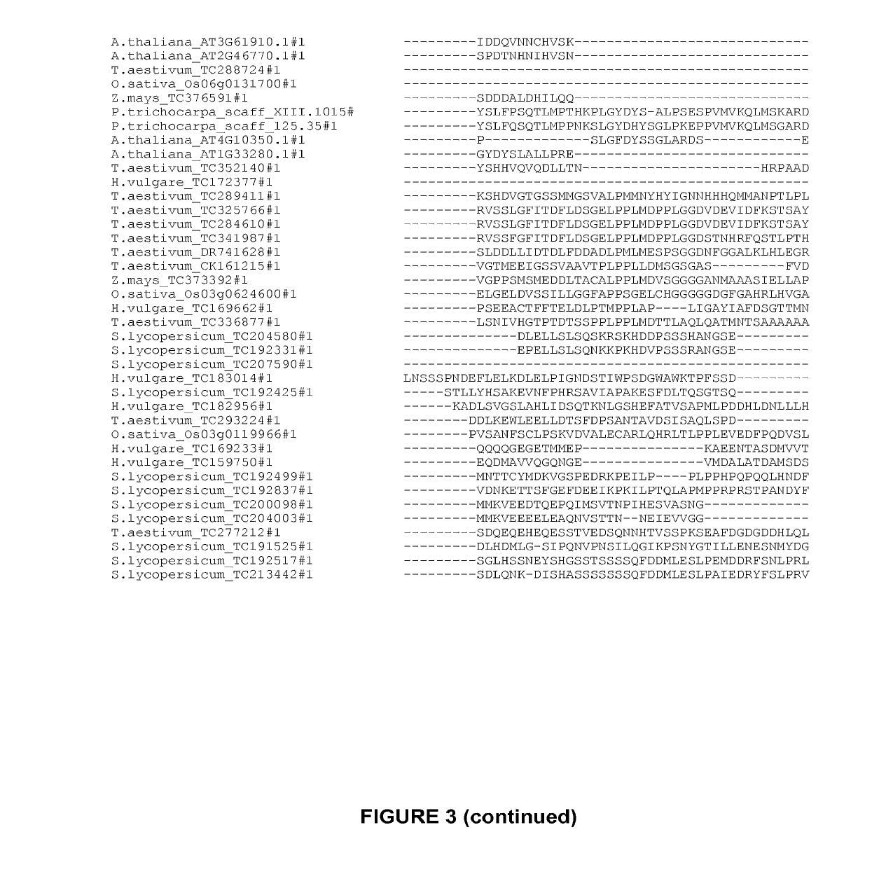

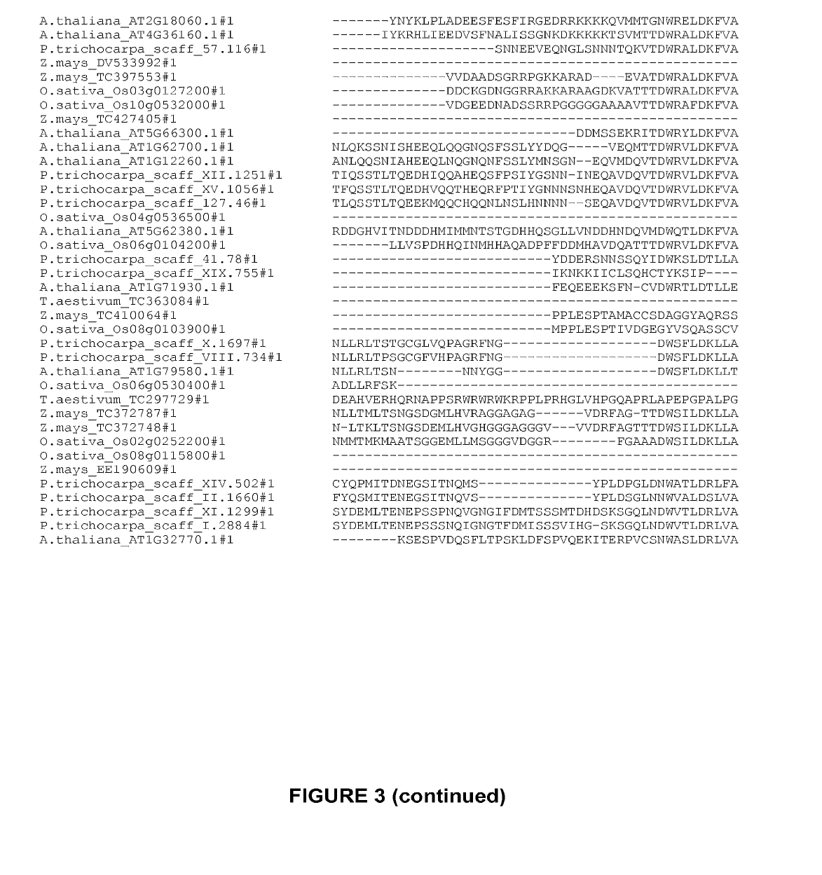

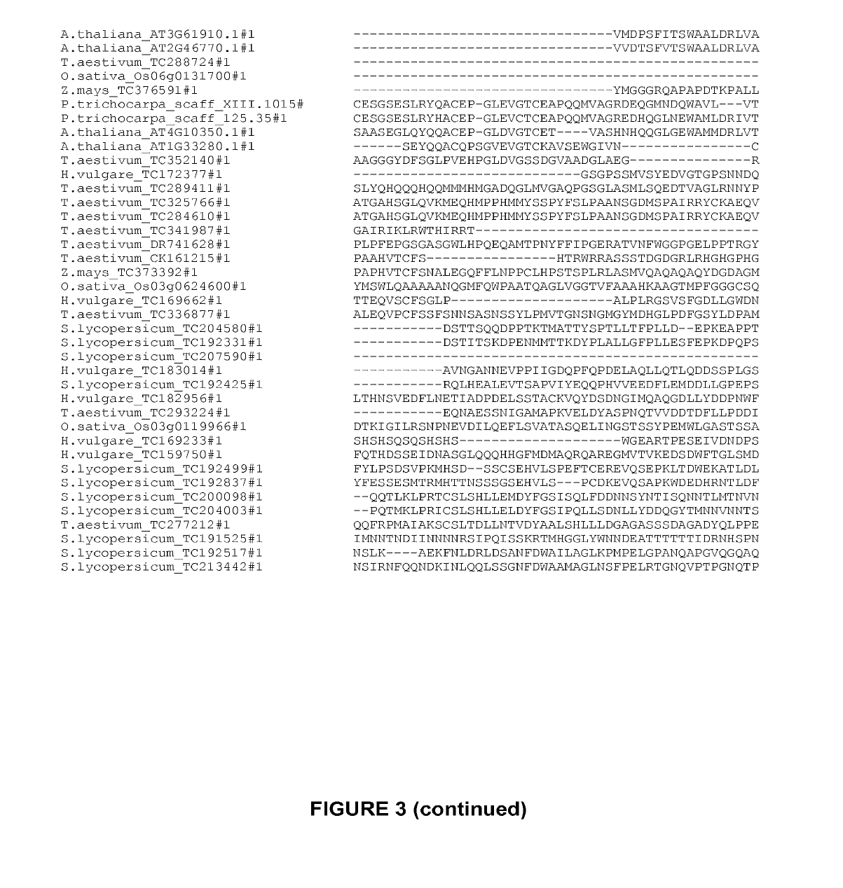

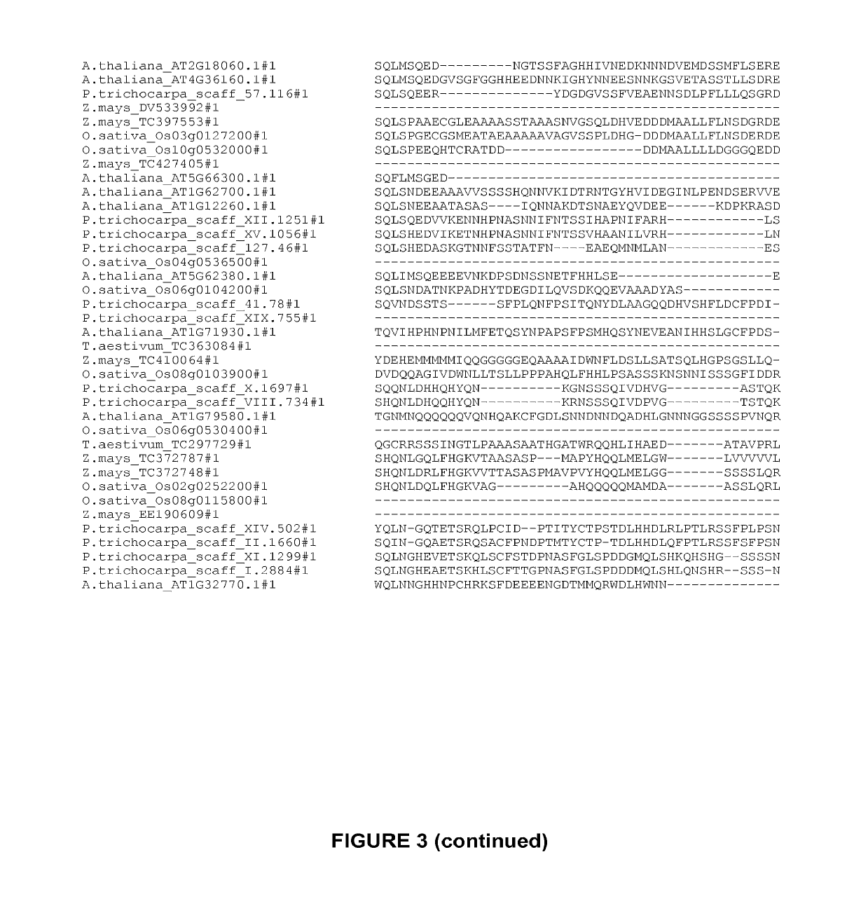

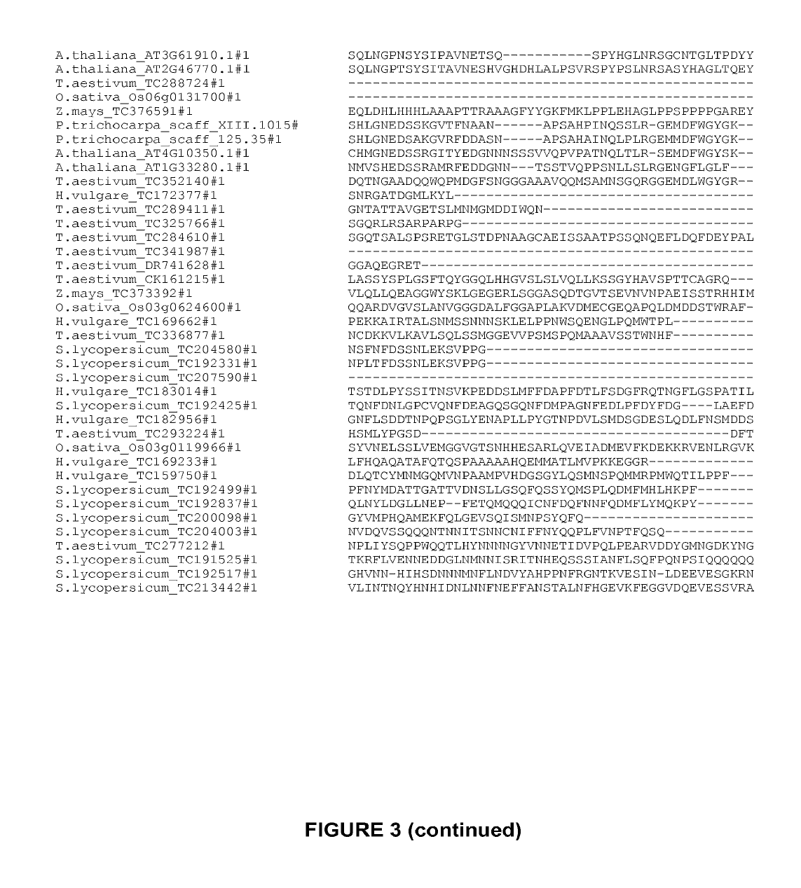

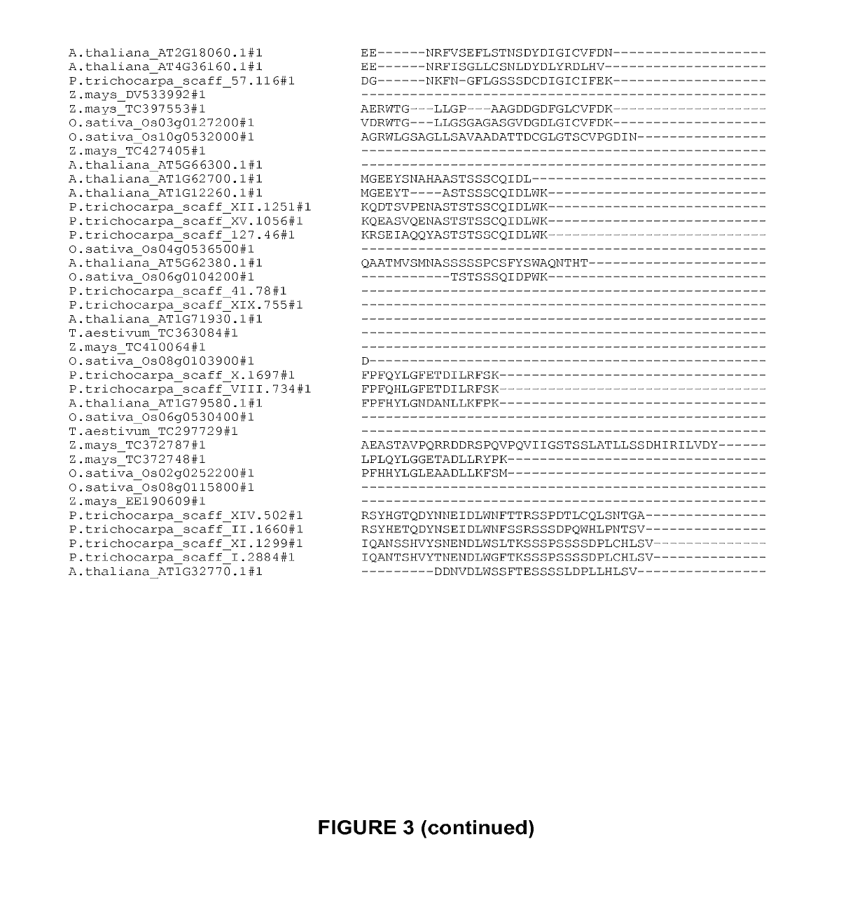

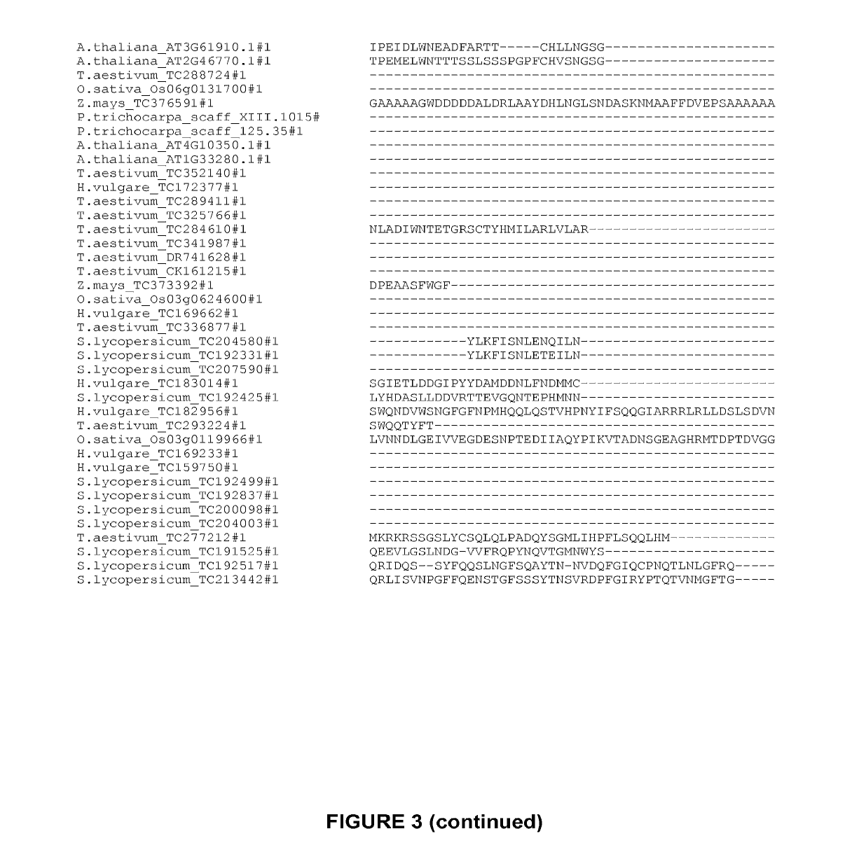

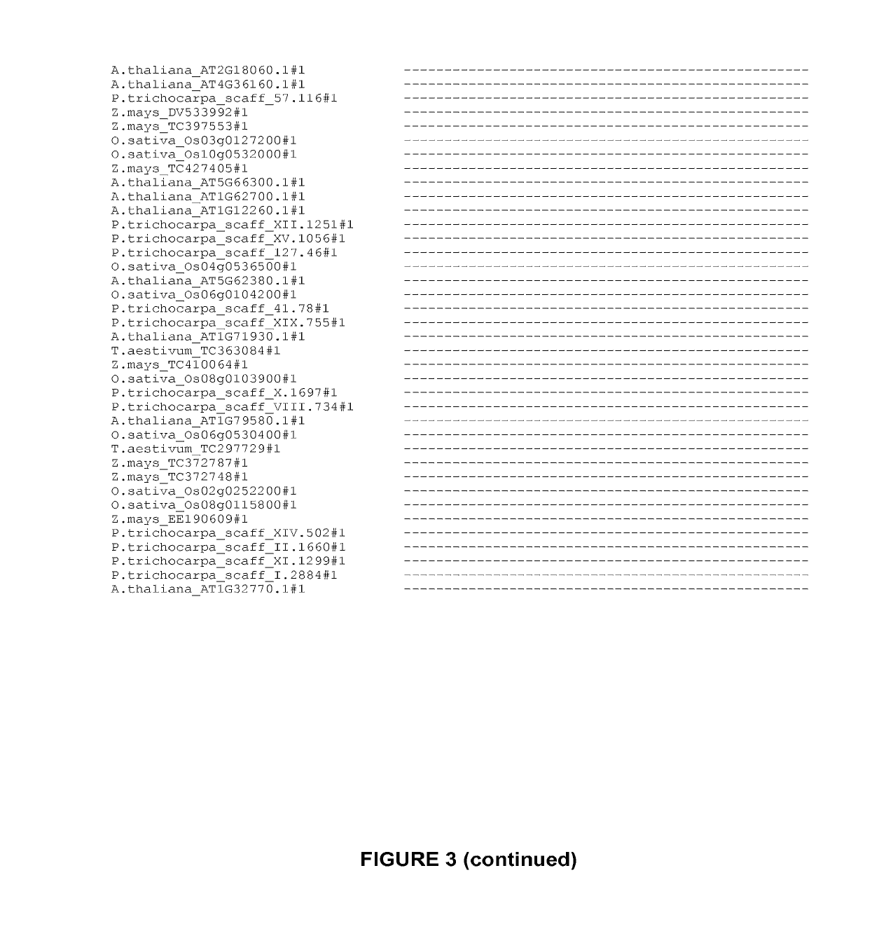

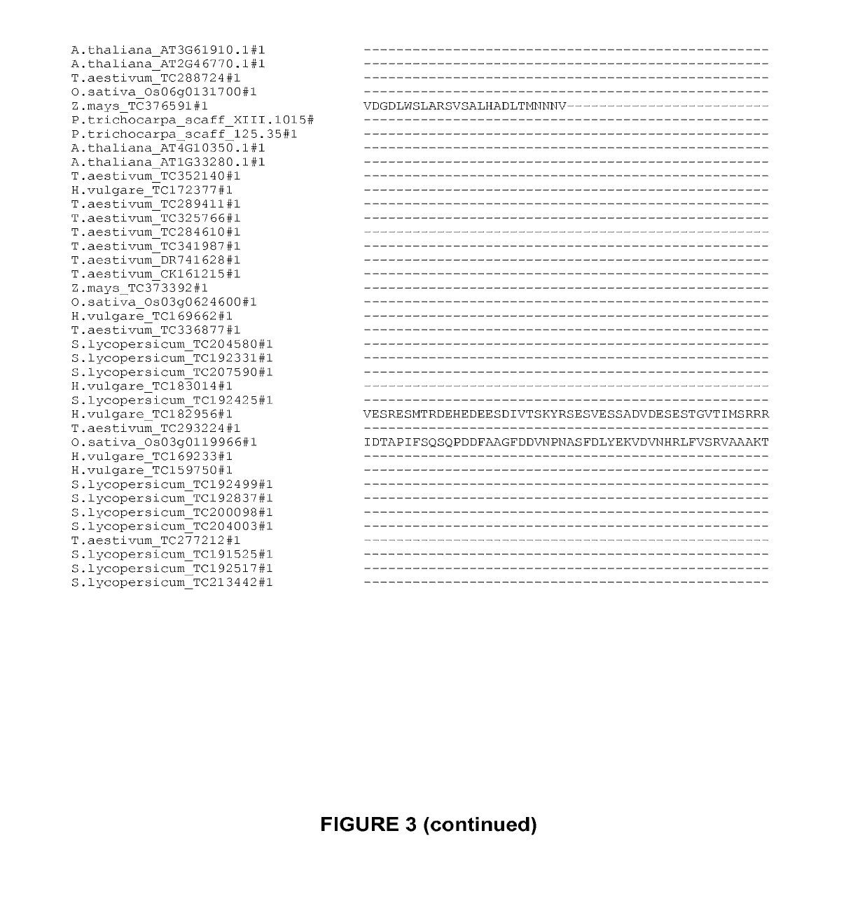

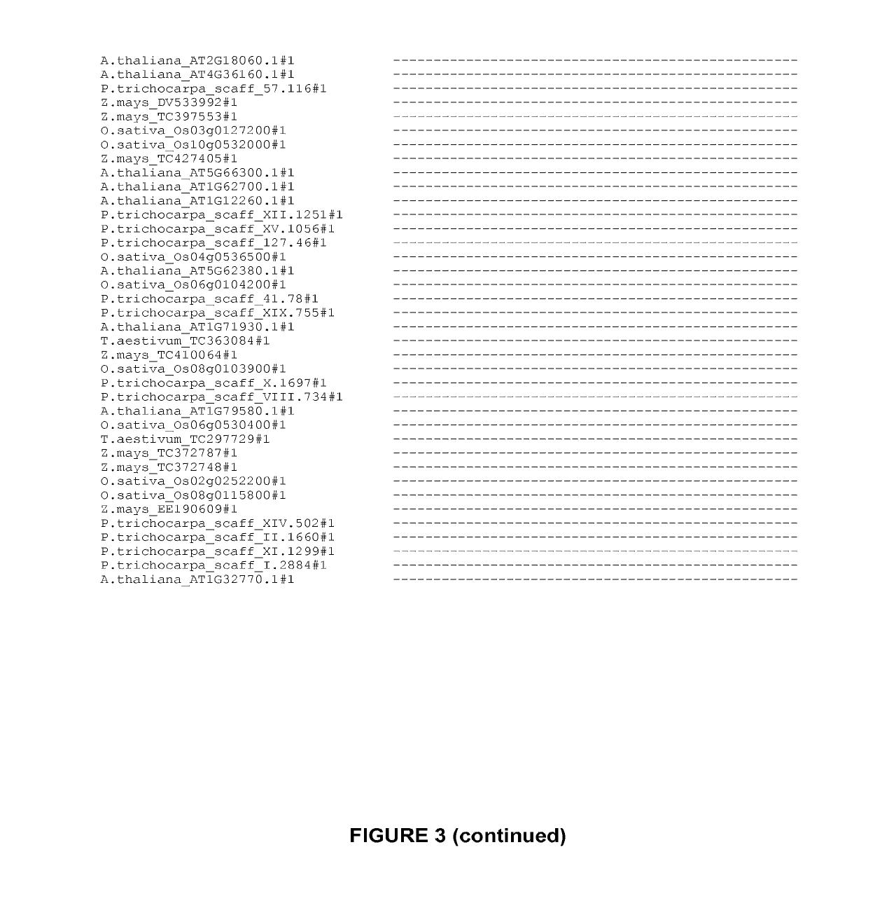

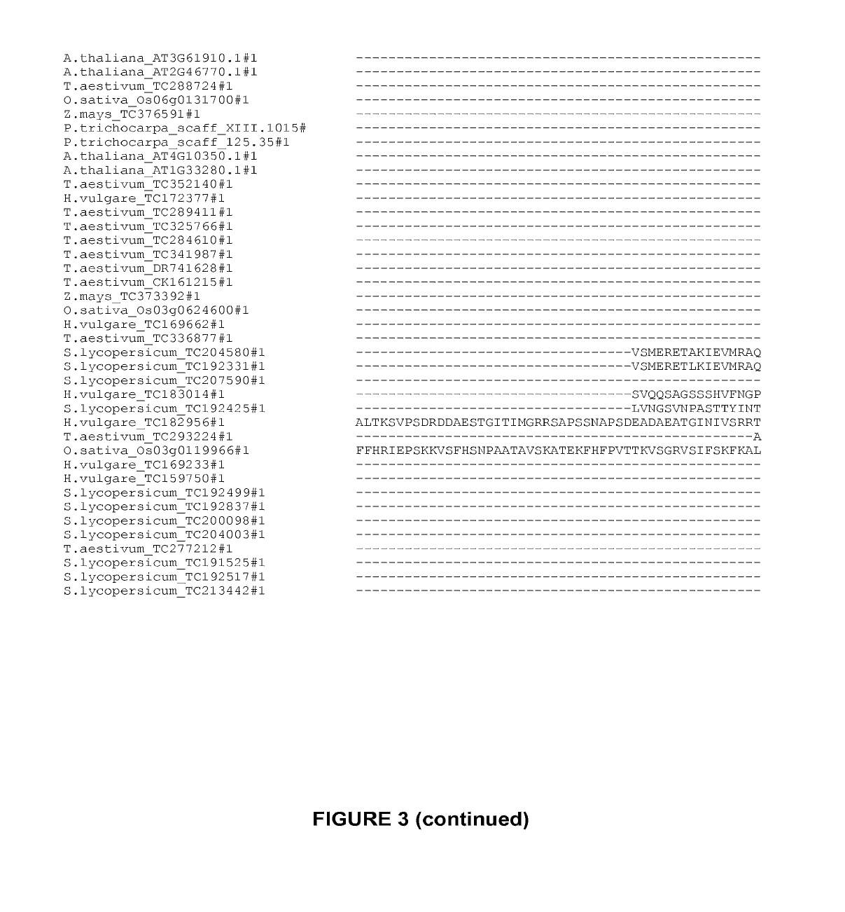

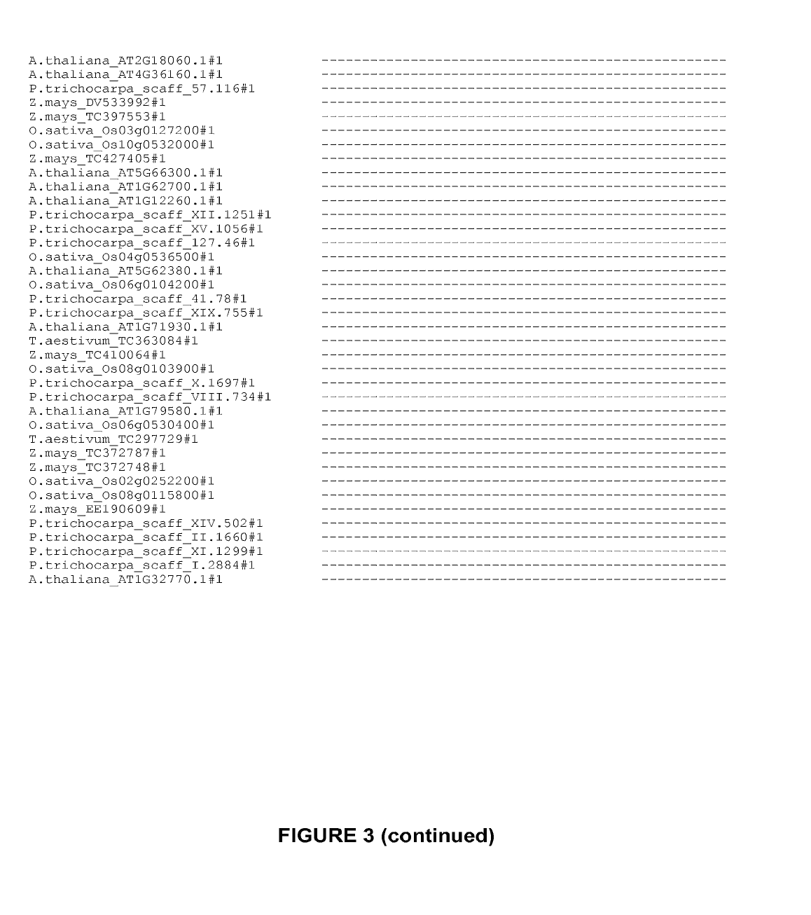

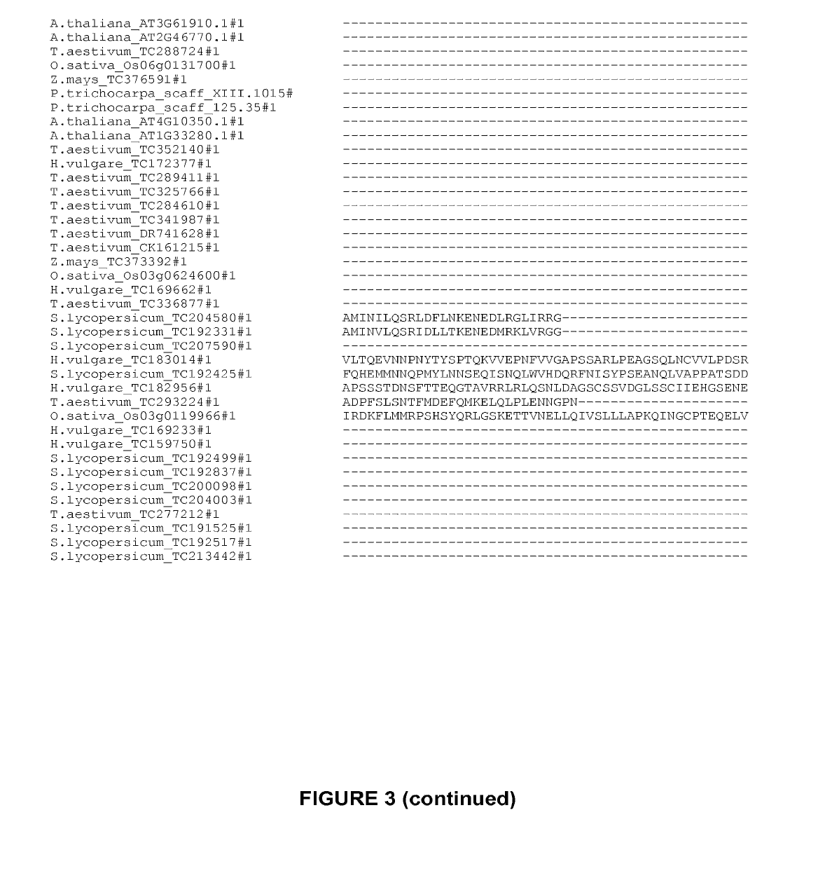

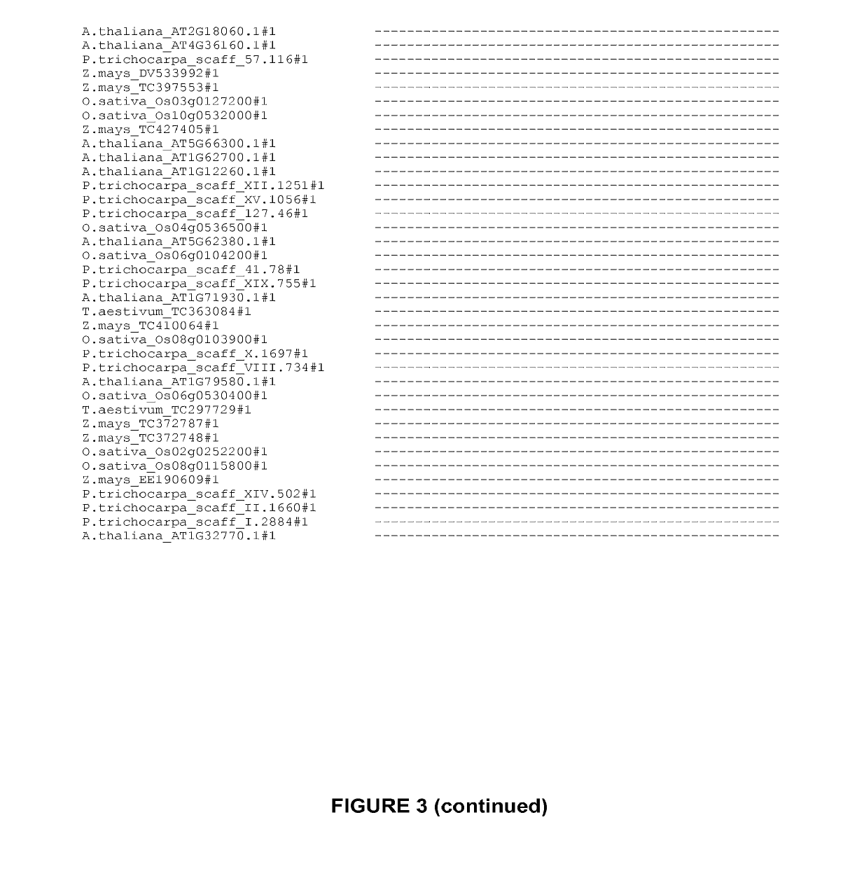

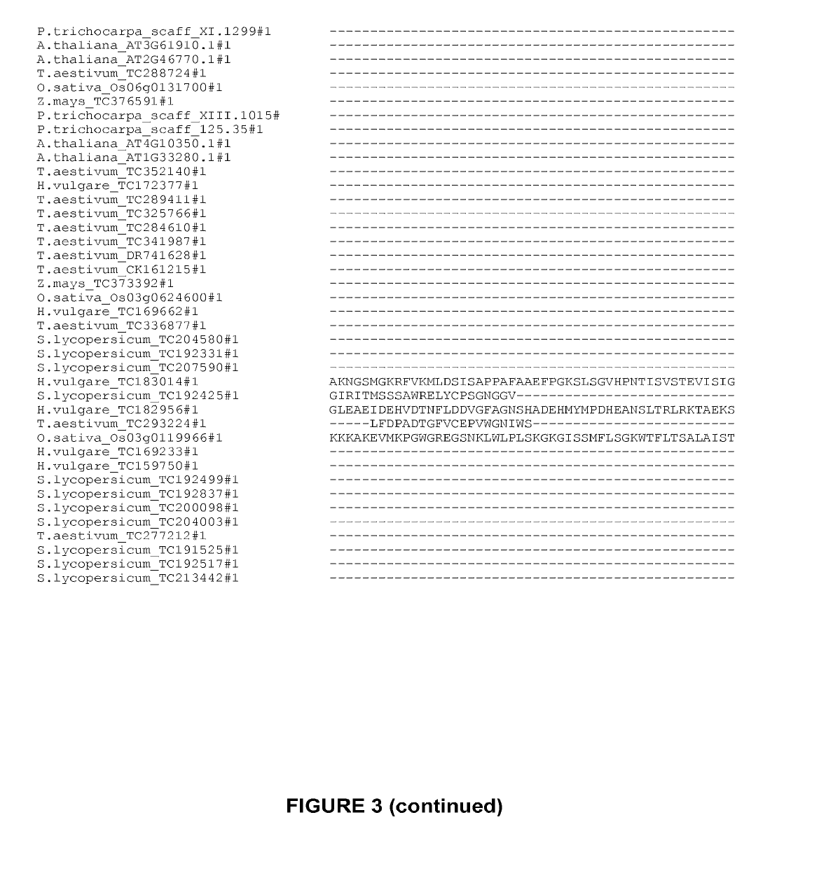

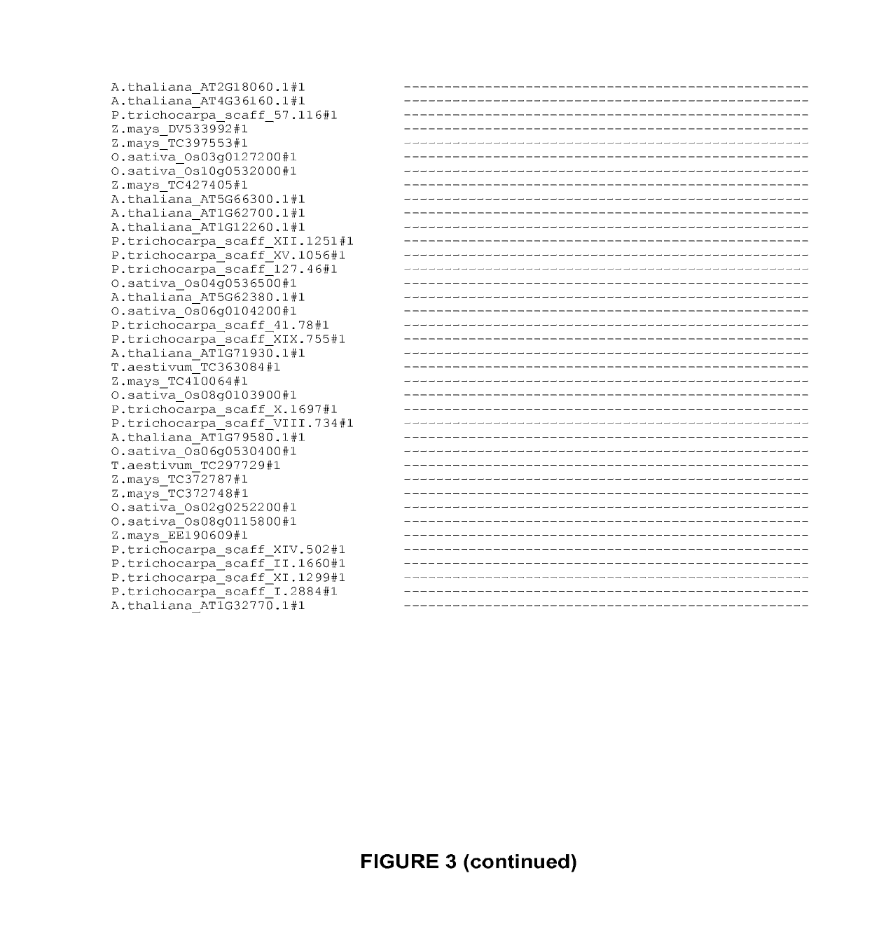

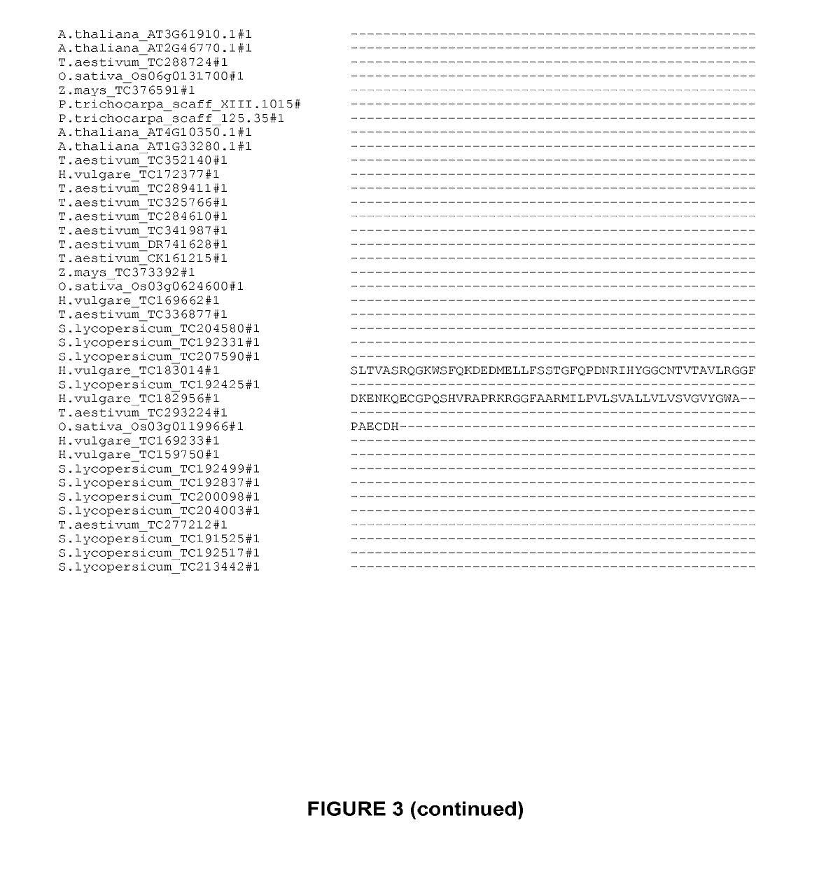

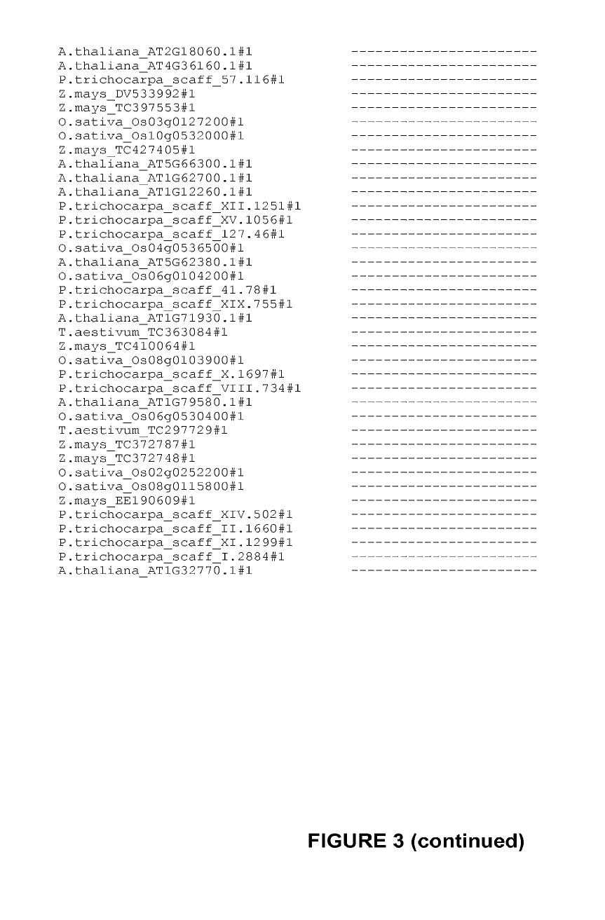

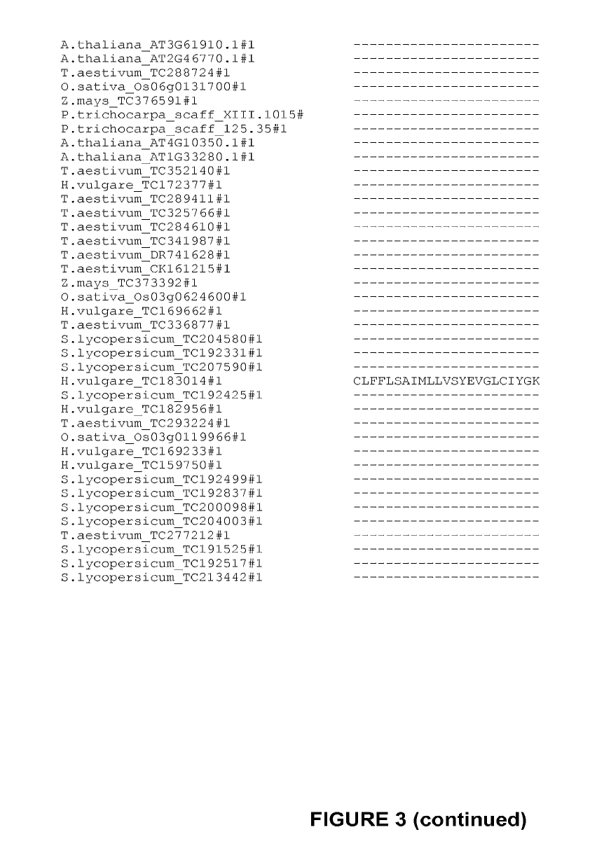

FIGS. 20 and 21 show results for the NAM2 homologue SQ23 (Os03g0127200, see FIG. 3) and SQ9 (Os04g0536500, see FIG. 3) which show comparable phenotypes to plants overexpressing NAM2.

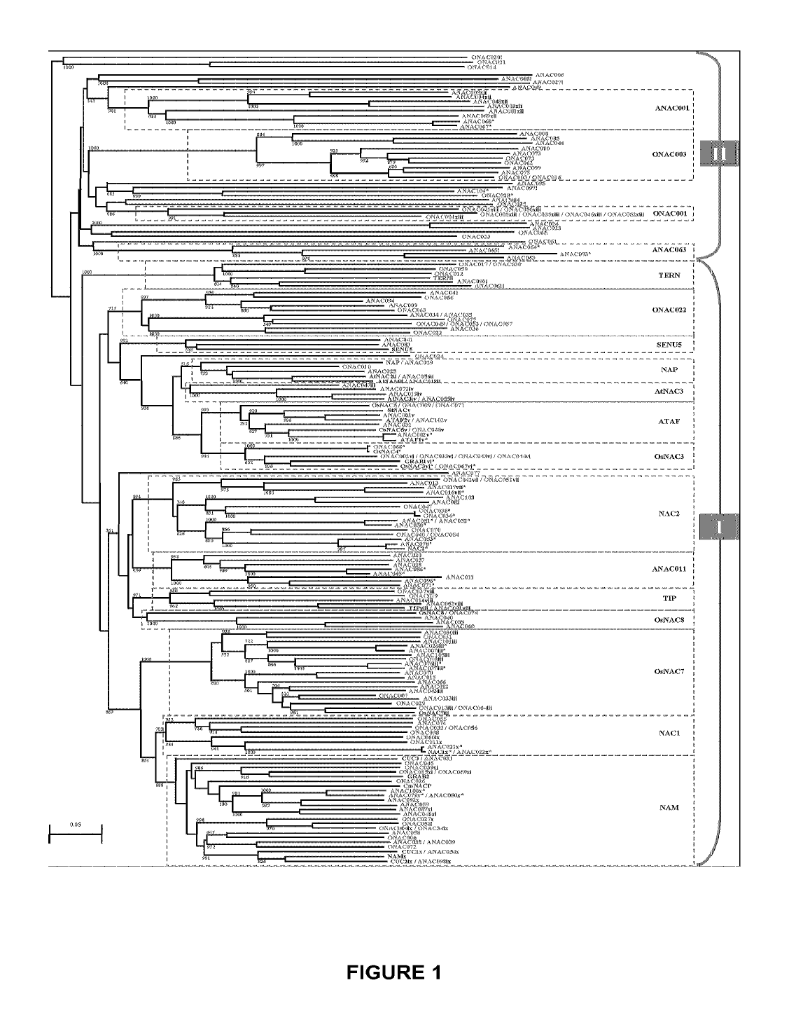

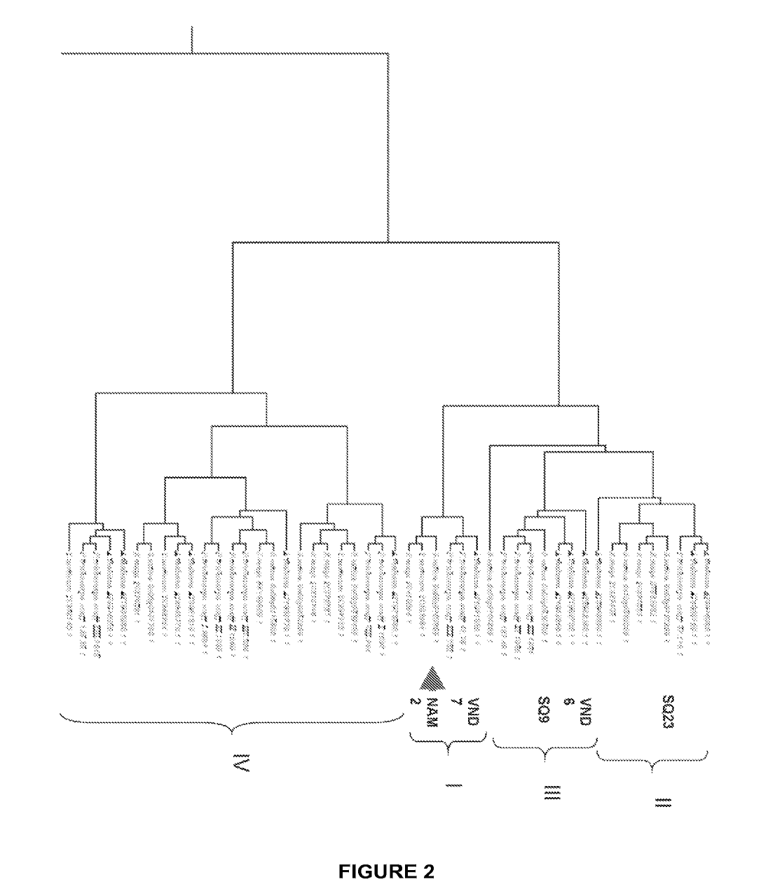

1. A method for modulating carbon partitioning in a plant relative to a corresponding control plant, comprising modulating expression in a plant of a nucleic acid encoding a NAC transcription factor, wherein the amino acid sequence of said NAC transcription factor, when used in the construction of a NAC phylogenetic tree, as the one depicted in FIG. 1, tends to cluster with the group of NACs comprising the amino acid sequence of SEQ ID NO: 2 rather than with any other NAC group.

2. The method of claim 1, wherein the NAC transcription factor comprises any one or more of the following motifs:
Motif I: KIDLDIIQELD (SEQ ID NO: 155), or a motif having at least 50% sequence identity t the sequence of Motif I;
Motif II: CKYGXGHGGDEQTEW (SEQ ID NO: 156), or a motif having at least 50% sequence identity to the sequence of Motif II, where ‘X’ is be any amino acid or a gap; and/or
Motif III: GWVVCRAFQKP (SEQ ID NO: 157), or a motif having at least 50% sequence identity to the sequence of Motif III.

3. The method of claim 1, wherein the nucleic acid encoding a NAC transcription factor is any one of the nucleic acids listed in Table 1, or a portion thereof, or a sequence capable of hybridising with any one of the nucleic acids listed in Table 1.

4. The method of claim 1, wherein the modulated expression is effected by any one or more of T-DNA activation tagging, TILLING, or homologous recombination.

5. The method of claim 1, wherein the modulated expression is effected by introducing and expressing in a plant a nucleic acid encoding a NAC transcription factor, wherein the amino acid sequence of said NAC transcription factor, when used in the construction of a NAC phylogenetic tree, as depicted in FIG. 1, tends to cluster with the group of NACs comprising the amino acid sequence of SEQ ID NO: 2 rather than with any other NAC group.

6. The method of claim 1, wherein the modulated expression is overexpression in a plant of a nucleic acid encoding a NAC transcription factor.

7. The method of claim 1, wherein the modulated expression is the reduction or substantial elimination of expression in a plant of a nucleic acid encoding a NAC transcription factor.

8. The method of claim 1, wherein the modulated carbon partitioning results in plants displaying one or more of the following: increased number of tillers (culms); reduced diameter of tillers; increased fresh weight; smaller plants; increased number of panicles; increased panicle length; increased seed weight per plant; increased seed size; increased Thousand Kernel Weight (TKW); increased grain filling; reduced width of flag leaves; changed vascular bundles in flag leaves; a decrease in the number of large vascular bundles and an increase in the number of small vascular bundles in flag leaves; change in number of veins; thinner leaves; increased number of stomata; changed sugar content, composition, or quality; increased sucrose synthase activity both at heading and ripening, increased sucrose phosphate synthase activity; increased invertase in flag leaves; reduced beta amylase activity; increased starch in leaves at ripening and heading; increased glucose, sucrose, fructose at heading time; increased arabinose and galactose at ripening; increased vasculature and stomatal density; change in auxin flux/sugar flux; increased lignin accumulation; and/or delayed senescence with delayed degradation of sugars.

9. The method of claim 7, wherein the plant having decreased or substantially eliminated expression of a NAC transcription factor display displays one or more of the following: reduced number of tillers (culms), increased diameter of tillers, bigger tillers, reduced number of panicles, reduced panicle length, reduced seed weight per plant, increased seed size, reduced grain filling or fertility (% fertile seeds per panicle), increased width of flag leaves, thinner leaves, altered vascular bundles with an increase in the number of large vascular bundles and a decrease in small vascular bundles, altered number of leaf veins, decreased number of stomata, reduced photosynthesis, decreased fresh weight, reduced sucrose synthase activity at heading and ripening, reduced sucrose phosphate synthase activity at heading and ripening, decreased invertase in flag leaves at heading and ripening, increased Beta amylase activity decreased starch in flag leaves, decreased sucrose, increased glucose, increase in fructose at ripening, decreased arabinose, decreased vasculature and stomatal density, a change in auxin flux/sugar flux, less lignin accumulation, and/or accelerated senescence with faster degradation of sugars and less sugar transferred to the sink.

10. The method of claim 5, wherein the nucleic acid is operably linked to a constitutive promoter or a ubiquitin promoter.

11. The method of claim 1, wherein the nucleic acid encoding a NAC transcription factor is of plant origin, from a monocotyledonous plant, from the family Poaceae, from the genus Oryza, or from Oryza sativa.

12. A plant cell, plant, or part thereof, including seeds, obtained by the method of claim 1, wherein said plant cell, plant, or part thereof comprises a nucleic acid encoding a NAC transcription factor.

13. Construct A construct comprising:
(a) a nucleic acid encoding a NAC transcription factor, wherein the amino acid sequence of said NAC transcription factor, when used in the construction of a NAC phylogenetic tree, as depicted in FIG. 1, tends to cluster with the group of NACs comprising the amino acid sequence of SEQ ID NO: 2 rather than with any other NAC group;
(b) one or more control sequences capable of driving expression of the nucleic acid sequence of (a); and optionally
(c) a transcription termination sequence.

14. The construct of claim 13, wherein the one or more control sequences comprises a constitutive promoter or a ubiquitin promoter.

15. A method for making a plant having modulated carbon partitioning relative to a corresponding control plant, comprising introducing and expressing in a plant the construct of claim 13.

16. A plant, plant part, or plant cell comprising the construct of claim 13.

17. A method for the production of a transgenic plant having modulated carbon partitioning relative to a corresponding control plant, comprising:
(i) introducing and expressing in a plant, plant part, or plant cell a nucleic acid encoding a NAC transcription factor, wherein the amino acid sequence of said NAC transcription factor, when used in the construction of a NAC phylogenetic tree, as depicted in FIG. 1, tends to cluster with the group of NACs comprising the amino acid sequence of SEQ ID NO: 2 rather than with any other NAC group; and
(ii) cultivating the plant, plant part, or plant cell under conditions promoting plant growth and development.

18. A transgenic plant having modulated carbon partitioning relative to a corresponding control plant, said modulated carbon partitioning resulting from increased expression of a nucleic acid encoding a NAC transcription factor, wherein the amino acid sequence of said NAC transcription factor, when used in the construction of a NAC phylogenetic tree, as depicted in FIG. 1, tends to cluster with the group of NACs comprising the amino acid sequence of SEQ ID NO: 2 rather than with any other NAC group.

19. The transgenic plant of claim 18, wherein said plant is a crop plant a monocot, a cereal, rice, maize, wheat, barley, millet, rye, sorghum, or oats.

20. Harvestable parts of the plant of claim 19, wherein said harvestable parts are seeds.

21. Products derived from the plant of claim 19 and/or from harvestable parts of said plant.

22. (canceled)
